SEQUENCE LISTING (208 sequences)

SEQ ID NO: 1 - DNA, 1858 bp, synthetic construct
  source 1..1858: mol_type = other DNA
agaatgtaccttattttgagcaatgtttgtacatatttaaaagtaatttctattgttatttctgtgaactgtctacacctaagatgtctaaattctgattatttctctaatgggatattttgtacctgtcttggggatttcccagaggtttttcaacatggtctcacagtccagataataccacctgacttaaagaggtccatcccacaatcaattgtcttttggggttatctcaccgtgcagcctattttaagaaaaacatacctttgtcctgccaatggtggagtccagcttcttctctggacttcttttctgtctatgctcaatattcaaagaagttatccagtttcttgcctttatacacgagttaggcagtttctacacctgcaaaatccagaggacgtaggccaagctctctggtaggttctttttaaccaccctaacttggtataaggtaaagaggagggaatacttctccccttccaatggaagcaaaagagaaatgtcacagcacattgacacatctctagtgaaattctcttcccagccatttgttttagtcttctggtttgttcttataaatccttacagtgagtattgaatctaatgctgcaaaatgggattctccatctcttagcatgcatgtcctgtatagaaagtctcaaactccaaagcagcaaaaacaaaacaatcacattcaatgtactattagcagcaactagactgatgggttttttttttaaattaagctacttccttaaatatttaaccagttttatgcatttgtaaagaatttaacaaatggagatgttagagacaatttctatggcatttctatatatcttggactaccttttcaaggatatatgcacagcaaagagcattagaggctagtctgtccctttgggtcagacagcagaattatttattgtccagtataataattacagagaacatctccatctctgaagacattccataataataaagtcttcaacctctcaccagaagaaaacttgtttccatgccaaggtaaagataatgcctatctttgaagaggaggatgaggaagttgccatcagcttctgtatgaatcagaatgtcctaagctcagtgttccttggctgtgaaactgtatgctcagcatacacctggatctactctgcattacccatggacttgggggacaaggagaatcaatgaaaatatggacctcatgttgcctattgtgccctgaacaataaactgtgtaaattcatttggattcctggtttactcatttgccaatgttatgaaagtgtggaaagtctgcctagaagttacactgatgttcaggaattgcttggcaagttgataggaaagaaagggacctgtggcatatacagctaggttctccattttctttccttctttcgcatggaaatggaaactcaatattagttttccccaatccttcatagaataaaaactgcctttcccagcctttcttgcagctgagtgtaactacaggtctaatatttggccaaagaaatgcaaggagaagtagtctgtgcaatacctaagtagtgtccatagaggaggcacacccttctccttttattttctccttcttgcaggatggaatgcagatatgctgactggagcaggagcagccatattgcattataaggtaaatttaggaatggagacatgtttttcagagcaaaggaggaaaggttcaagacagaagaagcatttgtggttattgatatcataaagcatcacttcagcagccttagactgctgacttagaatttgatgagaaaaattaacttctactttctttaaactcatttttttgacgtttctgtcactcaca

SEQ ID NO: 2 - DNA, 1612 bp, synthetic construct
  source 1..1612: mol_type = other DNA
gggggtctgatatgagtttgtgcatgtgtgcagggtgagtgagacaatccatatatttgagaaaagacagtgaaatctttaaaggtaaaaataataaaaccagctgtggcacctattggataaggaattactaactgatgatttagaaattcctttatgtgcgttaggagctcatttgttatccccattttacagatgcgcagccccaggttcagcaatttgctctaaatgagactgtagaggctaattccagcccccggcagctctcaccttaagctgcctggctgaggtcagcagtaagttacctggcagcgtccgggggcctgcagccgaggacagggctggaactacacttcccagtgtgccaggcggggcggggcgcggcggggagggcgcaggctccttcccagaagcaaaagcggaaacaaaagagtctcgccggcgtccccgcccgcacactcgcgcacactcgcgctcgggcgcacacggagcagggaccggcgcccggagcgagccagggagcggctaaccggggacccaccgcgcggagccagcctagctgccagcgagcccaacccgcgacgacccacgcccctgagccccgcagccgacccctgccggccggtgtccccaccgccatccctgacccatggcgctgaagaggattcagaaagtgagtgccggggccgggcctggggctgcggggcagcggccccctgcccacgctgactcggccagtggtcccgagagacatgcggggagagcaagggatggaagggtgggccggggtgcgggcagggagctgaaggctcgggccgggccagcgggcatcggacaggtgaggtgggagcagggtcaggtcccaggcggccggaggagcttgaaggctgggggcccgcgcgcgttcgcgccggagccgctcccggagccccagggtagagagcctcttgcgcgtccagaccgtcccgaaatctgtgcttcacggtcacggccgggttttgccctccatggggctagttttgacccctgtcatgctgtgttttctccaggctgtttcttaagcctgtcctcttttatgttcttgcttacccggcctccccgggagggcaaggaaggtggggtgggcctgcagcctttctccgcggagctgtcgggttggttctttctctcctctgcggttggctcttgctcagttgcccgaatgggctggaaagcccatttgcggccaagaagtcaggagaaggctcctgccccttctattttggtgcctctgctgctgccacttcaggtcacatagctgtagcgaaaacatagttgctgatacacaacaagatgacctcctggctgtgattcacggtgagtcaagccaaacaggtttttttccccctcatgataaacttagatgtaaagttcgagacacaacataaactgtcgcaaataaattgttttgatgctttgacacatgggtatagaatgaggaagaaatatatatcttcatccaacaactgtttacccatctcaaccctgtgtatggaatcgtctttttttcacagtatttattgccaccatttcagatgcgaacatccttcagaattcagattcgggcatttct

SEQ ID NO: 3 - DNA, 554 bp, synthetic construct
  source 1..554: mol_type = other DNA
ttagctgtgttttctattcctctgcttgagagctctctttgtctttttttgagttttactttatttttaatttacaaataataattgtatatattcatgaggtacaatgtgatggtttggatacttggatacatgtttacatcacggaatgatcaaatcaggccaattagcttaaccatcacctcaaatatttatttctttgtggtgaaaacgtttaaaatcttctcttttagtgttttgaaatatgcaatacattattattaactgtcacacagtattgtgcaatagatcaccagaacttattcctcatgtctaactgaactgtgtacccattgactaacatctcccctttctccagccccctccccagcctctggtaaccaccattctactctctgcttctattaatttgactttttttttttcagacagggtctcactctgtcacccaggctggagtgtagtggcatgatctcggctcactgcagcctccatctccttcgctcaaatgatcctcccacctcagcctcccaagtagctgggactataggcat

SEQ ID NO: 4 - DNA, 828 bp, synthetic construct
  source 1..828: mol_type = other DNA
aagagttgagaccctgcctcaaaaaaaaaacaaaaaaacacaaaactatcacattttgtattatcatatgcaaattgatctgcctacagccaaagcttaccattaatctacagctatgccccatcattcaaacttgttcaaaagattgtattatagaaattttacttctagatagaatgtggattaaatctcattgtagattttgtataaaatgatgaatttctgataccacatacctttgttcaggctttgtttgtctcttgtttttgttgtgttgtttttaacagcttcatcactggtccagctgtaataccagggtacttctccgttgatgtgaataatgtggtactcattttaaatggaagagaaaaagcaaagatcttttatgccacccagtggttactttatgcacaaaatttagtgcaaattcaaaaactccagcatcttgctgttgttttgctcggaaatgaacattgtgataatgagtggataaacccattcctcaaaagaaatggaggcttcgtggagctgcttttcataatatatgacagcccctggattaatgacgtggatgtttttcagtggcctttaggagtagcaacgtaagtacaaaatatgattaaacatttttaggccctaacacctgaatttctgaaccaggaagacttattcaaaattgtaataaatttagttcttctgggattattgaggccaggtacagtctttctcagagataaaaacagggtgtggtcgcatccttcaataacaattatgatgtttttaagagtattttattctttaatgaggagatgagttactttttttctt

SEQ ID NO: 5 - DNA, 1120 bp, synthetic construct
  source 1..1120: mol_type = other DNA
catttttttcttcttttgcataatagaatggtatagtaaacacaacactggctttgttgtttagacaaacaagtcttcaattccaattctaattctgattgagaaaacctaacctctcagaggcccagtttctctgttaaaaggggatcataccacttacttcataaagagtgatcatgagcattcaatccacttatttgttaagggcctaccgtatgccagtcagtgttccaggcttatgagatacagcagagcacagaaataacaagtccttgcgtgcatggagcttacattgtgctggggagatagcaagcatataaatacgtggaaatgtgtaatgtcaggaagtgagaagtgctaatataaaaaataaagcacagcgtggggacagagagtatgcaggatacttagaggggatggatgttttggcctgtctgaaagggtggcatttgagcagtgactgggatgcagtgaggagcaagccatgcaaacatctggggagacagcatccaggcagtgggaatggcaggtgcaaaggtcctgaggtaggaggatttctgtgtgcttaaggaggtaggttggtgggcctggcaacatgggcagtggcctgattactagggctttataggtgatggtaagggtttgggttaaatgaagaaacagggattgtgagtctaacatctcctcgcttctccttcccccacttccaaacaaatattggtatttttaacataatggccaacaagtttgtgctctattgatttttaaaattagataagctagttcttatctacggactcactttgtctatgggccaaatgtcctgtggttatttgtaaagtataatctggagatattgattctgtttttgcaattacccattagaagaaattgtgttaatctttccctttccttctcctcccttctaaattctgttaggtgtgtgcttcggtacttaataaacaagcctgattacttacatggaaagaactagaataagctttttttttttttttttttttgagatcgagtctcactctgttgcctcctgagtagctgggactatgaaactgcaagcacccaccaccacgctaatttttgtatttttagtagagatggg

SEQ ID NO: 6 - DNA, 1599 bp, synthetic construct
  source 1..1599: mol_type = other DNA
ctctgcctcccaggttcaagcgattctcctgcctcagcctcctgagtagctgggattacaggcatgcgccaccacacccggctgattttgtatttttagtagagacagggtttctccatgttggccaagctggcctcgaactcctgacctcaggtgatccacccgcctcggcctcccaaagtgctgggattataagcgtgagccatggcacctggccaactctttcaactaattgccaatcagaaaaactttaaaatctgcctgtgacctggatacactcctcacacgcacttcgagttgtcctgcccttccatatggaaccaacgtaagtcctatatgtatttgactgatgtctcatgtctccctaaaatgtataaaactaggctgtgtctgagcaccttgggcacatgttctcaggatctcctgagggctgtgtcacaggccattgatcactcatatttggctcagaataaatctcctcaaatacttttttgtcaacagtccactgggctcaggtgagcagggcctgaaacagatgacatggaaatttataataaaataaggggcccgtgccttaagacataccacaaagtaaatataacactgtacaggctctctcgggtgaccctggactacagaatgtgggttgtggggagggagagacgtggactttttcctgcaggtcatggcttgggtgcacctagaaacaggccaataccacctcttcttttctggaaaggtggggcctgacagctttgtgtggggaaccagtgtcctgttacagttcaggtctaaaatgcctgctgagtaaataaacatgttgttctcaacctaaaccgaaaagcctgatatttgtaggaataaaattgatctttcctaccctgagcaaagtgctccaaatatgctcagtcatggagtggagacagataagtaaacagacgctgggaatagagtggagacagataaataaacagatgctggggatatgtcgtgatacagagccaggccagagtcaagcaaggtgctgcattggcattctgccggcctttcctgcaggaaaaggtgtgaaaaagctcttggggtgatgggaaggggtgtgctgttttgaaatcggaagataaaaacagagccatggaggttctgcgccatcgccttggaacagatggtatctgcctccacatctgcaatgactcagatctctgctctgggcatacctgcagtcaaatcagggctgcccagacatcctgtgtttgtgaaatgctgagaaaaacagaaggggactaactggcttctagcagttgcctactcatccaccctcttccaaacacccaggctggggagtaacattttggggatgagtcctcgtaatgctgcttctctctagagagcaatgtgtgaaaattgacccaaggagttgtctcatggggtgaggcatgattggctgtcttttctcagagcctccctgaggctcatctacccccatgccctgagctcactggggcctgcctggccaaacagaatatacccattaagggaaataaatttccaggccacaataacaagcatcaccactttgaaaagaaagaaccataaactgaa

SEQ ID NO: 7 - DNA, 1462 bp, synthetic construct
  source 1..1462: mol_type = other DNA
cagatgggcagggtaggggttgggtgggaacagaagttggagagtggatgtggctgccaaccacacaagttgtgccaacccacagattgaggaaagatgctaaatttggaatctggcaaaccagtgtttggttcttagctctgccacttctaagctgtgtgaaacttggttgaggtccctaacttctcctgagggtgaacaactcacaaagttgttttgcttattaaatgtgataacacctgtaaacatctaacagagtgcctagcacatagcagggatctagcaattgaattagggttatttgtttctgtctactgattgggtattgtttctgacacttacccaagtgtgaatagcctataacactggtataatttgtgaaatgatgctgccatctagtgaaaaccaagacacacacacacacacacacacacacacatacacacacacgtgcgcgcgcatggacacccagcttcaccaatgacaatatggattggcatgttttagcctcacaacacagagccctggggctaactggcacctagagaggtcatctcggccagtgccttccaaactaccagtgctgaaaagccagttcaaaaaattttgaacccattgcacaccaatatttttgtgaaataataaaaataaattactggaaaaatgaaataaaaaatatgtataaaatacaaaccaaaattttagaactgttagattcaacagcaaaaaattgctatatacatctctgaccaattgctttcagtttctgtgcttatctctctacgacctttgtaacacacagtgaaccagcgctggcccatggatacactctagtagccccaatctagctaaggcagccccttatagttaatcaatcctgtcaaacaggaaaggctggcaaaaccactggtctgcatgtactttgtcctttacacaaggaaggatgcaaacgtggaaaactgagtggacatggtgttcaggagattgaggctcagctaaattccagcttatttacctgcagttgcttacaaagtgtttggacataattgtgtaaagctagggttttttttctggtttttaaaacaggtaaaggatgtcacagcaccacttaataacatttcttctgaagtccagattttaacatctgatgccaataaattaactgctgagaacatcactagtgctacgcgagtggttggacagatattcaacacttccagaaatgcttcacctgaggtaaaactcacagagctttaaaaaaaattttttttttatttttagagggggtgttgggaaagaaagagagatgtcctgaataccactccttattccacaatatggtttgagcatggtttaagttgcttaaaggcattttgcatagcactcctgagagacgctcaaagattaaaaaaagggcctcttgtccaaatattttattttaggatattcctaatgatcatta

SEQ ID NO: 8 - DNA, 1507 bp, synthetic construct
  source 1..1507: mol_type = other DNA
gctagaaaagtccaagaatccaggaacggccccacatcaggcaaggattagagaagggacagcggctccgtagctggatctcctagactcagaggagtttccaaacaacaggcgtctttttcagtattataacagaattaacggtggcatccaacattttcctatgcactggttttaagcttatttctataaaacctcatttaattctcacaagaacatcactgggaacgcactaaccctttacaggcacgaggaaactgagacaaggagagattaagtaacttgcccaatattttactgcgaggagcaggcaggactggctggtaggtgcgtgaggacggctagggcagcagaccctcaacctcgaaggacgacgcgagcccgcctcccacgcaagaggttctgccgaggggacgcaaactcccgcagcctcccaaccccgccctccagcgctggccgcggcttctcttccccaagccaaggacgggttcattgctggccccacgcctaggcaaagagggctcggagaagtcttggagacccggagctgactcacctccctgggagatatctcagatccgacctcgccttcctggagcggaagtgcctccgagtgcccacggttcgtggactccacttcccagaattcctaagcggaagcgtctcagagttctctcactcctggactacagttcccagaatgctccaagggcagcgcttcccaccagctgggctttgttttgactcaagccggttgggtttgagccacttctctggtgctactactgtgagattcgtttttgttttattcatttttaaaagctcatgagaaaataattgtaaaatttttaaagtgatgacatataaaaatgaaaagtgcagcttgtattattaaaataagttagaaaccaaaatttttcgcagaagcagagggtaggctaaaatgggaagcgtaaaagtgacttaggctgagctccgatggacgctgaacgttaaggaacaccagatctcaaacttgtgtcaaatgccggctccccaaatatcatcaactcaacctgttgataagacaaaaaacagaatttattgttgattgcagtaatggagaacaacacttcgacggagcgttggcaccttcacagagcaaagtgtcaaggcaaggttggaactgattgagaactggatgtttaaggcgggtctttcaatgcaagggattttattagtattgcataatgatcatgatactacagtccaggattggtggatatagcaaggatttttgaagcaaagaatcttaaattgcaatctattgacactttctttggaagagattgaaaatttgatgggtctctgtggaagttcccataatgaacaattaaacagtttgcttgggcaggaatctcctggaataaaggcctgctgatgaagacagctgaacaatcatgttagtgccgaaagtaaattgtgatgaaacgggaaaagttcacttctccctctcccagggcatgtg

SEQ ID NO: 9 - DNA, 1660 bp, synthetic construct
  source 1..1660: mol_type = other DNA
acgatctcggctccctgaaacctacacctcctggattcaaccgattctccagcctcagcctccagagtaactgggattacaggcgcgcaccaccacgcccagctaatttctgaatttttaaatagaaacagggtttcaccatgttgcccaggctggtctcgaactcctgacctcaggtgatccgcccgcttcggcctcccaaagtgctgggattacaggcgtgagccaccgtgccggtcctccaagacagttttaaaaaggaaaatactcatgaccagcttggggaaaggtggcaaaaacctcgtttctacaaaaacaaaaataaaaataaaaataaaaatttaagtaaaatatctcaagaactttttatattgtgttgaaatagtaatgttttggacatattgggttaaattgtatttaaaaatcaatttcatctgtttcattttacctttagtgtggttactagaaaatttataattacatttgcggctcacatgagactagcattttatttccattggacagcgctggctgagaacaaaacctaaccctctgtgccgccctcgcggccgggatgcggtgcgccccgggcctccccattcggaaaacgaggagcctgggcgcaacggggccggcagccgccttagctacccggctggcgctggggaccccgtcccccgaactgcacaccgcgtggcgggggaaggggtcccagccctcgcggagcacctttggcccagggcgggagctccgagcgccggcttcccgcgccccgcacggcccggcccagcctggaagaaggcctgaggaaggcccgaccgcgccgcccgcgcccttaggcccggcccgaatgctggaaagcccgccggcccgccctcggctcggtgagcctcggctcacctcagctgccggtcgtacttctgctccttgagcagctttcccagctgcgccatggccgcgcctgccgcgcggaaaacagccgagcccctgcggagcgccgccaccagctccacaagcgcgcaggcgcactgagcgccccttcgccgctaccgtctcctgcgcgctgcctgcagggggcgccgcactgcactgcgcacgcgtcgccggtacgccggggcctaagcggccctggtacccgagcgggtctggggctcttggtccccatcggccagaaaggcagacccgctagcctgccaatttgccctctgtccatgcagtaattgggggcgactggggcattggaatcaggcatacttgtatttcgaccacttaactggctgtgagatctactctttacctctcaaatcctcagtgtgctcacctgtgcagtggggataaatgggtcccaagtgtcttttgaccttaaatgagattttgaatacaggaggccagactccccccaaatcttgcatgaataataactttcattttgatgatcattatttttcacccatttcttcctctcatgaaaagcccacaagcctattttgaatcacaaagctaggggctccctgctgcagatgcagacaagtccagggctctgttcttggagagtttagtctcatgaaggaagagggacagcaaacaagcacttaccagacaatgtggccttcctctagatagagggaagcagggtgctcaggggtgcacagaagaacct

SEQ ID NO: 10 - DNA, 753 bp, synthetic construct
  source 1..753: mol_type = other DNA
gtgattagccagagctgataacctgttattaggggattttctctgtgaataaccttgacgctggagtgaggaagggcttagcagcattgaaataaacgcttcccattggctggagaaatttcctcgtgagccactcctcctttaatttatcccaccctttctcctttcctcccttctacccatttttgttataattatttagaattaatcatacagttccctttcattcatccaagctattactctgttgcaacaaaggtcaggatgaaaagggcagcgcggttggcagctccatatttttacttcctcttggtgtaaagttttactgaataaagggagtgctacagtgagacgctcagacgaggggtaaataggaaatgagccagtgcccagctgggctcagcctggtccactccacattgccagccatcagactttctagaaaacacaggctgaaggcacccctgctgcctctcaaactctccgaggttttttgttttgttttgtgtgtgtgtgtgtgtgtgtgtgtgtgtgtgtggtgcaatgctcacacatgaagaaggccaaacctcaagctttcttcacgtacctgtacatttcagtctctgaaagaaagcactgtctactacgaatgttttattctgccatctgcctggtggaaattactgagggaaacagctggttcagcacttccgccactagcttttataaaggaagaaccaaagaactgttcctcatcagttgtgaagccta

SEQ ID NO: 11 - protein, 5 aa, synthetic construct
KRHRK

SEQ ID NO: 12 - protein, 5 aa, synthetic construct
RRRRR

SEQ ID NO: 13 - protein, 6 aa, synthetic construct
RRLARR

SEQ ID NO: 14 - protein, 11 aa, synthetic construct
KKAKAAAKPKK

SEQ ID NO: 15 - protein, 9 aa, synthetic construct
KKDGKKRKR

SEQ ID NO: 16 - protein, 5 aa, synthetic construct
KKKLK

SEQ ID NO: 17 - protein, 6 aa, synthetic construct
KKRIRK

SEQ ID NO: 18 - protein, 5 aa, synthetic construct
RKKSK

SEQ ID NO: 19 - protein, 5 aa, synthetic construct
KKPKK

SEQ ID NO: 20 - protein, 7 aa, synthetic construct
WGREERQ

SEQ ID NO: 21 - protein, 5 aa, synthetic construct
NTQIH

SEQ ID NO: 22 - protein, 7 aa, synthetic construct
WNNKTPH

SEQ ID NO: 23 - skipped

SEQ ID NO: 24 - protein, 5 aa, synthetic construct
VNRWS

SEQ ID NO: 25 - protein, 8 aa, synthetic construct
  VARIANT 1: note = X can be any Amnio Acid
  VARIANT 5..6: note = X can be any Amino Acid
  VARIANT 8: note = X can be any Amino Acid
XBBBXXBX

SEQ ID NO: 26 - protein, 8 aa, synthetic construct
ARKKAAKA

SEQ ID NO: 27-28 - skipped (2 entries)

SEQ ID NO: 29 - protein, 10 aa, synthetic construct
WEPSRPFPVD

SEQ ID NO: 30 - protein, 17 aa, synthetic construct
HRRTRKAPKRIRLPHIR

SEQ ID NO: 31 - protein, 17 aa, synthetic construct
KRTGQYKLGSKTGPGQK

SEQ ID NO: 32 - protein, 4 aa, synthetic construct
KKTK

SEQ ID NO: 33 - protein, 9 aa, synthetic construct
KLRSQLVKK

SEQ ID NO: 34 - protein, 9 aa, synthetic construct
RRRCGQKKK

SEQ ID NO: 35 - skipped

SEQ ID NO: 36 - protein, 17 aa, synthetic construct
RIQNLLKITNLRIKFVK

SEQ ID NO: 37 - protein, 11 aa, synthetic construct
KKEKDIMKKTI

SEQ ID NO: 38-39 - skipped (2 entries)

SEQ ID NO: 40 - protein, 4 aa, synthetic construct
RGDS

SEQ ID NO: 41 - protein, 11 aa, synthetic construct
TTVVNPKYEGK

SEQ ID NO: 42 - protein, 11 aa, synthetic construct
ERMSQIKRLLS

SEQ ID NO: 43 - protein, 7 aa, synthetic construct
WRHRARS

SEQ ID NO: 44 - protein, 6 aa, synthetic construct
GFOGER

SEQ ID NO: 45 - protein, 5 aa, synthetic construct
LFDLM

SEQ ID NO: 46 - protein, 7 aa, synthetic construct
WGREERQ

SEQ ID NO: 47 - protein, 7 aa, synthetic construct
QSTEKRG

SEQ ID NO: 48 - protein, 5 aa, synthetic construct
LPNTG

SEQ ID NO: 49 - skipped

SEQ ID NO: 50 - protein, 4 aa, synthetic construct
PPSY

SEQ ID NO: 51 - protein, 7 aa, synthetic construct
FEDNFVP

SEQ ID NO: 52 - protein, 4 aa, synthetic construct
YIRV

SEQ ID NO: 53 - protein, 4 aa, synthetic construct
YADW

SEQ ID NO: 54 - protein, 4 aa, synthetic construct
YTQV

SEQ ID NO: 55 - protein, 6 aa, synthetic construct
KKRPKP

SEQ ID NO: 56 - protein, 5 aa, synthetic construct
SSDDE

SEQ ID NO: 57 - protein, 5 aa, synthetic construct
RRASS

SEQ ID NO: 58 - protein, 8 aa, synthetic construct
  VARIANT 2..3: note = X can be any Amino Acid
  VARIANT 6..7: note = X can be any Amino Acid
YXXLYXXL

SEQ ID NO: 59 - protein, 5 aa, synthetic construct
LPLTG

SEQ ID NO: 60 - protein, 5 aa, synthetic construct
LAFTG

SEQ ID NO: 61 - protein, 7 aa, synthetic construct
ASSLNIA

SEQ ID NO: 62 - protein, 15 aa, synthetic construct
KKEEEKKEEEKKEEE

SEQ ID NO: 63 - protein, 7 aa, synthetic construct
LIFHKEQ

SEQ ID NO: 64 - protein, 10 aa, synthetic construct
KFNKPFVFLI

SEQ ID NO: 65 - protein, 7 aa, synthetic construct
QPEHSST

SEQ ID NO: 66 - protein, 7 aa, synthetic construct
EYHHYNK

SEQ ID NO: 67 - skipped

SEQ ID NO: 68 - protein, 6 aa, synthetic construct
GEKGEP

SEQ ID NO: 69 - protein, 5 aa, synthetic construct
KTKKK

SEQ ID NO: 70 - protein, 6 aa, synthetic construct
KALKKK

SEQ ID NO: 71 - protein, 5 aa, synthetic construct
KGKKK

SEQ ID NO: 72 - protein, 6 aa, synthetic construct
CSVTCG

SEQ ID NO: 73 - skipped

SEQ ID NO: 74 - protein, 10 aa, synthetic construct
YKYNLNGRES

SEQ ID NO: 75 - protein, 4 aa, synthetic construct
YRSL

SEQ ID NO: 76 - protein, 10 aa, synthetic construct
KGGKKKKKKK

SEQ ID NO: 77 - protein, 11 aa, synthetic construct
KKKQYTSIHHG

SEQ ID NO: 78 - protein, 4 aa, synthetic construct
KDEL

SEQ ID NO: 79 - protein, 10 aa, synthetic construct
LADQDYTKTA

SEQ ID NO: 80 - protein, 7 aa, synthetic construct
KKKYKLK

SEQ ID NO: 81 - protein, 6 aa, synthetic construct
KKRKLE

SEQ ID NO: 82 - protein, 4 aa, synthetic construct
TRSK

SEQ ID NO: 83 - protein, 6 aa, synthetic construct
HRKRKR

SEQ ID NO: 84 - protein, 6 aa, synthetic construct
NKRKRK

SEQ ID NO: 85 - protein, 7 aa, synthetic construct
AEKSKKK

SEQ ID NO: 86 - protein, 4 aa, synthetic construct
RKSK

SEQ ID NO: 87 - protein, 4 aa, synthetic construct
KRVK

SEQ ID NO: 88 - skipped

SEQ ID NO: 89 - protein, 11 aa, synthetic construct
LQQTPLHLAVI

SEQ ID NO: 90 - protein, 4 aa, synthetic construct
RRPR

SEQ ID NO: 91 - protein, 4 aa, synthetic construct
PRPR

SEQ ID NO: 92 - protein, 4 aa, synthetic construct
RPPP

SEQ ID NO: 93 - protein, 7 aa, synthetic construct
RKKRKGK

SEQ ID NO: 94 - protein, 9 aa, synthetic construct
PAAKRVKLD

SEQ ID NO: 95 - protein, 9 aa, synthetic construct
KLKIKRPVK

SEQ ID NO: 96 - protein, 7 aa, synthetic construct
PKKKRKV

SEQ ID NO: 97 - protein, 6 aa, synthetic construct
QRKRQK

SEQ ID NO: 98 - protein, 4 aa, synthetic construct
DSPE

SEQ ID NO: 99 - protein, 4 aa, synthetic construct
FQVT

SEQ ID NO: 100 - protein, 7 aa, synthetic construct
QSTEKRG

SEQ ID NO: 101 - protein, 6 aa, synthetic construct
RQGLID

SEQ ID NO: 102 - protein, 4 aa, synthetic construct
RKKH

SEQ ID NO: 103 - protein, 12 aa, synthetic construct
GRKKRRQRRRPQ

SEQ ID NO: 104 - skipped

SEQ ID NO: 105 - protein, 12 aa, synthetic construct
KSVKKRSVSEIQ

SEQ ID NO: 106 - protein, 8 aa, synthetic construct
NRRKKRAL

SEQ ID NO: 107 - protein, 5 aa, synthetic construct
KFERQ

SEQ ID NO: 108 - protein, 4 aa, synthetic construct
VRGP

SEQ ID NO: 109 - protein, 4 aa, synthetic construct
NKDS

SEQ ID NO: 110 - protein, 4 aa, synthetic construct
NRDN

SEQ ID NO: 111 - protein, 4 aa, synthetic construct
ANNR

SEQ ID NO: 112 - protein, 4 aa, synthetic construct
YTRF

SEQ ID NO: 113 - protein, 4 aa, synthetic construct
GDAY

SEQ ID NO: 114 - protein, 4 aa, synthetic construct
LLEE

SEQ ID NO: 115 - protein, 9 aa, synthetic construct
RKKRRQRRR

SEQ ID NO: 116 - protein, 4 aa, synthetic construct
YKSL

SEQ ID NO: 117 - protein, 4 aa, synthetic construct
YENF

SEQ ID NO: 118 - protein, 4 aa, synthetic construct
FQDL

SEQ ID NO: 119 - protein, 5 aa, synthetic construct
YIGSR

SEQ ID NO: 120 - protein, 5 aa, synthetic construct
IKVAV

SEQ ID NO: 121 - protein, 7 aa, synthetic construct
LIRERTE

SEQ ID NO: 122 - protein, 7 aa, synthetic construct
LVEERTQ

SEQ ID NO: 123 - protein, 6 aa, synthetic construct
IITFTK

SEQ ID NO: 124 - protein, 5 aa, synthetic construct
ILFNK

SEQ ID NO: 125 - protein, 7 aa, synthetic construct
PIRTLSK

SEQ ID NO: 126 - protein, 10 aa, synthetic construct
YGNSPLHRFK

SEQ ID NO: 127 - protein, 6 aa, synthetic construct
FFQKDR

SEQ ID NO: 128 - protein, 4 aa, synthetic construct
KSRP

SEQ ID NO: 129 - skipped

SEQ ID NO: 130 - protein, 4 aa, synthetic construct
YMKM

SEQ ID NO: 131 - protein, 6 aa, synthetic construct
RSSSFG

SEQ ID NO: 132 - protein, 8 aa, synthetic construct
LKIRGRER

SEQ ID NO: 133 - protein, 8 aa, synthetic construct
LKIRGRKR

SEQ ID NO: 134 - protein, 9 aa, synthetic construct
HVIFKKVSR

SEQ ID NO: 135 - protein, 5 aa, synthetic construct
RGPRV

SEQ ID NO: 136 - protein, 8 aa, synthetic construct
RANVKHLK

SEQ ID NO: 137 - protein, 5 aa, synthetic construct
YPKAG

SEQ ID NO: 138 - protein, 5 aa, synthetic construct
YPRTG

SEQ ID NO: 139 - protein, 4 aa, synthetic construct
DDNN

SEQ ID NO: 140 - protein, 8 aa, synthetic construct
SAVTTVVN

SEQ ID NO: 141 - protein, 12 aa, synthetic construct
GRKKRRQRRRPQ

SEQ ID NO: 142 - protein, 12 aa, synthetic construct
KSVKKRSVSEIQ

SEQ ID NO: 143 - protein, 8 aa, synthetic construct
NRRKKRAL

SEQ ID NO: 144 - protein, 34 aa, synthetic construct
KRHRKLREKRHRKLRRRRRLKRHRKKRHRKLREK

SEQ ID NO: 145 - protein, 40 aa, synthetic construct
KRHRKGSSLREKRHRKLRRRRRLKRHRKKRHRKLREGGSK

SEQ ID NO: 146 - protein, 44 aa, synthetic construct
KRHRKREGSSLREKRHRKNDLRRRRRLKRHRKKRHRKLREGGSK

SEQ ID NO: 147 - protein, 44 aa, synthetic construct
KKPKKREGSSLREKRHRKNDLRRRRRLKRHRKKRHRKLREGGSK

SEQ ID NO: 148 - protein, 41 aa, synthetic construct
RRLARRGSSLREKRHRKLRRRRRLKKPKKKRHRKLREGGSK

SEQ ID NO: 149 - protein, 36 aa, synthetic construct
KRHRKLREKRHRKLREKRHRKLKRHRKKRHRKLREK

SEQ ID NO: 150 - protein, 40 aa, synthetic construct
KRHRKRILREKRHRKLREARKRHRKLKRHRKKRHRKLREK

SEQ ID NO: 151 - protein, 35 aa, synthetic construct
KRHRKKGKKKKGEKGKKKLKGKKKLRRRRRRRQRR

SEQ ID NO: 152 - protein, 43 aa, synthetic construct
KRHRKAPAPKGKKKKGEKGKKKLKGKKKLKPKPRRRRRRRQRR

SEQ ID NO: 153 - protein, 41 aa, synthetic construct
KRHRKGGSGGKGKKKKGEKGKKKLKGKKKLARRRRRRRQRR

SEQ ID NO: 154 - protein, 34 aa, synthetic construct
KRHRKLREKRHRKRRRRRRRKRHRKLREKRRQRR

SEQ ID NO: 155 - protein, 30 aa, synthetic construct
KRHRKKRHRKKRVKKKRHRKRRRRRRDSLL

SEQ ID NO: 156 - protein, 35 aa, synthetic construct
KRHRKKRHRKYQKRVKKKRHRKSSSRRRRRRDSLL

SEQ ID NO: 157 - protein, 32 aa, synthetic construct
KRHRKKKEEEKKEEEKKEEEKRRRRRRRQRRR

SEQ ID NO: 158 - protein, 36 aa, synthetic construct
KRHRKWRKKEEEKKEEEKKEEEKRIRRRRRRRQRRR

SEQ ID NO: 159 - protein, 29 aa, synthetic construct
KRHRKRGDKRHRKRRRRRKRHRKTPHKKK

SEQ ID NO: 160 - protein, 33 aa, synthetic construct
KRHRKFIRGDKRHRKRRRRRKRHRKLATPHKKK

SEQ ID NO: 161 - protein, 36 aa, synthetic construct
KRHRKRGDKRHRKRRRRRKRHRKGSSRNTPHQKKKK

SEQ ID NO: 162 - protein, 33 aa, synthetic construct
KRHRKRGDKRHRKLKRHRKRRRRKRHRKTPHKK

SEQ ID NO: 163 - protein, 30 aa, synthetic construct
KRHRKRGDKRHRKKRHRKKRHRKRGDKKTK

SEQ ID NO: 164 - protein, 32 aa, synthetic construct
KRHRKRGDKKRKKKKRGDKKRRRRRKKKPPSY

SEQ ID NO: 165 - protein, 32 aa, synthetic construct
KRHRKRKRKRKRRRRRKKKRASSLNIAKRRRR

SEQ ID NO: 166 - protein, 32 aa, synthetic construct
KRKKRKGKRLKRRREKRHRKRASSLNIAKKKK

SEQ ID NO: 167 - protein, 32 aa, synthetic construct
KRKKRRLKRKRKRRRRREKRHRKRRRQRRRKK

SEQ ID NO: 168 - protein, 31 aa, synthetic construct
KRKKRRKRKRRRRRKRHRKLRERKRRLREKK

SEQ ID NO: 169 - protein, 34 aa, synthetic construct
KRKNGRKRKRKKRHRKKKKRRRRKRHRKNGRKKK

SEQ ID NO: 170 - protein, 29 aa, synthetic construct
KRHRKWRHRARSKRHRKKKKKKRKKRKGK

SEQ ID NO: 171 - protein, 31 aa, synthetic construct
KRHRKRGDKRHRKKKKNRRKKRALRKKRKGK

SEQ ID NO: 172 - protein, 38 aa, synthetic construct
KKRKRGGKTKKKAKKALKKKKKGKKKKRRRRKKAAPKK

SEQ ID NO: 173 - protein, 43 aa, synthetic construct
KKKAYPKALKKPKKKKKAYPKALKRRRRRKNRRKKRALKRHRK

SEQ ID NO: 174 - protein, 39 aa, synthetic construct
KTRSKKKKKRGDKKKKNRRKKRALNTQIHKKKKKAAPKK

SEQ ID NO: 175 - protein, 34 aa, synthetic construct
KGKKKKGEKGKKKLKGKKKLRRRRRSPKKRRQRR

SEQ ID NO: 176 - protein, 34 aa, synthetic construct; identical to SEQ ID NO: 144

SEQ ID NO: 177 - protein, 36 aa, synthetic construct; identical to SEQ ID NO: 149

SEQ ID NO: 178 - protein, 35 aa, synthetic construct; identical to SEQ ID NO: 151

SEQ ID NO: 179 - protein, 34 aa, synthetic construct; identical to SEQ ID NO: 154

SEQ ID NO: 180 - protein, 30 aa, synthetic construct; identical to SEQ ID NO: 155

SEQ ID NO: 181 - protein, 32 aa, synthetic construct; identical to SEQ ID NO: 157

SEQ ID NO: 182 - protein, 36 aa, synthetic construct
KRHRKQSKKEEEKKEEEKKEEEKNQRRRRRRRQRRR

SEQ ID NO: 183 - protein, 29 aa, synthetic construct
KRHRKRGDKRHRKRRRRRKRHRKTPHKKK

SEQ ID NO: 184 - protein, 33 aa, synthetic construct; identical to SEQ ID NO: 162

SEQ ID NO: 185 - protein, 30 aa, synthetic construct; identical to SEQ ID NO: 163

SEQ ID NO: 186 - protein, 32 aa, synthetic construct; identical to SEQ ID NO: 164

SEQ ID NO: 187 - protein, 38 aa, synthetic construct
KRHRKGGSRGDKKRKKKKRGDSSSKKRRRRRKKKPPSY

SEQ ID NO: 188 - protein, 32 aa, synthetic construct; identical to SEQ ID NO: 165

SEQ ID NO: 189 - protein, 32 aa, synthetic construct; identical to SEQ ID NO: 166

SEQ ID NO: 190 - protein, 32 aa, synthetic construct; identical to SEQ ID NO: 167

SEQ ID NO: 191 - protein, 31 aa, synthetic construct; identical to SEQ ID NO: 168

SEQ ID NO: 192 - protein, 34 aa, synthetic construct; identical to SEQ ID NO: 169

SEQ ID NO: 193 - protein, 40 aa, synthetic construct
KRKWRNGRKRKRQKRHRKKKKRARRRRKRHRKNGRKHKKK

SEQ ID NO: 194 - protein, 29 aa, synthetic construct
KRHRKWRHRARSKRHRKKKPKKRKKRKGK

SEQ ID NO: 195 - protein, 36 aa, synthetic construct
KRHRKPKPRIWRHRARSRDKRHRKKKPKKRKKRKGK

SEQ ID NO: 196 - DNA, 26 bp, synthetic construct
  source 1..26: mol_type = other DNA
aaaannnnnnnaaaannnnnnnaaaa

SEQ ID NO: 197 - DNA, 26 bp, synthetic construct
  source 1..26: mol_type = other DNA
ttttnnnnnnnttttnnnnnnntttt

SEQ ID NO: 198 - DNA, 16 bp, synthetic construct
  source 1..16: mol_type = other DNA
awwrtaannwwgnnnc

SEQ ID NO: 199 - DNA, 18 bp, synthetic construct
  source 1..18: mol_type = other DNA
rnynncnngyngktnyny

SEQ ID NO: 200 - DNA, 15 bp, synthetic construct
  source 1..15: mol_type = other DNA
gtnwayattnatnnr

SEQ ID NO: 201 - DNA, 12 bp, synthetic construct
  source 1..12: mol_type = other DNA
aacttgtttatt

SEQ ID NO: 202 - DNA, 20 bp, synthetic construct
  source 1..20: mol_type = other DNA
ttctttttttttctttttct

SEQ ID NO: 203 - DNA, 20 bp, synthetic construct
  source 1..20: mol_type = other DNA
ttatttttttttctttgcct

SEQ ID NO: 204 - DNA, 12 bp, synthetic construct
  source 1..12: mol_type = other DNA
gccgccattttg

SEQ ID NO: 205 - DNA, 27 bp, synthetic construct
  source 1..27: mol_type = other DNA
tggggagggtggggagggtggggaagg

SEQ ID NO: 206 - DNA, 25 bp, synthetic construct
  source 1..25: mol_type = other DNA
cgaaaggaattggaataaaaatttc

SEQ ID NO: 207 - DNA, 10 bp, synthetic construct
  source 1..10: mol_type = other DNA
rttgcgyaay

SEQ ID NO: 208 - DNA, 14 bp, synthetic construct
  source 1..14: mol_type = other DNA
ccgcgnggnggcag

(19) United States
(12) Patent Application Publication
(10) Pub. No.: US 2026/0152759 A1
(43) Pub. Date: Jun. 4, 2026

(54) SCAFFOLD MATRIX ATTACHMENT REGIONS FOR GENE THERAPY

(71) Applicant: SUMMATION BIO, INC., Somerville, MA (US)

(72) Inventors: Adarsha Koirala, Framingham, MA (US); Almudena Pacheco-Garcia, Carlisle, MA (US); Marc A. T. Muskavitch, Millis, MA (US); Karen Ellen Petra De Ceunynck, Jamaica Plain, MA (US)

(21) Appl. No.: 18/294,828

(22) PCT Filed: Aug. 2, 2022
(86) PCT No.: PCT/US2022/039187

§ 371 Date: Feb. 2, 2024

Related U.S. Application Data
(60) Provisional application No. 63/228,712, filed on Aug. 3, 2021.

(51) Int. Cl. C12N 15/85; A61K 48/00

(52) U.S. Cl. CPC C12N 15/85; A61K 48/0066; C12N 2830/46

(57) ABSTRACT

The present disclosure provides, among other things, methods and compositions in which a nucleic acid sequence includes a mammalian scaffold matrix attachment region (S/MAR) operably linked with a coding sequence. The present disclosure provides S/MARs useful in expression vectors (e.g., for use in gene therapy approaches) to achieve a particular, improved, or target level, duration, and/or tissue specificity of expression of an operably linked coding sequence.

### CROSS REFERENCE TO RELATED APPLICATIONS

This application claims the benefit of U.S. Provisional Application No. 63/228,712 filed Aug. 3, 2021, the contents of which are hereby incorporated herein in their entirety.

### BACKGROUND

Many medical conditions can be treated by gene therapy. Gene therapy includes delivery of nucleic acids that encode and/or express therapeutic products to subjects in need thereof. Factors impacting the efficacy of gene therapy include the level, duration, and tissue specificity of expression.

### SUMMARY

The present disclosure provides, among other things, methods and compositions in which a nucleic acid sequence includes a mammalian scaffold matrix attachment region (S/MAR) operably linked with a coding sequence. S/MARs are nucleic acid sequences that participate in biological interactions that impact expression of associated coding sequences. Putative human S/MARs have been identified based on bioinformatic approaches. However, expression characteristics of individual S/MARs or sets of S/MARs, including expression level, duration, and tissue specificity conferred by S/MARs, are not fully understood. Moreover, the putative S/MARs identified based on bioinformatic approaches are highly diverse in sequence, and for at least this reason the characteristics of putative S/MARs cannot be inferred from sequence similarity with the few S/MARs that have been studied alone. The present disclosure identifies certain S/MARs useful in expression vectors (e.g., for use in gene therapy approaches) to achieve a particular, improved, or target level, duration, and/or tissue specificity of expression of an operably linked coding sequence, e.g., where improvement is determined by comparison to a reference such as a reference construct that does not include the S/MAR or a reference construct that includes a control S/MAR such as hu1FNb.

In certain embodiments, an expression vector of the present disclosure is associated with a mini-nucleosome core protein, e.g., as described in PCT Publication WO 2020/097235, which is incorporated herein by reference in its entirety and particularly with respect to mini-nucleosome core proteins and sequences thereof. In certain embodiments, an expression vector of the present disclosure is associated with a modified mini-nucleosome core protein, e.g., as described in International Application No. PCT/US21/26917, which is incorporated herein by reference in its entirety and particularly with respect to modified mini-nucleosome core proteins and sequences thereof.

In at least one aspect, the present disclosure provides an expression vector including an S/MAR, where the S/MAR has at least 80% sequence identity with one of SEQ ID NOs: 1-10. In various embodiments, the S/MAR has at least 80% sequence identity with one of SEQ ID NOs: 1-6. In at least one aspect, the present disclosure provides an expression vector including an S/MAR, where the S/MAR has at least 80% sequence identity with an S/MAR of Table 3.

In various embodiments, an S/MAR includes (i) an origin of replication (OriC) sequence, (ii) AT richness, (iii) kinked DNA, (iv) curved DNA, (v) TG richness, (vi) a MAR signature, (vii) a Topoisomerase-II site, or (viii) a combination thereof. In various embodiments, the S/MAR includes binding sites for (i) BRCA1, (ii) BRIGHT, (iii) SMAR1, (iv) CEBPB, (v) CUX1/CDP, (vi) CTCF, (vii) Fast1/FOXH1, (viii) HoxC11, (ix) Ku autoantigen, (x) NMP4, (xi) Mut-p53, (xii) SAF-A/hnRNPU, (xiii) SATB1, (xiv) YY1, or (xv) a combination thereof. In various embodiments, the S/MAR includes binding sites for (i) BRIGHT, (ii) CEBPB, (iii) CTCF, (iv) Fast1/FOXH1, (v) Ku autoantigen, (vi) NMP4, (vii) Mut p53, (viii) SAF-A/hnRNPU, (ix) SATB1, (x) YY1, or (xi) a combination thereof. In various embodiments, the S/MAR is operably linked with a coding sequence. In various embodiments, the S/MAR is upstream of the coding sequence, optionally where the S/MAR is no more than 2 kb, 1 kb, 500 bp, or 100 bp upstream of a transcription start site of the coding sequence. In various embodiments, the S/MAR is downstream of the coding sequence, optionally where the S/MAR is no more than 2 kb, 1 kb, 500 bp, or 100 bp downstream of the coding sequence. In various embodiments, the S/MAR is present in an intron of the coding sequence.

In various embodiments, an expression vector is a circular nucleic acid, optionally where the expression vector is a plasmid. In various embodiments, an expression vector is a linear nucleic acid. In various embodiments, an expression vector is associated with an engineered polypeptide including (i) a nucleic acid binding domain and (ii) a nucleic acid release domain, a nuclear localization signal, a stability domain, an oligomerization domain, a targeting domain, or a combination thereof, optionally where the engineered polypeptide is a mini-nucleosome core protein. In various embodiments, an expression vector is a viral genome and/or is present in a viral vector.

In at least one aspect, the present disclosure provides a method of expressing a coding sequence in liver cells, retinal cells, skeletal muscle cells, neuronal cells, central nervous system cells, or a combination thereof, the method including delivering an expression vector to the liver cells, retinal cells, skeletal muscle cells, neuronal cells, central nervous system cells, or combination thereof, where the expression vector including an S/MAR operably linked with a coding sequence, where the S/MAR has at least 80% sequence identity with one of SEQ ID NOs: 1-10, optionally where the delivering includes administering the expression vector to a subject, and optionally where the subject is a human. In various embodiments, the S/MAR has at least 80% sequence identity with one of SEQ ID NOs: 1-6. In at least one aspect, the present disclosure provides a method of expressing a coding sequence in liver cells, retinal cells, skeletal muscle cells, neuronal cells, central nervous system cells, or a combination thereof, the method including delivering an expression vector to the liver cells, retinal cells, skeletal muscle cells, neuronal cells, central nervous system cells, or combination thereof, where the expression vector including an S/MAR operably linked with a coding sequence, where the S/MAR has at least 80% sequence identity with an S/MAR of Table 3, optionally where the delivering includes administering the expression vector to a subject, and optionally where the subject is a human.

In various embodiments, an expression of the coding sequence in the liver cells, retinal cells, skeletal muscle cells, neuronal cells, central nervous system cells, or combination thereof is characterized by (i) increased expression as compared to a reference, (ii) increased duration of expression as compared to a reference, (iii) comparable expression as compared to a reference, (iv) comparable duration of expression as compared to a reference, or (v) a combination thereof. In various embodiments, an expression vector is associated with an engineered polypeptide including (i) a nucleic acid binding domain and (ii) a nucleic acid release domain, a nuclear localization signal, a stability domain, an oligomerization domain, a targeting domain, or a combination thereof, optionally where the engineered polypeptide is a mini-nucleosome core protein. In various embodiments, an expression vector is a viral genome and/or is present in a viral vector.

In at least one aspect, the present disclosure provides a method of producing an expression vector composition including contacting (i) an expression vector including an S/MAR operably linked with a coding sequence, where the S/MAR has at least 80% sequence identity with one of SEQ ID NOs: 1-10, with (ii) an engineered polypeptide including (a) a nucleic acid binding domain and (b) a nucleic acid release domain, a nuclear localization signal, a stability domain, an oligomerization domain, a targeting domain, or a combination thereof, optionally where the engineered polypeptide is a mini-nucleosome core protein. In various embodiments, an S/MAR has at least 80% sequence identity with one of SEQ ID NOs: 1-6. In at least one aspect, the present disclosure provides a method of producing an expression vector composition including contacting (i) an expression vector including an S/MAR operably linked with a coding sequence, where the S/MAR has at least 80% sequence identity with an S/MAR of Table 3, with (ii) an engineered polypeptide including (a) a nucleic acid binding domain and (b) a nucleic acid release domain, a nuclear localization signal, a stability domain, an oligomerization domain, a targeting domain, or a combination thereof, optionally where the engineered polypeptide is a mini-nucleosome core protein.

In various embodiments, an S/MAR includes (i) an origin of replication (OriC) sequence, (ii) AT richness, (iii) kinked DNA, (iv) curved DNA, (v) TG richness, (vi) a MAR signature, (vii) a Topoisomerase-II site, or (viii) a combination thereof. In various embodiments, an S/MAR includes binding sites for (i) BRCA1, (ii) BRIGHT, (iii) SMAR1, (iv) CEBPB, (v) CUX1/CDP, (vi) CTCF, (vii) Fast1/FOXH1, (viii) HoxC11, (ix) Ku autoantigen, (x) NMP4, (xi) Mut-p53, (xii) SAF-A/hnRNPU, (xiii) SATB1, (xiv) YY1, or (xv) a combination thereof. In various embodiments, an S/MAR includes binding sites for (i) BRIGHT, (ii) CEBPB, (iii) CTCF, (iv) Fast1/FOXH1, (v) Ku autoantigen, (vi) NMP4, (vii) Mut p53, (viii) SAF-A/hnRNPU, (ix) SATB1, (x) YY1, or (xi) a combination thereof.

In various embodiments, an S/MAR is upstream of the coding sequence, optionally where the S/MAR is no more than 2 kb, 1 kb, 500 bp, or 100 bp upstream of a transcription start site of the coding sequence. In various embodiments, an S/MAR is downstream of the coding sequence, optionally where the S/MAR is no more than 2 kb, 1 kb, 500 bp, or 100 bp downstream of the coding sequence. In various embodiments, an S/MAR is present in an intron of the coding sequence. In various embodiments, an expression vector is a circular nucleic acid, optionally where the expression vector is a plasmid. In various embodiments, an expression vector is a linear nucleic acid.

### Definitions

About: The term “about,” when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system (e.g., a plurality of cells, in vivo, in vitro, or ex vivo, e.g., where the plurality of cells includes one or more target and/or host cells) to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or include, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Antibody: As used herein, the term “antibody” refers to a polypeptide that includes one or more canonical immunoglobulin sequence elements sufficient to confer specific binding to a particular antigen (e.g., a heavy chain variable domain, a light chain variable domain, and/or one or more CDRs). Thus, the term antibody includes, without limitation, human antibodies, non-human antibodies, synthetic and/or engineered antibodies, fragments thereof, and agents including the same. Antibodies can be naturally occurring immunoglobulins (e.g., generated by an organism reacting to an antigen). Synthetic, non-naturally occurring, or engineered antibodies can be produced by recombinant engineering, chemical synthesis, or other artificial systems or methodologies known to those of skill in the art.

As is well known in the art, typical human immunoglobulins are approximately 150 kD tetrameric agents that include two identical heavy (H) chain polypeptides (about 50 kD each) and two identical light (L) chain polypeptides (about 25 kD each) that associate with each other to form a structure commonly referred to as a “Y-shaped” structure. Typically, each heavy chain includes a heavy chain variable domain (VH) and a heavy chain constant domain (CH). The heavy chain constant domain includes three CH domains: CH1, CH2 and CH3. A short region, known as the “switch”, connects the heavy chain variable and constant regions. The “hinge” connects CH2 and CH3 domains to the rest of the immunoglobulin. Each light chain includes a light chain variable domain (VL) and a light chain constant domain (CL), separated from one another by another “switch.” Each variable domain contains three hypervariable loops known as “complement determining regions” (CDR1, CDR2, and CDR3) and four somewhat invariant “framework” regions (FR1, FR2, FR3, and FR4). In each VH and VL, the three CDRs and four FRs are arranged from amino-terminus to carboxy-terminus in the following order: FR1, CDR1, FR2, CDR2, FR3, CDR3, and FR4. The variable regions of a heavy and/or a light chain are typically understood to provide a binding moiety that can interact with an antigen. Constant domains can mediate binding of an antibody to various immune system cells (e.g., effector cells and/or cells that mediate cytotoxicity), receptors, and elements of the complement system. Heavy and light chains are linked to one another by a single disulfide bond, and two other disulfide bonds connect the heavy chain hinge regions to one another, so that the dimers are connected to one another and the tetramer is formed. When natural immunoglobulins fold, the FR regions form the beta sheets that provide the structural framework for the domains, and the CDR loop regions from both the heavy and light chains are brought together in three-dimensional space so that they create a single hypervariable antigen binding site located at the tip of the Y structure.

In some embodiments, an antibody is a polyclonal, monoclonal, monospecific, or multispecific antibody (e.g., a bispecific antibody). In some embodiments, an antibody includes at least one light chain monomer or dimer, at least one heavy chain monomer or dimer, at least one heavy chain-light chain dimer, or a tetramer that includes two heavy chain monomers and two light chain monomers. Moreover, the term “antibody” can include (unless otherwise stated or clear from context) any art-known constructs or formats utilizing antibody structural and/or functional features including without limitation intrabodies, domain antibodies, antibody mimetics, Zybodies®, Fab fragments, Fab′ fragments, F(ab′)2 fragments, Fd′ fragments, Fd fragments, isolated CDRs or sets thereof, single chain antibodies, single-chain Fvs (scFvs), disulfide-linked Fvs (sdFv), polypeptide-Fc fusions, single domain antibodies (e.g., shark single domain antibodies such as IgNAR or fragments thereof), cameloid antibodies, camelized antibodies, masked antibodies (e.g., Probodies®), affybodies, anti-idiotypic (anti-Id) antibodies (including, e.g., anti-anti-Id antibodies), Small Modular ImmunoPharmaceuticals (“SMIPs™”), single chain or Tandem diabodies (TandAb®), VHHs, Anticalins®, Nanobodies® minibodies, BiTE®s, ankyrin repeat proteins or DARPINs®, Avimers®, DARTs, TCR-like antibodies, Adnectins®, Affilins®, Trans-bodies®, Affibodies®, TrimerX®, MicroProteins, Fynomers®, Centyrins®, and KALBITOR®s, CARs, engineered TCRs, and antigen-binding fragments of any of the above.

In various embodiments, an antibody includes one or more structural elements recognized by those skilled in the art as a complementarity determining region (CDR) or variable domain. In some embodiments, an antibody can be a covalently modified (“conjugated”) antibody (e.g., an antibody that includes a polypeptide including one or more canonical immunoglobulin sequence elements sufficient to confer specific binding to a particular antigen, where the polypeptide is covalently linked with one or more of a therapeutic agent, a detectable moiety, another polypeptide, a glycan, or a polyethylene glycol molecule). In some embodiments, antibody sequence elements are humanized, primatized, chimeric, etc, as is known in the art.

An antibody including a heavy chain constant domain can be, without limitation, an antibody of any known class, including but not limited to, IgA, secretory IgA, IgG, IgE and IgM, based on heavy chain constant domain amino acid sequence (e.g., alpha (α), delta (δ), epsilon (ε), gamma (γ) and mu (μ)). IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. As used herein, a “light chain” can be of a distinct type, e.g., kappa (κ) or lambda (λ), based on the amino acid sequence of the light chain constant domain. In some embodiments, an antibody has constant region sequences that are characteristic of mouse, rabbit, primate, or human immunoglobulins. Naturally-produced immunoglobulins are glycosylated, typically on the CH2 domain. As is known in the art, affinity and/or other binding attributes of Fc regions for Fc receptors can be modulated through glycosylation or other modification. In some embodiments, an antibody may lack a covalent modification (e.g., attachment of a glycan) that it would have if produced naturally. In some embodiments, antibodies produced and/or utilized in accordance with the present invention include glycosylated Fc domains, including Fc domains with modified or engineered such glycosylation.

Antibody fragment: As used herein, an “antibody fragment” refers to a portion of an antibody or antibody agent as described herein, and typically refers to a portion that includes an antigen-binding portion or variable region thereof Δn antibody fragment can be produced by any means. For example, in some embodiments, an antibody fragment can be enzymatically or chemically produced by fragmentation of an intact antibody or antibody agent. Alternatively, in some embodiments, an antibody fragment can be recombinantly produced (i.e., by expression of an engineered nucleic acid sequence. In some embodiments, an antibody fragment can be wholly or partially synthetically produced. In some embodiments, an antibody fragment (particularly an antigen-binding antibody fragment) can have a length of at least about 50, 60, 70, 80, 90, 100, 110, 120, 130, 140, 150, 160, 170, 180, 190 amino acids or more, in some embodiments at least about 200 amino acids.

Associated with: Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Agent: As used herein, the term “agent,” may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that includes a polymer. In some embodiments, the term may refer to a compound or entity that includes one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: In its broadest sense, as used herein, “amino acid” refers to any compound and/or substance that can be incorporated into a polypeptide chain, e.g., through formation of one or more peptide bonds. In some embodiments, an amino acid has the general structure H₂N—C(H)R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Between: As used herein, the term “between” refers to content that falls between indicated upper and lower, or first and second, boundaries, inclusive of the boundaries.

Biological Sample: As used herein, the term “biological sample” typically refers to a sample obtained or derived from a biological source (e.g., a tissue, organism, system or cell culture) of interest. In some embodiments, a biological source is or includes an organism, such as an animal or human. In some embodiments, a biological sample is or includes biological tissue or fluid. In some embodiments, a biological sample can be or include cells, tissue, or bodily fluid. In some embodiments, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or broncheoalveolar lavages, aspirates, scrapings, or bone marrow. In some embodiments, a biological sample is or includes cells obtained from a single subject or from a plurality of subjects. A sample can be a “primary sample” obtained directly from a biological source, or can be a “processed sample” (e.g., a sample prepared from a primary sample, e.g., by a process such as isolation, e.g., of mRNA, DNA, or protein, by a process that modifies the primary sample's chemical structure, and/or by a process that produces a new or different composition that represents one or more components or properties of the primary sample). A biological sample can also be referred to as a “sample.”

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, β-helix character, β-sheet character, coiled-coil character, random coil character, etc.), and/or by a particular functional feature (e.g., binding activity, enzymatic activity, folding activity, signaling activity, etc.). In some embodiments, a domain is or includes a characteristic portion or characteristic sequence element.

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence. In some embodiments, an engineered polynucleotide includes a domain-encoding sequence regulatory sequence that is found in nature in operative association with a first sequence but not in operative association with a second sequence, is linked by the hand of man so that it is operatively associated with the second sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Gene: As used herein, the term “gene” refers to a DNA sequence that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., a promoter). A gene can be endogenous or non-endogenous in a particular context, e.g., a cell. A gene can be a transgene.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Heterologous: As used herein, a first nucleic acid sequence is “heterologous” to a second nucleic acid sequence if the first nucleic acid sequence is not operatively linked with the second nucleic acid sequence in nature. By extension, a polypeptide is “heterologous” to an expression control sequence if it is encoded by nucleic acid sequence heterologous the promoter.

Host cell, target cell: As used herein, “host cell” refers to a cell into which exogenous nucleic acid (recombinant or otherwise), such as a transgene, has been introduced. Those of skill in the art appreciate that a “host cell” can be the cell into which the exogenous nucleic acid was initially introduced and/or progeny or copies, perfect or imperfect, thereof. In some embodiments, a host cell includes one or more transgenes. In some embodiments, an intended or potential host cell can be referred to as a target cell.

Improve, increase, inhibit, decrease, or reduce: As used herein, the terms “improve,” “increase,” “inhibit,” “decrease,” “reduce,” or grammatical equivalents thereof, indicate values that indicate qualitative or quantitative difference from a baseline or other reference. In some embodiments, an appropriate reference may be or include a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference may be or include a measurement in comparable system known or expected to respond in a particular way, in presence of the relevant agent or treatment.

Nucleic acid: As used herein, in its broadest sense, “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain including individual nucleic acid residues. In some embodiments, a “nucleic acid” is or includes RNA; in some embodiments, a “nucleic acid” is or includes DNA. In some embodiments, a nucleic acid is, includes, or consists of one or more natural nucleic acid residues. In some embodiments, a nucleic acid is, includes, or consists of one or more nucleic acid analogs. In some embodiments, a nucleic acid analog differs from a nucleic acid in that it does not utilize a phosphodiester backbone. For example, in some embodiments, a nucleic acid is, includes, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, includes, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, includes, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid includes one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence including at least one element that encodes, or is the complement of a sequence that encodes, a polypeptide. In some embodiments, a nucleic acid has enzymatic activity.

Operably linked: As used herein, “operably linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. Those of skill in the art will appreciate that such control can be indirect, e.g., where a regulatory sequence recruits factors that impact expression of a coding sequence or where a regulatory sequence recruits factors or otherwise influences activity of a promoter that impacts or derives expression of a coding sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polypeptide: As used herein, “polypeptide” refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may include or consist of natural amino acids, non-natural amino acids, or both. In some embodiments, a polypeptide may include or consist of only natural amino acids or only non-natural amino acids. In some embodiments, a polypeptide may include D-amino acids, L-amino acids, or both. In some embodiments, a polypeptide may include only D-amino acids. In some embodiments, a polypeptide may include only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, phosphorylation, glycosylation, glycation, sulfation, mannosylation, nitrosylation, acylation, palmitoylation, prenylation, pegylation, etc., including combinations thereof. In some embodiments, a polypeptide may be cyclic, and/or may include a cyclic portion. In some embodiments, a polypeptide is not cyclic and/or does not include any cyclic portion. In some embodiments, a polypeptide is linear. In some embodiments, a polypeptide may be or include a stapled polypeptide. In some embodiments, the term “polypeptide” may be appended to a name of a reference polypeptide, activity, or structure; in such instances, it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence similarity (e.g., homology) or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence similarity (e.g., homology) or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or include a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may include or consist of a fragment of a parent polypeptide. In some embodiments, a useful polypeptide as may include or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: As used herein, “prevent” or “prevention,” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked with, expression control sequences such as one or more S/MAR, enhancer, and/or repressor sequences, e.g., where the promoter and expression control sequence (e.g., the S/MAR) together control and/or contribute to expression of a coding sequence to which they are each individually, and together, operably linked. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In various embodiments a promoter is identified as a sequence including a given number of nucleotides that are immediately upstream of a transcription start site of a coding sequence of a known nucleic acid sequence (e.g., a genomic sequence), e.g., where the number of nucleotides can be 100 bp, 200 bp, 500 bp, 1 kb, 2 kb, 3 kb, 4 kb, or 5 kb. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, NY, 1989). Those of skill in the art are familiar with a wide variety of known promoters and various uses and/or characteristics thereof.

Recombinant: As used herein, “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” typically refers to an aliquot of material obtained or derived from a source of interest. In some embodiments, a source of interest is a biological or environmental source. In some embodiments, a sample is a “primary sample” obtained directly from a source of interest. In some embodiments, as will be clear from context, the term “sample” refers to a preparation that is obtained by processing of a primary sample (e.g., by removing one or more components of and/or by adding one or more agents to a primary sample). Such a “processed sample” can include, for example cells, nucleic acids, or proteins extracted from a sample or obtained by subjecting a primary sample to techniques such as amplification or reverse transcription of nucleic acids, isolation and/or purification of certain components, etc.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantial sequence similarity: The phrase “substantial sequence similarity” is used herein to refer to a comparison between amino acid or nucleic acid sequences. As will be appreciated by those of ordinary skill in the art, two sequences are generally considered to be “substantially similar” if they contain a conservative amino acid substitution in corresponding positions. A conservative substitution is one in which an amino acid has been replaced by a non-identical residue having appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a conservative substitution. Typical amino acid categorizations are summarized in Tables 1 and 2 below:

TABLE 1

| | | | | | |
|---|---|---|---|---|---|
| Alanine | Ala | A | Nonpolar | neutral | 1.8 |
| Arginine | Arg | R | Polar | positive | −4.5 |
| Asparagine | Asn | N | Polar | neutral | −3.5 |
| Aspartic acid | Asp | D | Polar | negative | −3.5 |
| Cysteine | Cys | C | Nonpolar | neutral | 2.5 |
| Glutamic acid | Glu | E | Polar | negative | −3.5 |
| Glutamine | Gln | Q | Polar | neutral | −3.5 |
| Glycine | Gly | G | Nonpolar | neutral | −0.4 |
| Histidine | His | H | Polar | positive | −3.2 |
| Isoleucine | Ile | I | Nonpolar | neutral | 4.5 |
| Leucine | Leu | L | Nonpolar | neutral | 3.8 |
| Lysine | Lys | K | Polar | positive | −3.9 |
| Methionine | Met | M | Nonpolar | neutral | 1.9 |
| Phenylalanine | Phe | F | Nonpolar | neutral | 2.8 |
| Proline | Pro | P | Nonpolar | neutral | −1.6 |
| Serine | Ser | S | Polar | neutral | −0.8 |
| Threonine | Thr | T | Polar | neutral | −0.7 |
| Tryptophan | Trp | W | Nonpolar | neutral | −0.9 |
| Tyrosine | Tyr | Y | Polar | neutral | −1.3 |
| Valine | Val | V | Nonpolar | neutral | 4.2 |

TABLE 2

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs,” Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying similar sequences, the programs mentioned above typically provide an indication of the degree of similarity. In some embodiments, two sequences are considered to be substantially similar if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are similar and/or identical over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues. As would be appreciated by one of ordinary skill in the art sequences with substantial sequence similarity may be homologs of one another.

Substantial sequence identity: As used herein, the phrase “substantial sequence identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutic regimen: A “therapeutic regimen,” as that term is used herein, refers to a dosing regimen whose administration across a relevant population may be correlated with a desired or beneficial therapeutic outcome.

Therapeutically effective amount: As used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element included of a plurality of amino acids having designated positions relative to one another in linear or three-dimensional space and/or contributing to a particular structural motif and/or biological function; a nucleic acid may have a characteristic sequence element included of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid includes about 10, about 9, about 8, about 7, about 6, about 5, about 4, about 3, about 2, or about 1 substituted residues as compared to a reference. Often, a variant polypeptide or nucleic acid includes a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid includes not more than about 5, about 4, about 3, about 2, or about 1 addition or deletion, and, in some embodiments, includes no additions or deletions, as compared to the reference. In some embodiments, a variant polypeptide or nucleic acid includes fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### BRIEF DESCRIPTION OF THE DRAWING

FIG. 1 is a table showing information related to selected S/MARs identified as SUM1-6 (see Table 4). The first column identifies the S/MAR and its length. The second, third, and fourth columns respectively indicate the level of expression of a gene naturally operably linked with the indicated S/MAR in liver, muscle, and CNS. The fifth column shows S/MAR binding proteins (S/MARbps) for which binding sites are found in each indicated S/MAR, and the expression level of each S/MARbp in liver, muscle, and CNS, respectively.

FIG. 2 is a chart showing relative light units resulting from expression of a luciferase coding sequence operably linked with the indicated S/MAR in HEK 293T cells.

FIG. 3 is a chart showing relative light units resulting from expression of a luciferase coding sequence operably linked with the indicated S/MAR in Neuro2A cells.

FIG. 4 is a set of images including panels A-D. Each of panels A-D is an image showing expression of a GFP coding sequence operably linked with the indicated S/MAR.

FIG. 5 is a schematic of well imaging together with a chart showing the number of GFP expressing cells over time for transfected 293T cell lines, each respectively expressing a GFP coding sequence operably linked with one of the four indicated S/MARs.

FIG. 6 is a set of images including two rows of panels. Panels of the top row represent cells in which a GFP coding sequence is operably linked with SUMhuIFNb. Panels of the bottom row represent cells in which a GFP coding sequence is operably linked with SUM1. In each column of panels, images represent a particular time point.

FIG. 7 is a chart showing relative light units (RLU) resulting from expression of a luciferase coding sequence operably linked with the indicated S/MAR in C2C12 mouse myoblasts.

FIG. 8 is a set of three charts showing relative light units (RLU) produced by expression of luciferase in mouse tissues following hydrodynamic intravenous injection of plasmids encoding luciferase linked with indicated S/MARs (SUM1, SUM2 or SUM3, respectively) or a linked with a reference S/MAR (SUMhu1FNb). Charts represent liver, kidney, and heart tissues, respectively.

### DETAILED DESCRIPTION

The present disclosure provides methods and compositions in which S/MARs are operably linked with one or more coding sequences to achieve a particular, improved, or target level, duration, and/or tissue specificity of coding sequence expression. Thus, the present disclosure provides a gene therapy expression platform that includes a nucleic acid sequence that is an S/MAR, where the S/MAR is positioned such that it is or can be operably linked with a coding sequence of interest. The present disclosure further includes that S/MARs provided herein can control expression in one or more tissues or cell types selected from liver, retina, skeletal muscle, and neurons or the central nervous system. The present disclosure includes the recognition that such nucleic acids can be delivered to systems or subjects using various viral and non-viral delivery vehicles, including without limitation a mini-nucleosome (e.g., a modified mini-nucleosome).

One particular problem solved by the present disclosure is the need for nucleic acid sequences that can be used in gene therapy to achieve a particular, improved, or target level, duration, and/or tissue specificity of expression of a coding sequence, such as a coding sequence that encodes a therapeutic protein. Another particular problem solved by the present disclosure is the need to increase the stability of exogenous nucleic acid sequences (such as plasmid DNA) delivered to target cells and/or nuclei thereof, e.g., for gene therapy. In various embodiments, these problems are particularly relevant to tissues or cell types effected by various conditions, such as liver, retina, skeletal muscle, and neurons or central nervous system, where S/MARs provided herein are particularly useful to achieving improved, or target level, duration, and/or tissue specificity of expression of a coding sequence, such as a coding sequence that encodes a therapeutic protein, in one or more of these tissues or cell types. In various embodiments, improvement in a level, duration, and/or tissue specificity of expression can be determined by comparison to a reference such as a reference construct that does not include the S/MAR or a reference construct that includes a control S/MAR such as hu1FNb.

Expression level and expression specificity are factors that contribute to the success of gene therapy. Certain vectors in current use have been found to provide insufficient expression and/or are accompanied by toxicities, e.g., as in the case of retroviral vectors found to cause oncogenesis in clinical trials or adeno-associated viruses (AAVs) associated with immunogenicity. Various other vectors are limited by payload capacity, e.g., as in the case of AAVs. Non-viral vectors have historically been characterized by low transfection efficiency and low duration of expression (see, e.g., Koirala et al., 2013). Expression vectors of the present disclosure are characterized by enduring expression and low toxicity, and therefore provide a solution to these challenges.

Expression vectors including S/MARs operably linked with coding sequences are demonstrated to provide enduring expression of coding sequences when maintained episomally, and further are mitotically stable during cell division. Without wishing to be bound by any particular scientific theory, certain of these advantages can arise at least in part from the ability of S/MARs to protect expression of encoded transgenes from repression (barrier activity) and increase expression from promoters (stimulatory activity). Vectors including huIFNb have been shown to provide duration of expression of at least 6 months.

To provide one particular example, plasmid DNA expression vectors can be used, for example, in many forms of non-viral gene transfer. However, it has been observed in many animal models that expression of expression products encoded by plasmid DNA vectors is of short duration, e.g., in that a greater duration of expression would improve therapeutic or other target outcomes. This can be a significant challenge in the use of plasmid DNA vectors, e.g., for gene therapy. Exogenous DNA such as plasmid DNA that does not integrate into a host cell genome is lost from progeny cells during one or more rounds of cell division. When exogenous DNA such as plasmid DNA remains in a cell, e.g., a differentiated cell, in an episomal or extrachromosomal state, the exogenous DNA can become silenced, e.g., by transgene silencing. The present disclosure includes that use of an S/MAR as disclosed herein in an exogenous sequence reduces loss of exogenous DNA and/or silencing of exogenous DNA. Without wishing to be bound by any particular scientific theory, this is at least in part because an S/MAR can anchor chromatin to the nuclear matrix proteins during interphase, thereby promoting episomal retention in the host cell nucleus. Binding of exogenous DNA with nuclear matrix can produce partitioned looped domains that contribute structurally to the tight packaging of DNA in the nucleus, and functionally to the regulation of gene expression and replication of the genome.

The present disclosure includes, in various embodiments, expression vectors that include an S/MAR that confers (e.g., causes or contributes to a level of expression of an operably linked coding sequence that is comparable to and/or greater than the level of expression from a reference expression vector that includes the reference huIFNb S/MAR (e.g., under controlled, same, and/or comparable conditions). The present disclosure includes, in various embodiments, expression vectors that include an S/MAR that confers a duration of expression of an operably linked coding sequence that is comparable to and/or greater than the duration of expression from a reference expression vector that includes the reference huIFNb S/MAR. The present disclosure includes, in various embodiments, expression vectors that include an S/MAR that confer tissue specificity to expression of an operably linked coding sequence. In various embodiments, an S/MAR of the present disclosure is an S/MAR of Table 3 or having at least 70% identity thereto (preferably at least 80% identity thereto, and still more preferably at least 90% or 95% identity thereto). In various embodiments, an S/MAR of the present disclosure is an S/MAR according to one of SEQ ID NOs: 1-10 as set forth in Table 4, or having at least 70% identity thereto (preferably at least 80% identity thereto, and still more preferably at least 90% or 95% identity thereto).

Methods and compositions of the present disclosure are advantageous for expression of coding sequences. In various embodiments, expression is in particular tissues. In various embodiments, expression vectors of the present disclosure are particularly useful, e.g., for gene therapy and/or in gene therapy in particular tissues.

### Mammalian Scaffold Matrix Attachment Regions (S/MARs)

Putative S/MARs have been identified by bioinformatics approaches but the use of S/MARs in expression vectors and the expression characteristics they confer to operably linked sequences have not been fully explored. Without wishing to be bound by any particular scientific theory, S/MARs participate in the organization of DNA in the nuclei of cells around the nuclear matrix, which organization of DNA contributes to the selective accessibility of DNA for expression. Anchoring of nucleic acids to nuclear matrix plays a structural role in maintaining higher order chromatin conformation and a functional role in regulating DNA replication and gene expression. S/MARs promote association of nucleic acid sequences with euchromatin and transcription factors. S/MARs can increase the stability and expression of nucleic acid sequences.

The present disclosure includes the recognition that presence of an S/MAR in an expression vector can achieve a particular, improved, or target level, duration, and/or tissue specificity of expression of an operably linked heterologous coding sequence, e.g., when the expression vector is introduced into host cell nuclei as exogenous DNA (e.g., where improvement in a level, duration, and/or tissue specificity of expression can be determined by comparison to a reference such as a reference construct that does not include the S/MAR or a reference construct that includes a control S/MAR such as hu1FNb). S/MARs can also enhance replication of exogenous DNA, including episomal DNA.

Narwade (2019 Nucleic Acids Research 47(14): 7247-7261) identified 298,443 binding sites of proteins thought to interact with S/MARs (S/MAR binding proteins, or S/MARbps) including BRCA1, BRIGHT, SMAR1, CEBPB, CUX1/CDP, CTCF, Fast1/FOXH1, HoxC11, Ku autoantigen, NMP4, Mut-p53, SAF-A/hnRNPU, SATB1, and YY1. Narwade found that 283,568 of these were further characterized by at least one further S/MAR feature selected from presence of (i) an origin of replication (OriC) sequence, (ii) AT richness, (iii) kinked DNA, (iv) curved DNA, (v) TG richness, (vi) MAR signature, and (vii) a Topoisomerase-II site (of which 52,567 were characterized by several of these seven features, and 190 were characterized by each of these seven features). In various embodiments, such S/MAR features are identified by the following characteristic sequences: (i) presence of an OriC sequence selected from ATTA, ATTTA, and ATTTTA; (ii) presence of an AT rich sequence according to WWWWWW, where each W is independently selected from A o r T, or by the presence of two such motifs optionally separated by 8, 9, 10, 11, or 12 intervening nucleotides; (iii) presence of a kinked DNA sequence selected from TAN₃TGN₃CA, TAN₃CAN₃TG, TGN₃TAN₃CA, TGN₃CAN₃TA, CAN₃TAN₃TG, or CAN₃TGN₃TA, where each N is independently selected from any nucleotide; (iv) presence of a curved DNA sequence selected from AAAAN₇AAAAN₇AAAA, TTTTN₇TTTTN₇TTTT, or TTTAAA, where each N is independently selected from any nucleotide; (v) presence of a TG rich sequence selected from TGTTTTG, TGTTTTTTG, or TTTTGGGG; (vi) presence of a MAR signature sequence made up of an AATAAYAA sequence and an AWWRTAANNWWGNNNC where each W is independently selected from A or T, each Y is an independently selected pyrimidine, each R is an independently selected purine, and each N is independently selected from any nucleotide, and where the two sequences (AATAAYAA and AWWRTAANNWWGNNNC) are less than 200 bp apart; and (vii) presence of a Topoisomerase-II site selected from RnYnnCnnGYnGKTnYnY or GTnWAYATTnATnnR, where each R is independently selected from A or G, each Y is independently selected from T or C, each W is independently selected from A or T, and each K is independently selected from G or T.

Narwade concluded that bioinformatics identified a total of 283,568 S/MARs. The 283,568 S/MARs have lengths ranging from 33 bp to 61,755 bp, and cumulatively represent 230,177.6 kb, or 7.5% of the human genome. Thus, bioinformatic efforts to identify S/MARs have provided insights regarding the potential number of S/MARs, their genomic distribution, and positioning relative to genes or coding sequences. However, the predictive power of those approaches for identification of functional S/MARs has not been globally established and use of various S/MARs or sets of SMARs in expression vectors have not been explored.

S/MARs that have been experimentally validated include EpMAR in the immunoglobulin heavy chain locus, the apoB MAR from the human apolipoprotein B locus, the Ch-LysMAR from the chicken lysozyme locus, and the huIFNb MAR from the human IFNblocus. Thus, with few exceptions, use of S/MARs in expression vectors to achieve a particular, improved, or target level, duration, and/or tissue specificity of expression of an operably linked heterologous coding sequence, such as a coding sequence that encodes a therapeutic protein, has not been explored.

The present disclosure includes identification of S/MARs particularly useful to achieve a particular, improved, or target level, duration, and/or tissue specificity of expression of a coding sequence, such as a coding sequence that encodes a therapeutic protein, in one or more tissues or cell types selected from liver, retina, skeletal muscle, and neurons or the central nervous system. The present disclosure includes identification of S/MARs that include S/MARbp binding sites of S/MARbps preferentially expressed in liver, retina, skeletal muscle, and neurons or the central nervous system.

The present disclosure includes expression vectors that include an S/MAR selected from Table 3, which provides 190 S/MARs identified as including all of (i) an origin of replication (OriC) sequence, (ii) AT richness, (iii) kinked DNA, (iv) curved DNA, (v) TG richness, (vi) MAR signature, and (vii) a Topoisomerase-II site. Thus, in various embodiments, an S/MAR of the present disclosure is an S/MAR selected from Table 3. In various embodiments, the present disclosure includes expression vectors that include an S/MAR having at least 70% sequence identity with an S/MAR provided in Table 3 (e.g., at least 70, 75, 80, 85, 90, 95, 96, 97, 98, 99, or 100% identity with an S/MAR provided in Table 3; preferably at least 80% identity with an S/MAR provided in Table 3, and still more preferably at least 90% or 95% identity with an S/MAR provided in Table 3). S/MAR positions and sequences correspond to human genome assembly GhCR38/hg38. In various embodiments, S/MAR positions can correspond to human genome assembly hg19.

In various embodiments, an S/MAR has at least 70% sequence identity with an S/MAR provided in Table 3 (e.g., at least 70, 75, 80, 85, 90, 95, 96, 97, 98, 99, or 100% identity with an S/MAR provided in Table 3; preferably at least 80% identity with an S/MAR provided in Table 3, and still more preferably at least 90% or 95% identity with an S/MAR provided in Table 3) and includes 1, 2, 3, 4, 5, 6, or all of (i) an origin of replication (OriC) sequence, (ii) AT richness, (iii) kinked DNA, (iv) curved DNA, (v) TG richness, (vi) MAR signature, and (vii) a Topoisomerase-II site. In various embodiments, an S/MAR has at least 70% sequence identity with an S/MAR provided in Table 3 (e.g., at least 70, 75, 80, 85, 90, 95, 96, 97, 98, 99, or 100% identity with an S/MAR provided in Table 3; preferably at least 80% identity with an S/MAR provided in Table 3, and still more preferably at least 90% or 95% identity with an S/MAR provided in Table 3) and includes one or more (e.g., 1 or more, 2 or more, 3 or more, 4 or more, 5 or more, 6 or more, 7 or more, 8 or more, 9 or more, 10 or more, 11 or more, 12 or more, 13 or more, or 14), or all, of the S/MARbp binding sites indicated in Table 3 for the particular S/MAR.

In various embodiments, an S/MAR has at least 70% sequence identity with an S/MAR provided in Table 3 (e.g., at least 70, 75, 80, 85, 90, 95, 96, 97, 98, 99, or 100% identity with an S/MAR provided in Table 3; preferably at least 80% identity with an S/MAR provided in Table 3, and still more preferably at least 90% or 95% identity with an S/MAR provided in Table 3), where the S/MAR (a) includes 1, 2, 3, 4, 5, 6, or all of (i) an origin of replication (OriC) sequence, (ii) AT richness, (iii) kinked DNA, (iv) curved DNA, (v) TG richness, (vi) MAR signature, and (vii) a Topoisomerase-II site, and (b) includes one or more (e.g., 1 or more, 2 or more, 3 or more, 4 or more, 5 or more, 6 or more, 7 or more, 8 or more, 9 or more, 10 or more, 11 or more, 12 or more, 13 or more, or 14), or all, of the S/MARbp binding sites indicated in Table 3 for the particular S/MAR.

In various embodiments, the present disclosure includes expression vectors that include an S/MAR selected from Table 4 (SEQ ID NOs: 1-10). In various embodiments, the present disclosure includes expression vectors that include an S/MAR having at least 70% sequence identity with an S/MAR provided in Table 4 (e.g., at least 70, 75, 80, 85, 90, 95, 96, 97, 98, 99, or 100% identity with an S/MAR provided in Table 4; preferably at least 80% identity with an S/MAR provided in Table 4, and still more preferably at least 90% or 95% identity with an S/MAR provided in Table 4).

In various embodiments, an S/MAR has at least 70% sequence identity with an S/MAR provided in Table 4 (e.g., at least 70, 75, 80, 85, 90, 95, 96, 97, 98, 99, or 100% identity with an S/MAR provided in Table 4; preferably at least 80% identity with an S/MAR provided in Table 4, and still more preferably at least 90% or 95% identity with an S/MAR provided in Table 4) and includes 1, 2, 3, 4, 5, 6, or all of (i) an origin of replication (OriC) sequence, (ii) AT richness, (iii) kinked DNA, (iv) curved DNA, (v) TG richness, (vi) MAR signature, and (vii) a Topoisomerase-II site. In various embodiments, an S/MAR has at least 70% sequence identity with an S/MAR provided in Table 4 (e.g., at least 70, 75, 80, 85, 90, 95, 96, 97, 98, 99, or 100% identity with an S/MAR provided in Table 4; preferably at least 80% identity with an S/MAR provided in Table 4, and still more preferably at least 90% or 95% identity with an S/MAR provided in Table 4) and includes one or more (e.g., 1 or more, 2 or more, 3 or more, 4 or more, 5 or more, 6 or more, 7 or more, 8 or more, 9 or more, 10 or more, 11 or more, 12 or more, 13 or more, or 14), or all, of the S/MARbp binding sites indicated in Table 4 for the particular S/MAR.

In various embodiments, an S/MAR has at least 70% sequence identity with an S/MAR provided in Table 4 (e.g., at least 70, 75, 80, 85, 90, 95, 96, 97, 98, 99, or 100% identity with an S/MAR provided in Table 4; preferably at least 80% identity with an S/MAR provided in Table 4, and still more preferably at least 90% or 95% identity with an S/MAR provided in Table 4), where the S/MAR (a) includes 1, 2, 3, 4, 5, 6, or all of (i) an origin of replication (OriC) sequence, (ii) AT richness, (iii) kinked DNA, (iv) curved DNA, (v) TG richness, (vi) MAR signature, and (vii) a Topoisomerase-II site, and (b) includes one or more (e.g., 1 or more, 2 or more, 3 or more, 4 or more, 5 or more, 6 or more, 7 or more, 8 or more, 9 or more, 10 or more, 11 or more, 12 or more, 13 or more, or 14), or all, of the S/MARbp binding sites indicated in Table 4 for the particular S/MAR.

In various embodiments, the present disclosure includes an S/MAR synthesized to include 1, 2, 3, 4, 5, 6, or all of (i) an origin of replication (OriC) sequence, (ii) AT richness, (iii) kinked DNA, (iv) curved DNA, (v) TG richness, (vi) MAR signature, and (vii) a Topoisomerase-II site. In various embodiments, the present disclosure includes an S/MAR synthesized to include each of (i) an origin of replication (OriC) sequence, (ii) AT richness, (iii) kinked DNA, (iv) curved DNA, (v) TG richness, (vi) MAR signature, and (vii) a Topoisomerase-II site.

In various embodiments, the present disclosure includes an S/MAR synthesized to include (a) 1, 2, 3, 4, 5, 6, or all of (i) an origin of replication (OriC) sequence, (ii) AT richness, (iii) kinked DNA, (iv) curved DNA, (v) TG richness, (vi) MAR signature, and (vii) a Topoisomerase-II site, and (b) one or more (e.g., 1 or more, 2 or more, 3 or more, 4 or more, 5 or more, 6 or more, 7 or more, 8 or more, 9 or more, 10 or more, 11 or more, 12 or more, 13 or more, or 14), or all, of the S/MARbp binding sites found in Table 4. In various embodiments, the present disclosure includes an S/MAR synthesized to include (a) 1, 2, 3, 4, 5, 6, or all of (i) an origin of replication (OriC) sequence, (ii) AT richness, (iii) kinked DNA, (iv) curved DNA, (v) TG richness, (vi) MAR signature, and (vii) a Topoisomerase-II site, and (b) one or more (e.g., 1 or more, 2 or more, 3 or more, 4 or more, 5 or more, 6 or more, 7 or more, 8 or more, 9 or more, or 10), or all, of the S/MARbp binding sites BRIGHT, CEBPB, CTCF, FOXH1, Ku autoantigen, NMP4, Mut p53, hnRNPU, SATB1, YY1. In various embodiments, the present disclosure includes an S/MAR synthesized to include (a) each of (i) an origin of replication (OriC) sequence, (ii) AT richness, (iii) kinked DNA, (iv) curved DNA, (v) TG richness, (vi) MAR signature, and (vii) a Topoisomerase-II site and (b) one or more (e.g., 1 or more, 2 or more, 3 or more, 4 or more, 5 or more, 6 or more, 7 or more, 8 or more, 9 or more, 10 or more, 11 or more, 12 or more, 13 or more, or 14), or all, of the S/MARbp binding sites found in Table 4. In various embodiments, the present disclosure includes an S/MAR synthesized to include (a) each of (i) an origin of replication (OriC) sequence, (ii) AT richness, (iii) kinked DNA, (iv) curved DNA, (v) TG richness, (vi) MAR signature, and (vii) a Topoisomerase-II site and (b) one or more (e.g., 1 or more, 2 or more, 3 or more, 4 or more, 5 or more, 6 or more, 7 or more, 8 or more, 9 or more, or 10), or all, of the S/MARbp binding sites BRIGHT, CEBPB, CTCF, FOXH1, Ku autoantigen, NMP4, Mut p53, hnRNPU, SATB1, YY1. Binding sites of S/MARbps are known in the art and certain such S/MARbp binding sites are additionally provided herein. In various embodiments, a BRIGHT S/MARbp binding site can have a sequence according to AACTTGTTTATT (see, e.g., Identification of a matrix-associated region 5′ of an immunoglobulin heavy chain variable region gene. C F Webb, C Das, K L Eneff, and P W Tucker. Mol Cel Biol, 1991) or another binding site thereof known in the art, or a sequence having no more than 1, 2, or 3 differences from any such binding site. In various embodiments, a CEBPB S/MARbp binding site can have a sequence according to RTTGCGYAAY (where R is A or G, and each Y is independently selected from C or T) (see, e.g., DNA binding specificity of the CCAAT/enhancer-binding protein transcription factor family. J Biol Chem, 1996) or another binding site thereof known in the art, or a sequence having no more than 1, 2, or 3 differences from any such binding site. In various embodiments, a CTCF S/MARbp binding site can have a sequence according to the consensus sequence CCGCGNGGNGGCAG (see, e.g., Kim T H, Abdullaev Z K, Smith A D, Ching K A, Loukinov D I, Green R D, Zhang M Q, Lobanenkov V V, Ren B (March 2007). “Analysis of the vertebrate insulator protein CTCF-binding sites in the human genome”. Cell. 128 (6): 1231-45. doi:10.1016/j.cell.2006.12.048. PMC 2572726. PMID 17382889) or another binding site thereof known in the art, or a sequence having no more than 1, 2, or 3 differences from any such binding site. In various embodiments, a FOXH1 S/MARbp binding site can have a sequence according to AATHMCA where M is selected from A or C and H is selected from A or T or C (see, e.g., Foxhl Occupies cis-Regulatory Modules Prior to Dynamic Transcription Factor Interactions Controlling the Mesendoderm Gene Program. Rebekah M. Charney, Elmira Forouzmand, Jin Sun Cho, Jessica Cheung, Kitt D. Paraiso, Yuuri Yasuoka, Shuji Takahashi, Masanori Taira, Ira L. Blitz, Xiaohui Xie, Ken W. Y. Cho. Dev Cell. 2017) or another binding site thereof known in the art, or a sequence having no more than 1, 2, or 3 differences from any such binding site. In various embodiments, a Ku autoantigen S/MARbp binding site can have a sequence according to the octamer motif (ATGCAAA), which is considered to be a direct binding site for Ku without involving translocation from the DNA end (see, e.g., Sequence-specific DNA Binding and Transcription Factor Phosphorylation by Ku Autoantigen/DNA-dependent Protein Kinase: PHOSPHORYLATION OF Ser-527 OF THE RAT GLUCOCORTICOID RECEPTOR. WardGiffin, JoannaKwast-Welfeld, David J. Rodda, Gratien G. Prefontaine, MayaTraykova-Andonova, YixianZhang, Nancy L. Weigel, Yvonne A. Lefebvre, Robert J. G. Hache. JBC, 1997) or another binding site thereof known in the art, or a sequence having no more than 1, 2, or 3 differences from any such binding site. In various embodiments, an NMP4 S/MARbp binding site can have a sequence according to TTCTTTTTTTTTCTTTTTCT and/or TTATTTTTTTTTCTTTGCCT (see, e.g., DNA Binding and Gene Activation Properties of the Nmp4 Nuclear Matrix Transcription Factors. Kitti Torrungruang, MartaAlvarez, RitaShah, Jude E. Onyia, Simon J. Rhodes, Joseph P. Bidwell. JBC, 2002) or another binding site thereof known in the art, or a sequence having no more than 1, 2, or 3 differences from any such binding site. In various embodiments, a YY1 S/MARbp binding site can have a sequence according to CGCCATNTT and/or GCCGCCATTTTG (see, e.g., YY1's longer DNA-binding motifs. Jeong do Kim, Joomyeong Kim. Genomics, 2009) or another binding site thereof known in the art, or a sequence having no more than 1, 2, or 3 differences from any such binding site. In various embodiments, an hnRNPU S/MARbp binding site can have a sequence according to UUAGGGAG, NNAGGGAG, UUAGGNAG, or NNAGGNAG, or can bind an MII human SAR element from the DNA topoisomerase I gen. (see, e.g., Nucleic-acid-binding properties of hnRNP-U/SAF-A, a nuclear-matrix protein which binds DNA and RNA in vivo and in vitro. Frank O. Fackelmayer, Kirsten Dahm, Andrea Renz, Uwe Ramsperger and Arndt Richter. JBC, 1994 and/or The scaffold/matrix attachment region binding protein hnRNP-U (SAF-A) is directly bound to chromosomal DNA in vivo: a chemical cross-linking study. Biochemistry, 1997) or another binding site thereof known in the art, or a sequence having no more than 1, 2, or 3 differences from any such binding site. In various embodiments, a Mutp53 S/MARbp binding site can have a sequence according to TGGGGAGGGTGGGGAGGGTGGGGAAGG (see, e.g., c-MYC promoter G-quadruplex formed at the 50-end of NHE 1111 element: insights into biological relevance and parallel-stranded G-quadruplex stability. Raveendra I. Mathad, Emmanuel Hatzakis, Jixun Dai and Danzhou Yang. NAR, 2011) or another binding site thereof known in the art, or a sequence having no more than 1, 2, or 3 differences from any such binding site. In various embodiments, an STAB1 S/MARbp binding site can have a sequence according to CGAAAGGAATTGGAATAAAAATTTC (see, e.g., A novel SATB1 binding site in the BCL2 promoter region possesses transcriptional regulatory function. Feiran Gong, Luan Sun, and Yujie Sun. J Biomed Res, 2010) or another binding site thereof known in the art, or a sequence having no more than 1, 2, or 3 differences from any such binding site. Notwithstanding various S/MARbp binding sites known in the art and/or the disclosure of S/MARbp binding sites provided herein, those of skill in the art will further appreciate that binding sites can be identified by known techniques of molecular biology (see, e.g., use of ChIP-Seq, as just one example, in Narwade (2019 Nucleic Acids Research 47(14): 7247-7261)).

In various embodiments, an S/MAR of the present disclosure causes expression in one or more of liver, skeletal muscle, and neurons or the central nervous system. In various embodiments, expression in one or more of liver, skeletal muscle, and neurons or the central nervous system is tissue specific.

In certain exemplary embodiments, SUM1 (SEQ ID NO: 1) causes expression (e.g., tissue specific expression) in liver, optionally wherein a particular, improved, or target level or duration of expression is achieved in liver.

In certain exemplary embodiments, SUM2 (SEQ ID NO: 2) causes expression (e.g., tissue specific expression) in muscle, optionally wherein a particular, improved, or target level or duration of expression is achieved in muscle.

In certain exemplary embodiments, SUM3 (SEQ ID NO: 3) causes expression (e.g., tissue specific expression) in one or more of liver, skeletal muscle, and neurons or the central nervous system, optionally wherein a particular, improved, or target level or duration of expression is achieved in liver, skeletal muscle, and neurons or the central nervous system.

In certain exemplary embodiments, SUM4 (SEQ ID NO: 4) causes expression (e.g., tissue specific expression) in one or more of liver, skeletal muscle, and neurons or the central nervous system, optionally wherein a particular, improved, or target level or duration of expression is achieved in liver, skeletal muscle, and neurons or the central nervous system.

In certain exemplary embodiments, SUM5 (SEQ ID NO: 5) causes expression (e.g., tissue specific expression) in one or more of liver, skeletal muscle, and neurons or the central nervous system, optionally wherein a particular, improved, or target level or duration of expression is achieved in liver.

In certain exemplary embodiments, SUM6 (SEQ ID NO: 6) causes expression (e.g., tissue specific expression) in one or more of liver, skeletal muscle, and neurons or the central nervous system, optionally wherein a particular, improved, or target level or duration of expression is achieved in liver, skeletal muscle, and neurons or the central nervous system.

In some embodiments, SUM1 causes expression (e.g., tissue specific expression) in one or more, or all, of tissues or cell types selected from liver, retina, skeletal muscle, and neurons or the central nervous system, as supported by the Examples of the present specification.

In various such embodiments, improvement in a level, duration, and/or tissue specificity of expression can be determined by comparison to a reference such as a reference construct that does not include the S/MAR or a reference construct that includes a control S/MAR such as hu1FNb.

TABLE 3

| Chromo- | S/MAR | | S/MAR | Distance | | |
|---|---|---|---|---|---|---|
| some | Start | S/MAR Stop | Length | To TSS | S/MAR Location | ChIP Binding* |
| chr11 | 59555548 | 59558668 | 3121 | 36490 | Distal Intergenic | (2), (4), (9), (10), (14) |
| chr3 | 85494834 | 85495318 | 485 | 109124 | Intron | (4) |
| | | | | | (uc003dqk.4/253559, | |
| | | | | | intron 1 of 9) | |
| chr21 | 8201881 | 8261075 | 59195 | 0 | Promoter (<=1 kb) | (1), (2), (4), (6), (7), (8), (9), |
| | | | | | | (10), (12), (3), (14) |
| chr21 | 8384823 | 8412462 | 27640 | 0 | Promoter (<=1 kb) | (1), (2), (4), (6), (7), (8), (9), |
| | | | | | | (10), (12), (3), (14) |
| chr21 | 8412470 | 8450701 | 38232 | 0 | Promoter (<=1 kb) | (1), (4), (7), (8), (12), (3), (14) |
| chr7 | 148939262 | 148942757 | 3496 | −20558 | Distal Intergenic | (2), (4), (6), (9), (14) |
| chrX | 39625704 | 39626256 | 553 | −34960 | Distal Intergenic | (4) |
| chr3 | 114745246 | 114747215 | 1970 | −1703 | Promoter (1-2 kb) | (4), (14) |
| chr15 | 101957228 | 101967775 | 10548 | 0 | Promoter (<=1 kb) | (1), (2), (4), (6), (9), (12), (14) |
| chr2 | 113579836 | 113586367 | 6532 | 0 | Promoter (<=1 kb) | (1), (2), (4), (9), (12), (3), (14) |
| chr22_{—} | 118014 | 179767 | 61754 | 0 | Promoter (<=1 kb) | (1), (2), (4), (6), (7), (8), (9), |
| KI270733v1 | | | | | | (10), (12), (13), (3), (14) |
| random | | | | | | |
| chrUn_{—} | 101319 | 161834 | 60516 | 0 | Promoter (<=1 kb) | (1), (2), (4), (6), (7), (8), (9), |
| GL000220v1 | | | | | | (10), (12), (3), (14) |
| chr21 | 8986504 | 9000569 | 14066 | 0 | Promoter (<=1 kb) | (1), (4), (6), (9), (10), (12), (14) |
| chr2 | 158502808 | 158504562 | 1755 | −28623 | Intron | (2), (4) |
| | | | | | (uc061oum.1/8502, | |
| | | | | | intron 1 of 21) | |
| chr17 | 82036507 | 82039071 | 2565 | 0 | Promoter (<=1 kb) | (4), (6), (9), (12), (14) |
| chr6 | 26320945 | 26324697 | 3753 | −35436 | Distal Intergenic | (2), (4), (6), (9), (10), (14) |
| chr6 | 26248991 | 26252202 | 3212 | 0 | Promoter (<=1 kb) | (2), (4), (6), (9), (14) |
| chr6 | 27751867 | 27754607 | 2741 | 53324 | Distal Intergenic | (2), (4), (6), (9), (10), (12), (14) |
| chr6 | 27806377 | 27811459 | 5083 | 0 | Promoter (<=1 kb) | (1), (2), (4), (6), (9), (12), (14) |
| chr6 | 26120515 | 26126947 | 6433 | 0 | Promoter (<=1 kb) | (1), (2), (4), (9), (12), (14) |
| chr3 | 9900854 | 9903726 | 2873 | 0 | Promoter (<=1 kb) | (9), (14) |
| chr12 | 124909219 | 124918771 | 9553 | 0 | Promoter (<=1 kb) | (1), (2), (4), (6), (9), |
| | | | | | | (10), (12), (14) |
| chr1 | 159589198 | 159592619 | 3422 | 1373 | Promoter (1-2 kb) | (14) |
| chr3 | 157359288 | 157360677 | 1390 | 3804 | Intron | (4), (14) |
| | | | | | (uc062pik.1/uc062pik.1, | |
| | | | | | intron 2 of 2) | |
| chr6 | 9412329 | 9413472 | 1144 | 628151 | Distal Intergenic | (4), (6), (14) |
| chr17 | 16397613 | 16399210 | 1598 | 16148 | Distal Intergenic | (2), (4), (14) |
| chr14 | 35383582 | 35386463 | 2882 | 16775 | Distal Intergenic | (6), (9), (14) |
| chr4 | 176377467 | 176378335 | 869 | 50511 | Distal Intergenic | (6) |
| chr6 | 134173473 | 134177083 | 3611 | 0 | Promoter (<=1 kb) | (1), (2), (4), (6), (9), |
| | | | | | | (10), (12), (14) |
| chr6 | 26551756 | 26557501 | 5746 | 13318 | Distal Intergenic | (2), (4), (9), (10), (14) |
| chr6 | 27833988 | 27840433 | 6446 | 0 | Promoter (<=1 kb) | (1), (2), (4), (9), (12), (14) |
| chr20 | 17856008 | 17861419 | 5412 | 82203 | Distal Intergenic | (9), (10), (14) |
| chr11 | 1671547 | 1675370 | 3824 | 5950 | Distal Intergenic | (14) |
| chr6 | 34243675 | 34247333 | 3659 | 1652 | Promoter (1-2 kb) | (4), (6), (9), (12), (14) |
| chr4 | 49308956 | 49319009 | 10054 | 280264 | Distal Intergenic | (1), (2), (4), (6), (9), (12), (14) |
| chr4 | 49319011 | 49331693 | 12683 | 290319 | Distal Intergenic | (4), (6), (9), (12), (13), (3), (14) |
| chr4 | 49509930 | 49516265 | 6336 | 481238 | Distal Intergenic | (1), (4), (6), (9), (12), (13), (14) |
| chr16 | 8567282 | 8571590 | 4309 | 712 | Promoter (<=1 kb) | (1), (2), (4), (6), (9), (10), (14) |
| chr6 | 137904161 | 137906995 | 2835 | 28323 | Distal Intergenic | (2), (4), (10) |
| chr1 | 225885989 | 225892298 | 6310 | 0 | Promoter (<=1 kb) | (4), (7), (9), (14) |
| chr20 | 54154892 | 54156328 | 1437 | 970 | Promoter (<=1 kb) | (4) |
| chr14 | 43130377 | 43132667 | 2291 | 1374612 | Distal Intergenic | (12) |
| chr14 | 43218730 | 43221957 | 3228 | 1285322 | Distal Intergenic | (12) |
| chr2 | 58201561 | 58203774 | 2214 | −35758 | Intron | (12) |
| | | | | | (uc010fce.4/55120, | |
| | | | | | intron 6 of 12) | |
| chr14 | 37593885 | 37596761 | 2877 | 0 | Promoter (<=1 kb) | (6), (7), (9), (14) |
| chr15 | 74441215 | 74444937 | 3723 | 4374 | Downstream (1-2 kb) | (7), (9), (14) |
| chr14 | 68783481 | 68797753 | 14273 | 0 | Promoter (<=1 kb) | (1), (2), (4), (6), (7), (9), (10), |
| | | | | | | (12), (14) |
| chr2 | 85323742 | 85329865 | 6124 | 0 | Promoter (<=1 kb) | (1), (2), (4), (7), (9), |
| | | | | | | (12), (3), (14) |
| chr17 | 38671180 | 38675853 | 4674 | 0 | Promoter (<=1 kb) | (2), (4), (6), (9), (12), (14) |
| chr5 | 138463213 | 138471586 | 8374 | 0 | Promoter (<=1 kb) | (1), (2), (4), (6), (9), |
| | | | | | | (10), (12), (14) |
| chr5 | 177929153 | 177931187 | 2035 | 65055 | Distal Intergenic | (14) |
| chr4 | 112273375 | 112274563 | 1189 | 7210 | 3′ UTR | (10) |
| chr2 | 119290697 | 119292641 | 1945 | −41626 | Distal Intergenic | (6), (9), (14) |
| chr12 | 63801805 | 63802631 | 827 | −1074 | Promoter (1-2 kb) | (6) |
| chr7 | 149623076 | 149626210 | 3135 | −88571 | Distal Intergenic | (1), (2), (4), (6), (9), |
| | | | | | | (10), (12), (14) |
| chr11 | 82873775 | 82874415 | 641 | −15004 | Intron | (14) |
| | | | | | (uc001ozs.4/5547, | |
| | | | | | intron 1 of 8) | |
| chr3 | 9411978 | 9412892 | 915 | −10319 | Intron | (10) |
| | | | | | (uc062gjv.1/55209, | |
| | | | | | intron 1 of 9) | |
| chr8 | 26704439 | 26706754 | 2316 | 66909 | Distal Intergenic | (14) |
| chr16 | 89559612 | 89566812 | 7201 | 0 | Promoter (<=1 kb) | (1), (4), (6), (9), (10), (12), (14) |
| chr3 | 187263122 | 187266163 | 3042 | 0 | Promoter (<=1 kb) | (4), (14) |
| chr3 | 47281229 | 47284226 | 2998 | 0 | Promoter (<=1 kb) | (2), (4), (6), (9), (14) |
| chr8 | 127034735 | 127036725 | 1991 | 21581 | Distal Intergenic | (4), (6), (9), (14) |
| chr3 | 100634592 | 100636052 | 1461 | 0 | Promoter (<=1 kb) | (6), (9), (14) |
| chr1 | 110336205 | 110342038 | 5834 | 0 | Promoter (<=1 kb) | (1), (4), (6), (7), (9), |
| | | | | | | (10), (3), (14) |
| chr11 | 65496354 | 65509910 | 1355 | 0 | Promoter (<=1 kb) | (1), (2), (4), (6), (9), |
| | | | | | | (10), (12), (14) |
| chr12 | 10721572 | 10723830 | 2259 | 0 | Promoter (<=1 kb) | (4), (9), (10), (14) |
| chr9 | 131108470 | 131117753 | 9284 | 0 | Promoter (<=1 kb) | (4), (9), (14) |
| chr2 | 11387444 | 11390875 | 3432 | −3106 | Exon | (4), (9), (14) |
| | | | | | (uc061gsc.1/uc061gsc.1, | |
| | | | | | exon 1 of 3) | |
| chr13 | 112744521 | 112745120 | 600 | 9573 | Intron | (4) |
| | | | | | (uc001vsi.4/23250, | |
| | | | | | intron 1 of 29) | |
| chr14 | 23634689 | 23648131 | 13443 | 0 | Promoter (<=1 kb) | (4), (9), (14) |
| chr21 | 31657610 | 31661434 | 3825 | 0 | Promoter (<=1 kb) | (4), (9), (14) |
| chr16 | 66829948 | 66831606 | 1659 | 0 | Promoter (<=1 kb) | (1), (6), (9), (12), (14) |
| chr5 | 91103209 | 91104129 | 921 | 208366 | Intron | (4), (14) |
| | | | | | (uc003kju.4/84059, | |
| | | | | | intron 87 of 89) | |
| chr22 | 45316836 | 45319412 | 2577 | 0 | Promoter (<=1 kb) | (4), (6), (10) |
| chr6 | 159690550 | 159694774 | 4225 | 0 | Promoter (<=1 kb) | (1), (2), (4), (6), (9), (12), (14) |
| chr10 | 25492999 | 25494353 | 1355 | 26809 | Intron | (4), (7), (14) |
| | | | | | (uc001isj.4/57512, | |
| | | | | | intron 5 of 10) | |
| chr4 | 99350247 | 99352004 | 1758 | 726 | Promoter (<=1 kb) | (14) |
| chr12 | 21772592 | 21777089 | 4498 | 0 | Promoter (<=1 kb) | (6), (14) |
| chr9 | 72361808 | 72367138 | 5331 | 0 | Promoter (<=1 kb) | (1), (4), (6), (9), (14) |
| chr20 | 400001 | 403263 | 3263 | −4235 | Distal Intergenic | (2), (4), (9), (12), (14) |
| chr22 | 23240360 | 23249252 | 8893 | −1780 | Promoter (1-2 kb) | (4), (6), (9), (12), (14) |
| chr7 | 123635383 | 123636303 | 921 | −10426 | Intron | (6) |
| | | | | | (uc003vkv.2/142685, | |
| | | | | | intron 13 of 13) | |
| chr1 | 233654457 | 233657078 | 2622 | 0 | Promoter (<=1 kb) | (4), (14) |
| chr5 | 54387283 | 54388416 | 1134 | −2422 | Promoter (2-3 kb) | (4) |
| chr20 | 58888055 | 58894267 | 6213 | 0 | Promoter (<=1 kb) | (1), (4), (6), (7), (9), (10), (14) |
| chr2 | 96207094 | 96209996 | 2903 | 0 | Promoter (<=1 kb) | (1), (2), (4), (6), (9), (12), (14) |
| chr1 | 78002614 | 78007257 | 4644 | 0 | Promoter (<=1 kb) | (2), (4), (12), (14) |
| chr1 | 179925823 | 179927166 | 1344 | 21857 | Exon | (4) |
| | | | | | (uc057nqy.1/uc057nqy.1, | |
| | | | | | exon 2 of 3) | |
| chr16 | 8739551 | 8742526 | 2976 | −3476 | Intron | (9), (14) |
| | | | | | (uc002czc.5/18, | |
| | | | | | intron 2 of 15) | |
| chr3 | 52773269 | 52779924 | 6656 | 0 | Promoter (<=1 kb) | (9), (14) |
| chr2 | 200587727 | 200588845 | 1119 | 0 | Promoter (<=1 kb) | (14) |
| chr1 | 245100755 | 245101568 | 814 | −5611 | Intron | (4) |
| | | | | | (uc001ibd.3/84288, | |
| | | | | | intron 7 of 7) | |
| chr17 | 54967503 | 54971777 | 4275 | 0 | Promoter (<=1 kb) | (1), (2), (4), (6), (9), |
| | | | | | | (10), (3), (14) |
| chr2 | 237599691 | 237603292 | 3602 | −8598 | Exon | (6), (9), (14) |
| | | | | | (uc061uay.1/uc061uay.1, | |
| | | | | | exon 1 of 2) | |
| chr17 | 16214033 | 16217933 | 3901 | 0 | Promoter (<=1 kb) | (4), (6), (9), (10), (12), (14) |
| chr2 | 101251108 | 101254024 | 2917 | 0 | Promoter (<=1 kb) | (2), (4), (6), (9), (10), (12), (14) |
| chr19 | 13115794 | 13120146 | 4353 | 0 | Promoter (<=1 kb) | (2), (4), (9), (14) |
| chr16 | 73172386 | 73173287 | 902 | −27939 | Distal Intergenic | (6), (14) |
| chr10 | 30604307 | 30607007 | 2701 | 22755 | Distal Intergenic | (4), (9), (14) |
| chr7 | 135145702 | 135151998 | 6297 | 0 | Promoter (<=1 kb) | (2), (4), (5), (9), (14) |
| chr10 | 46022266 | 46023993 | 1728 | 0 | Promoter (<=1 kb) | (4), (6), (9), (12), (14) |
| chr6 | 106084296 | 106087025 | 2730 | 0 | Promoter (<=1 kb) | (4), (6), (7), (9), (10), (14) |
| chr12 | 79688709 | 79693819 | 5111 | 0 | Promoter (<=1 kb) | (4), (6), (9), (14) |
| chr11 | 65415424 | 65433798 | 18375 | 0 | Promoter (<=1 kb) | (1), (2), (4), (6), (5), (9), (10), |
| | | | | | | (12), (14) |
| chr3 | 122382141 | 122385197 | 3057 | 0 | Promoter (<=1 kb) | (4), (6), (9), (14) |
| chr4 | 83495303 | 83496932 | 1630 | −9748 | Intron | (4), (14) |
| | | | | | (uc062xwm.1/84142, | |
| | | | | | intron 1 of 6) | |
| chr17 | 76351248 | 76355478 | 4231 | 0 | Promoter (<=1 kb) | (2), (6), (7), (9), (14) |
| chr2 | 42791383 | 42797251 | 5869 | 0 | Promoter (<=1 kb) | (6), (9), (10), (12), (14) |
| chr1 | 91497496 | 91504360 | 6865 | 0 | Promoter (<=1 kb) | (2), (4), (6), (9), (12), (14) |
| chr20 | 53736594 | 53742012 | 5419 | 128240 | Distal Intergenic | (2), (4), (8), (9), (14) |
| chr17 | 18181812 | 18185833 | 4022 | 0 | Promoter (<=1 kb) | (1), (2), (4), (6), (9), (14) |
| chr10 | 14542896 | 14544006 | 1111 | 4634 | Intron | (6), (14) |
| | | | | | (uc001imx.3/83641, | |
| | | | | | intron 1 of 3) | |
| chr20 | 58649408 | 58653452 | 4045 | 0 | Promoter (<=1 kb) | (1), (2), (4), (6), (9), |
| | | | | | | (10), (12), (14) |
| chr2 | 159795791 | 159799167 | 3377 | 0 | Promoter (<=1 kb) | (4), (9), (14) |
| chr7 | 41188324 | 41189313 | 990 | 86720 | Distal Intergenic | (4) |
| chr9 | 130933735 | 130943775 | 10041 | 0 | Promoter (<=1 kb) | (4), (6), (9), (14) |
| chr21 | 36909202 | 36911364 | 2163 | 19053 | Intron | (4) |
| | | | | | (uc021wjb.1/3141, | |
| | | | | | intron 6 of 11) | |
| chr14 | 61761712 | 61764031 | 2320 | 0 | Promoter (<=1 kb) | (9), (10), (14) |
| chr1 | 112617793 | 112623116 | 5324 | 0 | Promoter (<=1 kb) | (2), (4), (9), (10), (14) |
| chr19 | 38974468 | 38977104 | 2637 | 0 | Promoter (<=1 kb) | (2), (6), (9), (14) |
| chr2 | 68318383 | 68321072 | 2690 | 0 | Promoter (<=1 kb) | (4), (14) |
| chr5 | 134523768 | 134528704 | 4937 | 0 | Promoter (<=1 kb) | (1), (4), (6), (7), (9), |
| | | | | | | (10), (3), (14) |
| chr10 | 58334554 | 58336164 | 1611 | 0 | Promoter (<=1 kb) | (6), (9), (14) |
| chr5 | 96714033 | 96715576 | 1544 | 11176 | Exon | (2), (4), (14) |
| | | | | | (uc063foy.1/831, | |
| | | | | | exon 2 of 2) | |
| chr12 | 108559614 | 108564359 | 4746 | 0 | Promoter (<=1 kb) | (1), (2), (4), (6), (9), (14) |
| chr16 | 66205586 | 66206101 | 516 | −160521 | Distal Intergenic | (4) |
| chrX | 66900797 | 66901555 | 759 | −261499 | Distal Intergenic | (14) |
| chr6 | 4017544 | 4019509 | 1966 | −1758 | Promoter (1-2 kb) | (2), (4), (6), (9), (14) |
| chr4 | 98927447 | 98930632 | 3186 | 0 | Promoter (<=1 kb) | (2), (4), (6), (5), (9), (10), (14) |
| chr2 | 63838948 | 63843589 | 4642 | 0 | Promoter (<=1 kb) | (1), (2), (4), (6), (9), (10), (14) |
| chr2 | 64157456 | 64159420 | 1965 | −13002 | Intron | (4), (14) |
| | | | | | (uc061jtc.1/100507006, | |
| | | | | | intron 1 of 5) | |
| chr11 | 102449674 | 102454271 | 4598 | 0 | Promoter (<=1 kb) | (4), (6), (7), (9), (14) |
| chr10 | 94679904 | 94685514 | 5611 | 0 | Promoter (<=1 kb) | (4), (12), (14) |
| chr10 | 46396148 | 46399783 | 3636 | 0 | Promoter (<=1 kb) | (4), (6), (7), (9), (14) |
| chr1 | 99764193 | 99767589 | 3397 | 0 | Promoter (<=1 kb) | (4), (9), (14) |
| chr2 | 38690536 | 38691472 | 937 | 9241 | Intron | (2), (4), (14) |
| | | | | | (uc002rqy.4/130589, | |
| | | | | | intron 4 of 6) | |
| chr12 | 19186389 | 19187971 | 1583 | −17291 | Intron | (4), (6), (14) |
| | | | | | (uc001rdz.5/54477, | |
| | | | | | intron 3 of 3) | |
| chr3 | 141388593 | 141390263 | 1671 | 781 | Promoter (<=1 kb) | (4), (14) |
| chr10 | 26486325 | 26487958 | 1634 | 47900 | Intron | (4), (14) |
| | | | | | (uc001iss.4/54518, | |
| | | | | | intron 2 of 14) | |
| chr3 | 165782697 | 165784837 | 2141 | 52583 | Intron | (14) |
| | | | | | (uc003fem.5/590, | |
| | | | | | intron 3 of 3) | |
| chr15 | 89127834 | 89130806 | 2973 | 11330 | Intron | (4), (6), (9), (14) |
| | | | | | (uc059myz.1/11057, | |
| | | | | | intron 3 of 4) | |
| chr19 | 44304416 | 44305921 | 1506 | 0 | Promoter (<=1 kb) | (4), (9), (14) |
| chr7 | 94654956 | 94659159 | 4204 | 0 | Promoter (<=1 kb) | (6), (9), (14) |
| chr2 | 8138261 | 8142283 | 4023 | −19937 | Exon | (4), (9), (14) |
| | | | | | (uc061gle.1/uc061gle.1, | |
| | | | | | exon 1 of 2) | |
| chr1 | 40152572 | 40154223 | 1652 | −7150 | Distal Intergenic | (4), (6), (10) |
| chr10 | 25078157 | 25079885 | 1729 | −15878 | Distal Intergenic | (4), (9), (12), (14) |
| chr12 | 102923630 | 102925486 | 1857 | −6027 | Intron | (14) |
| | | | | | (uc058sk1.1/5053, | |
| | | | | | intron 1 of 4) | |
| chr16 | 52544523 | 52549338 | 4816 | 0 | Promoter (<=1 kb) | (6), (9), (14) |
| chr6 | 7137169 | 7144115 | 6947 | 0 | Promoter (<=1 kb) | (2), (6), (9), (12), (14) |
| chr4 | 80727925 | 80728380 | 456 | −141669 | Intron | (4) |
| | | | | | (uc003hmf.3/255119, | |
| | | | | | intron 3 of 5) | |
| chr20 | 41027047 | 41030323 | 3277 | 0 | Promoter (<=1 kb) | (4), (9), (12), (14) |
| chr2 | 65434524 | 65438917 | 4394 | −1887 | Promoter (1-2 kb) | (1), (2), (4), (6), (7), (9), (14) |
| chr22 | 21859373 | 21869846 | 10474 | 0 | Promoter (<=1 kb) | (2), (4), (6), (7), (9), (14) |
| chr12 | 21756286 | 21758544 | 2259 | 0 | Promoter (<=1 kb) | (14) |
| chr8 | 86289498 | 86291213 | 1716 | 29933 | Intron | (4), (9), (14) |
| | | | | | (uc064oeo.1/157724, | |
| | | | | | intron 1 of 3) | |
| chr9 | 109117518 | 109121121 | 3604 | 0 | Promoter (<=1 kb) | (1), (2), (4), (6), (9), (3), (14) |
| chr5 | 126369494 | 126372057 | 2564 | 0 | Promoter (<=1 kb) | (4), (9), (12), (14) |
| chr12 | 60844747 | 60846394 | 1648 | 980839 | Distal Intergenic | (14) |
| chr14 | 58429847 | 58430602 | 756 | 1857 | Promoter (1-2 kb) | (4) |
| chr16 | 53089090 | 53092339 | 3250 | −6821 | Intron | (4), (9), (14) |
| | | | | | (uc059uec.1/80205, | |
| | | | | | intron 1 of 38) | |
| chr4 | 147730902 | 147733294 | 2393 | 0 | Promoter (<=1 kb) | (9), (14) |
| chr11 | 30990531 | 30993121 | 2591 | 0 | Promoter (<=1 kb) | (4), (14) |
| chr3 | 182682063 | 182684144 | 2082 | −109356 | Distal Intergenic | (6), (9), (14) |
| chr2 | 68282614 | 68283450 | 837 | −21384 | Downstream (<1 kb) | (6), (14) |
| chr2 | 111908334 | 111909603 | 1270 | 9670 | Intron | (9), (14) |
| | | | | | (uc002thk.2/10461, | |
| | | | | | intron 1 of 18) | |
| chrX | 41083817 | 41088421 | 4605 | 0 | Promoter (<=1 kb) | (1), (2), (4), (9), (10), (14) |
| chr7 | 18638935 | 18639554 | 620 | 129622 | Intron | (4) |
| | | | | | (uc011jya.3/9734, | |
| | | | | | intron 9 of 12) | |
| chr3 | 152306951 | 152308533 | 1583 | 6753 | Intron | (4), (12), (11) |
| | | | | | (uc062pck.1/4154, | |
| | | | | | intron 2 of 9) | |
| chr15 | 36645017 | 36645551 | 535 | 4412 | 5′ UTR | (6) |
| chr9 | 89137668 | 89139813 | 2146 | 38954 | Intron | (4), (14) |
| | | | | | (uc004aqf.2/53358, | |
| | | | | | intron 1 of 11) | |
| chr2 | 200318663 | 200322592 | 3930 | 9713 | Intron | (4), (9), (12) |
| | | | | | (uc061rdv.1/26010, | |
| | | | | | intron 1 of 4) | |
| chr2 | 142869344 | 142871154 | 1811 | −6344 | Distal Intergenic | (4), (10), (12), (14) |
| chr13 | 51642813 | 51645740 | 2928 | 58305 | Intron | (4), (14) |
| | | | | | (uc001vfp.4/115825, | |
| | | | | | intron 1 of 11) | |
| chr12 | 81036778 | 81037237 | 460 | −39974 | Intron | (4) |
| | | | | | (uc058rjb.1/79611, | |
| | | | | | intron 2 of 3) | |
| chr10 | 121925916 | 121929784 | 3869 | 0 | Promoter (<=1 kb) | (1), (4), (6), (9), (10), (14), (11) |
| chr12 | 99699265 | 99699738 | 474 | 6886 | Intron | (4) |
| | | | | | (uc001tge.3/56899, | |
| | | | | | intron 8 of 25) | |
| chr3 | 105365346 | 105370356 | 5011 | 0 | Promoter (<=1 kb) | (1), (4), (6), (7), (9), (10), (14) |
| chr6 | 130364974 | 130367614 | 2641 | 0 | Promoter (<=1 kb) | (4), (6), (9), (14) |
| chr15 | 76668469 | 76669310 | 842 | −2811 | Promoter (2-3 kb) | (4), (7) |
| chr12 | 50548521 | 50551318 | 2798 | 43414 | Intron | (4), (9), (14) |
| | | | | | (uc001rwu.4/57609, | |
| | | | | | intron 1 of 7) | |
| chr12 | 11648409 | 11651501 | 3093 | 0 | Promoter (<=1 kb) | (2), (4), (6), (9), (10), (14) |
| chr14 | 34331425 | 34333478 | 2054 | −108548 | Intron | (4), (6), (9), (14) |
| | | | | | (uc059ard.1/uc059ard.1, | |
| | | | | | intron 1 of 4) | |
| chr10 | 50621680 | 50626385 | 4706 | 0 | Promoter (<=1 kb) | (2), (4), (6), (9), (10), (12), (14) |
| chr6 | 16758600 | 16763569 | 4970 | 0 | Promoter (<=1 kb) | (4), (6), (7), (9), (10), (12), (14) |
| chr3 | 67875054 | 67876894 | 1841 | −127322 | Intron | (4), (14) |
| | | | | | (uc003dnc.4/101927111, | |
| | | | | | intron 5 of 6) | |
| chr2 | 72673082 | 72673974 | 893 | 152067 | Intron | (12) |
| | | | | | (uc010fep.4/23233, | |
| | | | | | intron 6 of 21) | |
| chr17_{—} | 55374 | 58117 | 2744 | NA | NA | (6), (9) |
| GL000205v2_{—} | | | | | | |
| random | | | | | | |
| chr1_{—} | 163680 | 166627 | 2948 | NA | NA | (1), (6), (9), (12) |
| KI270706v1_{—} | | | | | | |
| random | | | | | | |
| chrUn_{—} | 150539 | 159011 | 8473 | NA | NA | (4), (6), (9), (12), (13), (14) |
| KI270743v1 | | | | | | |
| chrUn_{—} | 159166 | 164552 | 5387 | NA | NA | (6), (8), (9), (12), (13), (14) |
| KI270743v1 | | | | | | |

*Binding proteins are coded as follows: (1) BRCA1, (2) BRIGHT, (3) SMAR1, (4) CEBPB, (5) CUX1/CDP, (6) CTCF, (7) Fast1/FOXH1, (8) HoxC11, (9) Ku autoantigen, (10) NMP4, (11) Mut-p53, (12) SAF-A/hnRNPU, (13) SATB1, and (14) YY1

TABLE 4

| | SEQ ID | |
|---|---|---|
| S/MAR | NO. | Sequence |
| SUM1 | 1 | AGAATGTACCTTATTTTGAGCAATGTTTGTACATATTTAAAAGTAATTTCTATTGTT |
| | | ATTTCTGTGAACTGTCTACACCTAAGATGTCTAAATTCTGATTATTTCTCTAATGGG |
| | | ATATTTTGTACCTGTCTTGGGGATTTCCCAGAGGTTTTTCAACATGGTCTCACAGTC |
| | | CAGATAATACCACCTGACTTAAAGAGGTCCATCCCACAATCAATTGTCTTTTGGGG |
| | | TTATCTCACCGTGCAGCCTATTTTAAGAAAAACATACCTTTGTCCTGCCAATGGTG |
| | | GAGTCCAGCTTCTTCTCTGGACTTCTTTTCTGTCTATGCTCAATATTCAAAGAAGTT |
| | | ATCCAGTTTCTTGCCTTTATACACGAGTTAGGCAGTTTCTACACCTGCAAAATCCA |
| | | GAGGACGTAGGCCAAGCTCTCTGGTAGGTTCTTTTTAACCACCCTAACTTGGTATA |
| | | AGGTAAAGAGGAGGGAATACTTCTCCCCTTCCAATGGAAGCAAAAGAGAAATGTC |
| | | ACAGCACATTGACACATCTCTAGTGAAATTCTCTTCCCAGCCATTTGTTTTAGTCTT |
| | | CTGGTTTGTTCTTATAAATCCTTACAGTGAGTATTGAATCTAATGCTGCAAAATGG |
| | | GATTCTCCATCTCTTAGCATGCATGTCCTGTATAGAAAGTCTCAAACTCCAAAGCA |
| | | GCAAAAACAAAACAATCACATTCAATGTACTATTAGCAGCAACTAGACTGATGGG |
| | | TTTTTTTTTTAAATTAAGCTACTTCCTTAAATATTTAACCAGTTTTATGCATTTGTAA |
| | | AGAATTTAACAAATGGAGATGTTAGAGACAATTTCTATGGCATTTCTATATATCTT |
| | | GGACTACCTTTTCAAGGATATATGCACAGCAAAGAGCATTAGAGGCTAGTCTGTCC |
| | | CTTTGGGTCAGACAGCAGAATTATTTATTGTCCAGTATAATAATTACAGAGAACAT |
| | | CTCCATCTCTGAAGACATTCCATAATAATAAAGTCTTCAACCTCTCACCAGAAGAA |
| | | AACTTGTTTCCATGCCAAGGTAAAGATAATGCCTATCTTTGAAGAGGAGGATGAG |
| | | GAAGTTGCCATCAGCTTCTGTATGAATCAGAATGTCCTAAGCTCAGTGTTCCTTGG |
| | | CTGTGAAACTGTATGCTCAGCATACACCTGGATCTACTCTGCATTACCCATGGACT |
| | | TGGGGGACAAGGAGAATCAATGAAAATATGGACCTCATGTTGCCTATTGTGCCCT |
| | | GAACAATAAACTGTGTAAATTCATTTGGATTCCTGGTTTACTCATTTGCCAATGTTA |
| | | TGAAAGTGTGGAAAGTCTGCCTAGAAGTTACACTGATGTTCAGGAATTGCTTGGCA |
| | | AGTTGATAGGAAAGAAAGGGACCTGTGGCATATACAGCTAGGTTCTCCATTTTCTT |
| | | TCCTTCTTTCGCATGGAAATGGAAACTCAATATTAGTTTTCCCCAATCCTTCATAGA |
| | | ATAAAAACTGCCTTTCCCAGCCTTTCTTGCAGCTGAGTGTAACTACAGGTCTAATA |
| | | TTTGGCCAAAGAAATGCAAGGAGAAGTAGTCTGTGCAATACCTAAGTAGTGTCCA |
| | | TAGAGGAGGCACACCCTTCTCCTTTTATTTTCTCCTTCTTGCAGGATGGAATGCAGA |
| | | TATGCTGACTGGAGCAGGAGCAGCCATATTGCATTATAAGGTAAATTTAGGAATG |
| | | GAGACATGTTTTTCAGAGCAAAGGAGGAAAGGTTCAAGACAGAAGAAGCATTTGT |
| | | GGTTATTGATATCATAAAGCATCACTTCAGCAGCCTTAGACTGCTGACTTAGAATT |
| | | TGATGAGAAAAATTAACTTCTACTTTCTTTAAACTCATTTTTTTGACGTTTCTGTCA |
| | | CTCACA |
| SUM2 | 2 | GGGGGTCTGATATGAGTTTGTGCATGTGTGCAGGGTGAGTGAGACAATCCATATAT |
| | | TTGAGAAAAGACAGTGAAATCTTTAAAGGTAAAAATAATAAAACCAGCTGTGGCA |
| | | CCTATTGGATAAGGAATTACTAACTGATGATTTAGAAATTCCTTTATGTGCGTTAG |
| | | GAGCTCATTTGTTATCCCCATTTTACAGATGCGCAGCCCCAGGTTCAGCAATTTGCT |
| | | CTAAATGAGACTGTAGAGGCTAATTCCAGCCCCCGGCAGCTCTCACCTTAAGCTGC |
| | | CTGGCTGAGGTCAGCAGTAAGTTACCTGGCAGCGTCCGGGGGCCTGCAGCCGAGG |
| | | ACAGGGCTGGAACTACACTTCCCAGTGTGCCAGGCGGGGCGGGGCGCGGCGGGGA |
| | | GGGCGCAGGCTCCTTCCCAGAAGCAAAAGCGGAAACAAAAGAGTCTCGCCGGCGT |
| | | CCCCGCCCGCACACTCGCGCACACTCGCGCTCGGGCGCACACGGAGCAGGGACCG |
| | | GCGCCCGGAGCGAGCCAGGGAGCGGCTAACCGGGGACCCACCGCGCGGAGCCAG |
| | | CCTAGCTGCCAGCGAGCCCAACCCGCGACGACCCACGCCCCTGAGCCCCGCAGCC |
| | | GACCCCTGCCGGCCGGTGTCCCCACCGCCATCCCTGACCCATGGCGCTGAAGAGG |
| | | ATTCAGAAAGTGAGTGCCGGGGCCGGGCCTGGGGCTGCGGGGCAGCGGCCCCCTG |
| | | CCCACGCTGACTCGGCCAGTGGTCCCGAGAGACATGCGGGGAGAGCAAGGGATGG |
| | | AAGGGTGGGCCGGGGTGCGGGCAGGGAGCTGAAGGCTCGGGCCGGGCCAGCGGG |
| | | CATCGGACAGGTGAGGTGGGAGCAGGGTCAGGTCCCAGGCGGCCGGAGGAGCTTG |
| | | AAGGCTGGGGGCCCGCGCGCGTTCGCGCCGGAGCCGCTCCCGGAGCCCCAGGGTA |
| | | GAGAGCCTCTTGCGCGTCCAGACCGTCCCGAAATCTGTGCTTCACGGTCACGGCCG |
| | | GGTTTTGCCCTCCATGGGGCTAGTTTTGACCCCTGTCATGCTGTGTTTTCTCCAGGC |
| | | TGTTTCTTAAGCCTGTCCTCTTTTATGTTCTTGCTTACCCGGCCTCCCCGGGAGGGC |
| | | AAGGAAGGTGGGGTGGGCCTGCAGCCTTTCTCCGCGGAGCTGTCGGGTTGGTTCTT |
| | | TCTCTCCTCTGCGGTTGGCTCTTGCTCAGTTGCCCGAATGGGCTGGAAAGCCCATTT |
| | | GCGGCCAAGAAGTCAGGAGAAGGCTCCTGCCCCTTCTATTTTGGTGCCTCTGCTGC |
| | | TGCCACTTCAGGTCACATAGCTGTAGCGAAAACATAGTTGCTGATACACAACAAG |
| | | ATGACCTCCTGGCTGTGATTCACGGTGAGTCAAGCCAAACAGGTTTTTTTCCCCCT |
| | | CATGATAAACTTAGATGTAAAGTTCGAGACACAACATAAACTGTCGCAAATAAAT |
| | | TGTTTTGATGCTTTGACACATGGGTATAGAATGAGGAAGAAATATATATCTTCATC |
| | | CAACAACTGTTTACCCATCTCAACCCTGTGTATGGAATCGTCTTTTTTTCACAGTAT |
| | | TTATTGCCACCATTTCAGATGCGAACATCCTTCAGAATTCAGATTCGGGCATTTCT |
| SUM3 | 3 | TTAGCTGTGTTTTCTATTCCTCTGCTTGAGAGCTCTCTTTGTCTTTTTTTGAGTTTTA |
| | | CTTTATTTTTAATTTACAAATAATAATTGTATATATTCATGAGGTACAATGTGATGG |
| | | TTTGGATACTTGGATACATGTTTACATCACGGAATGATCAAATCAGGCCAATTAGC |
| | | TTAACCATCACCTCAAATATTTATTTCTTTGTGGTGAAAACGTTTAAAATCTTCTCT |
| | | TTTAGTGTTTTGAAATATGCAATACATTATTATTAACTGTCACACAGTATTGTGCAA |
| | | TAGATCACCAGAACTTATTCCTCATGTCTAACTGAACTGTGTACCCATTGACTAAC |
| | | ATCTCCCCTTTCTCCAGCCCCCTCCCCAGCCTCTGGTAACCACCATTCTACTCTCTG |
| | | CTTCTATTAATTTGACTTTTTTTTTTTCAGACAGGGTCTCACTCTGTCACCCAGGCT |
| | | GGAGTGTAGTGGCATGATCTCGGCTCACTGCAGCCTCCATCTCCTTCGCTCAAATG |
| | | ATCCTCCCACCTCAGCCTCCCAAGTAGCTGGGACTATAGGCAT |
| SUM4 | 4 | AAGAGTTGAGACCCTGCCTCAAAAAAAAAACAAAAAAACACAAAACTATCACATT |
| | | TTGTATTATCATATGCAAATTGATCTGCCTACAGCCAAAGCTTACCATTAATCTAC |
| | | AGCTATGCCCCATCATTCAAACTTGTTCAAAAGATTGTATTATAGAAATTTTACTTC |
| | | TAGATAGAATGTGGATTAAATCTCATTGTAGATTTTGTATAAAATGATGAATTTCT |
| | | GATACCACATACCTTTGTTCAGGCTTTGTTTGTCTCTTGTTTTTGTTGTGTTGTTTTT |
| | | AACAGCTTCATCACTGGTCCAGCTGTAATACCAGGGTACTTCTCCGTTGATGTGAA |
| | | TAATGTGGTACTCATTTTAAATGGAAGAGAAAAAGCAAAGATCTTTTATGCCACCC |
| | | AGTGGTTACTTTATGCACAAAATTTAGTGCAAATTCAAAAACTCCAGCATCTTGCT |
| | | GTTGTTTTGCTCGGAAATGAACATTGTGATAATGAGTGGATAAACCCATTCCTCAA |
| | | AAGAAATGGAGGCTTCGTGGAGCTGCTTTTCATAATATATGACAGCCCCTGGATTA |
| | | ATGACGTGGATGTTTTTCAGTGGCCTTTAGGAGTAGCAACGTAAGTACAAAATATG |
| | | ATTAAACATTTTTAGGCCCTAACACCTGAATTTCTGAACCAGGAAGACTTATTCAA |
| | | AATTGTAATAAATTTAGTTCTTCTGGGATTATTGAGGCCAGGTACAGTCTTTCTCAG |
| | | AGATAAAAACAGGGTGTGGTCGCATCCTTCAATAACAATTATGATGTTTTTAAGAG |
| | | TATTTTATTCTTTAATGAGGAGATGAGTTACTTTTTTTCTT |
| SUM5 | 5 | CATTTTTTTCTTCTTTTGCATAATAGAATGGTATAGTAAACACAACACTGGCTTTGT |
| | | TGTTTAGACAAACAAGTCTTCAATTCCAATTCTAATTCTGATTGAGAAAACCTAAC |
| | | CTCTCAGAGGCCCAGTTTCTCTGTTAAAAGGGGATCATACCACTTACTTCATAAAG |
| | | AGTGATCATGAGCATTCAATCCACTTATTTGTTAAGGGCCTACCGTATGCCAGTCA |
| | | GTGTTCCAGGCTTATGAGATACAGCAGAGCACAGAAATAACAAGTCCTTGCGTGC |
| | | ATGGAGCTTACATTGTGCTGGGGAGATAGCAAGCATATAAATACGTGGAAATGTG |
| | | TAATGTCAGGAAGTGAGAAGTGCTAATATAAAAAATAAAGCACAGCGTGGGGACA |
| | | GAGAGTATGCAGGATACTTAGAGGGGATGGATGTTTTGGCCTGTCTGAAAGGGTG |
| | | GCATTTGAGCAGTGACTGGGATGCAGTGAGGAGCAAGCCATGCAAACATCTGGGG |
| | | AGACAGCATCCAGGCAGTGGGAATGGCAGGTGCAAAGGTCCTGAGGTAGGAGGA |
| | | TTTCTGTGTGCTTAAGGAGGTAGGTTGGTGGGCCTGGCAACATGGGCAGTGGCCTG |
| | | ATTACTAGGGCTTTATAGGTGATGGTAAGGGTTTGGGTTAAATGAAGAAACAGGG |
| | | ATTGTGAGTCTAACATCTCCTCGCTTCTCCTTCCCCCACTTCCAAACAAATATTGGT |
| | | ATTTTTAACATAATGGCCAACAAGTTTGTGCTCTATTGATTTTTAAAATTAGATAAG |
| | | CTAGTTCTTATCTACGGACTCACTTTGTCTATGGGCCAAATGTCCTGTGGTTATTTG |
| | | TAAAGTATAATCTGGAGATATTGATTCTGTTTTTGCAATTACCCATTAGAAGAAAT |
| | | TGTGTTAATCTTTCCCTTTCCTTCTCCTCCCTTCTAAATTCTGTTAGGTGTGTGCTTC |
| | | GGTACTTAATAAACAAGCCTGATTACTTACATGGAAAGAACTAGAATAAGCTTTTT |
| | | TTTTTTTTTTTTTTTTGAGATCGAGTCTCACTCTGTTGCCTCCTGAGTAGCTGGGACT |
| | | ATGAAACTGCAAGCACCCACCACCACGCTAATTTTTGTATTTTTAGTAGAGATGGG |
| SUM6 | 6 | CTCTGCCTCCCAGGTTCAAGCGATTCTCCTGCCTCAGCCTCCTGAGTAGCTGGGATT |
| | | ACAGGCATGCGCCACCACACCCGGCTGATTTTGTATTTTTAGTAGAGACAGGGTTT |
| | | CTCCATGTTGGCCAAGCTGGCCTCGAACTCCTGACCTCAGGTGATCCACCCGCCTC |
| | | GGCCTCCCAAAGTGCTGGGATTATAAGCGTGAGCCATGGCACCTGGCCAACTCTTT |
| | | CAACTAATTGCCAATCAGAAAAACTTTAAAATCTGCCTGTGACCTGGATACACTCC |
| | | TCACACGCACTTCGAGTTGTCCTGCCCTTCCATATGGAACCAACGTAAGTCCTATA |
| | | TGTATTTGACTGATGTCTCATGTCTCCCTAAAATGTATAAAACTAGGCTGTGTCTGA |
| | | GCACCTTGGGCACATGTTCTCAGGATCTCCTGAGGGCTGTGTCACAGGCCATTGAT |
| | | CACTCATATTTGGCTCAGAATAAATCTCCTCAAATACTTTTTTGTCAACAGTCCACT |
| | | GGGCTCAGGTGAGCAGGGCCTGAAACAGATGACATGGAAATTTATAATAAAATAA |
| | | GGGGCCCGTGCCTTAAGACATACCACAAAGTAAATATAACACTGTACAGGCTCTCT |
| | | CGGGTGACCCTGGACTACAGAATGTGGGTTGTGGGGAGGGAGAGACGTGGACTTT |
| | | TTCCTGCAGGTCATGGCTTGGGTGCACCTAGAAACAGGCCAATACCACCTCTTCTT |
| | | TTCTGGAAAGGTGGGGCCTGACAGCTTTGTGTGGGGAACCAGTGTCCTGTTACAGT |
| | | TCAGGTCTAAAATGCCTGCTGAGTAAATAAACATGTTGTTCTCAACCTAAACCGAA |
| | | AAGCCTGATATTTGTAGGAATAAAATTGATCTTTCCTACCCTGAGCAAAGTGCTCC |
| | | AAATATGCTCAGTCATGGAGTGGAGACAGATAAGTAAACAGACGCTGGGAATAGA |
| | | GTGGAGACAGATAAATAAACAGATGCTGGGGATATGTCGTGATACAGAGCCAGGC |
| | | CAGAGTCAAGCAAGGTGCTGCATTGGCATTCTGCCGGCCTTTCCTGCAGGAAAAG |
| | | GTGTGAAAAAGCTCTTGGGGTGATGGGAAGGGGTGTGCTGTTTTGAAATCGGAAG |
| | | ATAAAAACAGAGCCATGGAGGTTCTGCGCCATCGCCTTGGAACAGATGGTATCTG |
| | | CCTCCACATCTGCAATGACTCAGATCTCTGCTCTGGGCATACCTGCAGTCAAATCA |
| | | GGGCTGCCCAGACATCCTGTGTTTGTGAAATGCTGAGAAAAACAGAAGGGGACTA |
| | | ACTGGCTTCTAGCAGTTGCCTACTCATCCACCCTCTTCCAAACACCCAGGCTGGGG |
| | | AGTAACATTTTGGGGATGAGTCCTCGTAATGCTGCTTCTCTCTAGAGAGCAATGTG |
| | | TGAAAATTGACCCAAGGAGTTGTCTCATGGGGTGAGGCATGATTGGCTGTCTTTTC |
| | | TCAGAGCCTCCCTGAGGCTCATCTACCCCCATGCCCTGAGCTCACTGGGGCCTGCC |
| | | TGGCCAAACAGAATATACCCATTAAGGGAAATAAATTTCCAGGCCACAATAACAA |
| | | GCATCACCACTTTGAAAAGAAAGAACCATAAACTGAA |
| SUM7 | 7 | CAGATGGGCAGGGTAGGGGTTGGGTGGGAACAGAAGTTGGAGAGTGGATGTGGCT |
| | | GCCAACCACACAAGTTGTGCCAACCCACAGATTGAGGAAAGATGCTAAATTTGGA |
| | | ATCTGGCAAACCAGTGTTTGGTTCTTAGCTCTGCCACTTCTAAGCTGTGTGAAACTT |
| | | GGTTGAGGTCCCTAACTTCTCCTGAGGGTGAACAACTCACAAAGTTGTTTTGCTTA |
| | | TTAAATGTGATAACACCTGTAAACATCTAACAGAGTGCCTAGCACATAGCAGGGA |
| | | TCTAGCAATTGAATTAGGGTTATTTGTTTCTGTCTACTGATTGGGTATTGTTTCTGA |
| | | CACTTACCCAAGTGTGAATAGCCTATAACACTGGTATAATTTGTGAAATGATGCTG |
| | | CCATCTAGTGAAAACCAAGACACACACACACACACACACACACACACATACACAC |
| | | ACACGTGCGCGCGCATGGACACCCAGCTTCACCAATGACAATATGGATTGGCATG |
| | | TTTTAGCCTCACAACACAGAGCCCTGGGGCTAACTGGCACCTAGAGAGGTCATCTC |
| | | GGCCAGTGCCTTCCAAACTACCAGTGCTGAAAAGCCAGTTCAAAAAATTTTGAACC |
| | | CATTGCACACCAATATTTTTGTGAAATAATAAAAATAAATTACTGGAAAAATGAA |
| | | ATAAAAAATATGTATAAAATACAAACCAAAATTTTAGAACTGTTAGATTCAACAG |
| | | CAAAAAATTGCTATATACATCTCTGACCAATTGCTTTCAGTTTCTGTGCTTATCTCT |
| | | CTACGACCTTTGTAACACACAGTGAACCAGCGCTGGCCCATGGATACACTCTAGTA |
| | | GCCCCAATCTAGCTAAGGCAGCCCCTTATAGTTAATCAATCCTGTCAAACAGGAAA |
| | | GGCTGGCAAAACCACTGGTCTGCATGTACTTTGTCCTTTACACAAGGAAGGATGCA |
| | | AACGTGGAAAACTGAGTGGACATGGTGTTCAGGAGATTGAGGCTCAGCTAAATTC |
| | | CAGCTTATTTACCTGCAGTTGCTTACAAAGTGTTTGGACATAATTGTGTAAAGCTA |
| | | GGGTTTTTTTTCTGGTTTTTAAAACAGGTAAAGGATGTCACAGCACCACTTAATAA |
| | | CATTTCTTCTGAAGTCCAGATTTTAACATCTGATGCCAATAAATTAACTGCTGAGA |
| | | ACATCACTAGTGCTACGCGAGTGGTTGGACAGATATTCAACACTTCCAGAAATGCT |
| | | TCACCTGAGGTAAAACTCACAGAGCTTTAAAAAAAATTTTTTTTTTATTTTTAGAG |
| | | GGGGTGTTGGGAAAGAAAGAGAGATGTCCTGAATACCACTCCTTATTCCACAATA |
| | | TGGTTTGAGCATGGTTTAAGTTGCTTAAAGGCATTTTGCATAGCACTCCTGAGAGA |
| | | CGCTCAAAGATTAAAAAAAGGGCCTCTTGTCCAAATATTTTATTTTAGGATATTCC |
| | | TAATGATCATTA |
| SUM8 | 8 | GCTAGAAAAGTCCAAGAATCCAGGAACGGCCCCACATCAGGCAAGGATTAGAGA |
| | | AGGGACAGCGGCTCCGTAGCTGGATCTCCTAGACTCAGAGGAGTTTCCAAACAAC |
| | | AGGCGTCTTTTTCAGTATTATAACAGAATTAACGGTGGCATCCAACATTTTCCTAT |
| | | GCACTGGTTTTAAGCTTATTTCTATAAAACCTCATTTAATTCTCACAAGAACATCAC |
| | | TGGGAACGCACTAACCCTTTACAGGCACGAGGAAACTGAGACAAGGAGAGATTAA |
| | | GTAACTTGCCCAATATTTTACTGCGAGGAGCAGGCAGGACTGGCTGGTAGGTGCGT |
| | | GAGGACGGCTAGGGCAGCAGACCCTCAACCTCGAAGGACGACGCGAGCCCGCCTC |
| | | CCACGCAAGAGGTTCTGCCGAGGGGACGCAAACTCCCGCAGCCTCCCAACCCCGC |
| | | CCTCCAGCGCTGGCCGCGGCTTCTCTTCCCCAAGCCAAGGACGGGTTCATTGCTGG |
| | | CCCCACGCCTAGGCAAAGAGGGCTCGGAGAAGTCTTGGAGACCCGGAGCTGACTC |
| | | ACCTCCCTGGGAGATATCTCAGATCCGACCTCGCCTTCCTGGAGCGGAAGTGCCTC |
| | | CGAGTGCCCACGGTTCGTGGACTCCACTTCCCAGAATTCCTAAGCGGAAGCGTCTC |
| | | AGAGTTCTCTCACTCCTGGACTACAGTTCCCAGAATGCTCCAAGGGCAGCGCTTCC |
| | | CACCAGCTGGGCTTTGTTTTGACTCAAGCCGGTTGGGTTTGAGCCACTTCTCTGGTG |
| | | CTACTACTGTGAGATTCGTTTTTGTTTTATTCATTTTTAAAAGCTCATGAGAAAATA |
| | | ATTGTAAAATTTTTAAAGTGATGACATATAAAAATGAAAAGTGCAGCTTGTATTAT |
| | | TAAAATAAGTTAGAAACCAAAATTTTTCGCAGAAGCAGAGGGTAGGCTAAAATGG |
| | | GAAGCGTAAAAGTGACTTAGGCTGAGCTCCGATGGACGCTGAACGTTAAGGAACA |
| | | CCAGATCTCAAACTTGTGTCAAATGCCGGCTCCCCAAATATCATCAACTCAACCTG |
| | | TTGATAAGACAAAAAACAGAATTTATTGTTGATTGCAGTAATGGAGAACAACACT |
| | | TCGACGGAGCGTTGGCACCTTCACAGAGCAAAGTGTCAAGGCAAGGTTGGAACTG |
| | | ATTGAGAACTGGATGTTTAAGGCGGGTCTTTCAATGCAAGGGATTTTATTAGTATT |
| | | GCATAATGATCATGATACTACAGTCCAGGATTGGTGGATATAGCAAGGATTTTTGA |
| | | AGCAAAGAATCTTAAATTGCAATCTATTGACACTTTCTTTGGAAGAGATTGAAAAT |
| | | TTGATGGGTCTCTGTGGAAGTTCCCATAATGAACAATTAAACAGTTTGCTTGGGCA |
| | | GGAATCTCCTGGAATAAAGGCCTGCTGATGAAGACAGCTGAACAATCATGTTAGT |
| | | GCCGAAAGTAAATTGTGATGAAACGGGAAAAGTTCACTTCTCCCTCTCCCAGGGC |
| | | ATGTG |
| SUM9 | 9 | ACGATCTCGGCTCCCTGAAACCTACACCTCCTGGATTCAACCGATTCTCCAGCCTC |
| | | AGCCTCCAGAGTAACTGGGATTACAGGCGCGCACCACCACGCCCAGCTAATTTCTG |
| | | AATTTTTAAATAGAAACAGGGTTTCACCATGTTGCCCAGGCTGGTCTCGAACTCCT |
| | | GACCTCAGGTGATCCGCCCGCTTCGGCCTCCCAAAGTGCTGGGATTACAGGCGTGA |
| | | GCCACCGTGCCGGTCCTCCAAGACAGTTTTAAAAAGGAAAATACTCATGACCAGC |
| | | TTGGGGAAAGGTGGCAAAAACCTCGTTTCTACAAAAACAAAAATAAAAATAAAAA |
| | | TAAAAATTTAAGTAAAATATCTCAAGAACTTTTTATATTGTGTTGAAATAGTAATG |
| | | TTTTGGACATATTGGGTTAAATTGTATTTAAAAATCAATTTCATCTGTTTCATTTTA |
| | | CCTTTAGTGTGGTTACTAGAAAATTTATAATTACATTTGCGGCTCACATGAGACTA |
| | | GCATTTTATTTCCATTGGACAGCGCTGGCTGAGAACAAAACCTAACCCTCTGTGCC |
| | | GCCCTCGCGGCCGGGATGCGGTGCGCCCCGGGCCTCCCCATTCGGAAAACGAGGA |
| | | GCCTGGGCGCAACGGGGCCGGCAGCCGCCTTAGCTACCCGGCTGGCGCTGGGGAC |
| | | CCCGTCCCCCGAACTGCACACCGCGTGGCGGGGGAAGGGGTCCCAGCCCTCGCGG |
| | | AGCACCTTTGGCCCAGGGCGGGAGCTCCGAGCGCCGGCTTCCCGCGCCCCGCACG |
| | | GCCCGGCCCAGCCTGGAAGAAGGCCTGAGGAAGGCCCGACCGCGCCGCCCGCGCC |
| | | CTTAGGCCCGGCCCGAATGCTGGAAAGCCCGCCGGCCCGCCCTCGGCTCGGTGAG |
| | | CCTCGGCTCACCTCAGCTGCCGGTCGTACTTCTGCTCCTTGAGCAGCTTTCCCAGCT |
| | | GCGCCATGGCCGCGCCTGCCGCGCGGAAAACAGCCGAGCCCCTGCGGAGCGCCGC |
| | | CACCAGCTCCACAAGCGCGCAGGCGCACTGAGCGCCCCTTCGCCGCTACCGTCTCC |
| | | TGCGCGCTGCCTGCAGGGGGCGCCGCACTGCACTGCGCACGCGTCGCCGGTACGC |
| | | CGGGGCCTAAGCGGCCCTGGTACCCGAGCGGGTCTGGGGCTCTTGGTCCCCATCGG |
| | | CCAGAAAGGCAGACCCGCTAGCCTGCCAATTTGCCCTCTGTCCATGCAGTAATTGG |
| | | GGGCGACTGGGGCATTGGAATCAGGCATACTTGTATTTCGACCACTTAACTGGCTG |
| | | TGAGATCTACTCTTTACCTCTCAAATCCTCAGTGTGCTCACCTGTGCAGTGGGGAT |
| | | AAATGGGTCCCAAGTGTCTTTTGACCTTAAATGAGATTTTGAATACAGGAGGCCAG |
| | | ACTCCCCCCAAATCTTGCATGAATAATAACTTTCATTTTGATGATCATTATTTTTCA |
| | | CCCATTTCTTCCTCTCATGAAAAGCCCACAAGCCTATTTTGAATCACAAAGCTAGG |
| | | GGCTCCCTGCTGCAGATGCAGACAAGTCCAGGGCTCTGTTCTTGGAGAGTTTAGTC |
| | | TCATGAAGGAAGAGGGACAGCAAACAAGCACTTACCAGACAATGTGGCCTTCCTC |
| | | TAGATAGAGGGAAGCAGGGTGCTCAGGGGTGCACAGAAGAACCT |
| SUM10 | 10 | GTGATTAGCCAGAGCTGATAACCTGTTATTAGGGGATTTTCTCTGTGAATAACCTT |
| | | GACGCTGGAGTGAGGAAGGGCTTAGCAGCATTGAAATAAACGCTTCCCATTGGCT |
| | | GGAGAAATTTCCTCGTGAGCCACTCCTCCTTTAATTTATCCCACCCTTTCTCCTTTC |
| | | CTCCCTTCTACCCATTTTTGTTATAATTATTTAGAATTAATCATACAGTTCCCTTTCA |
| | | TTCATCCAAGCTATTACTCTGTTGCAACAAAGGTCAGGATGAAAAGGGCAGCGCG |
| | | GTTGGCAGCTCCATATTTTTACTTCCTCTTGGTGTAAAGTTTTACTGAATAAAGGGA |
| | | GTGCTACAGTGAGACGCTCAGACGAGGGGTAAATAGGAAATGAGCCAGTGCCCAG |
| | | CTGGGCTCAGCCTGGTCCACTCCACATTGCCAGCCATCAGACTTTCTAGAAAACAC |
| | | AGGCTGAAGGCACCCCTGCTGCCTCTCAAACTCTCCGAGGTTTTTTGTTTTGTTTTG |
| | | TGTGTGTGTGTGTGTGTGTGTGTGTGTGTGTGGTGCAATGCTCACACATGAAGAAG |
| | | GCCAAACCTCAAGCTTTCTTCACGTACCTGTACATTTCAGTCTCTGAAAGAAAGCA |
| | | CTGTCTACTACGAATGTTTTATTCTGCCATCTGCCTGGTGGAAATTACTGAGGGAA |
| | | ACAGCTGGTTCAGCACTTCCGCCACTAGCTTTTATAAAGGAAGAACCAAAGAACT |
| | | GTTCCTCATCAGTTGTGAAGCCTA |

### Expression Vectors and Expression Vector Delivery Agents

Expression vectors of the present disclosure can be nucleic acids (e.g., nucleic acids of double-stranded DNA) that include an S/MAR. As those of skill in the art will appreciate, expression vectors can be used to achieve and/or control expression of a heterologous coding sequence. In various embodiments, an expression vector of the present disclosure is a nucleic acid that does not include a coding sequence operably linked with the S/MAR, but into which a coding sequence can be added or inserted such that it is operably linked with the S/MAR. In various embodiments, an expression vector of the present disclosure is a nucleic acid that includes an S/MAR operably linked with a heterologous sequence. In various embodiments an S/MAR is operably linked with a heterologous sequence, where the heterologous sequence is a coding sequence and the S/MAR controls or contributes to control of the level, duration, and/or tissue specificity of expression of the coding sequence. In various embodiments an S/MAR is operably linked with a heterologous sequence, where the heterologous sequence includes a coding sequence and a promoter operably linked with the coding sequence, where the S/MAR controls or contributes to control of the level, duration, and/or tissue specificity of expression of the coding sequence. Those of skill in the art will appreciate that inclusion of an S/MAR can modulate, control, and/or contribute to control of, the level, duration, and/or tissue specificity of expression of a coding sequence operably linked with a promoter in that the level, duration, and/or tissue specificity of expression of the coding sequence from a construct in which the S/MAR is operably linked with the coding sequence and/or heterologous sequence differs from the level, duration, and/or tissue specificity of expression of the coding sequence from a construct in which the S/MAR is not operably linked with the coding sequence and/or heterologous sequence.

Expression vectors can be engineered to encode and/or used to express any of a wide variety of expression products. Those of skill in the art will appreciate that expression vectors are of general utility in various contexts, including laboratory and therapeutic or clinical contexts, in which expression of a coding sequence is desired. In particular embodiments, expression vectors of the present disclosure are particularly useful for expression of an expression product operably linked with an S/MAR of the present disclosure in one or more of liver, retina, skeletal muscle, and neurons or the central nervous system.

In accordance with the present disclosure, nucleic acids and/or expression vectors of the present disclosure can be engineered to include an S/MAR as disclosed herein and a means of positioning a coding sequence in operable linkage with the S/MAR. In certain embodiments, a nucleic acid of the present disclosure that includes an S/MAR operably linked with a coding sequence is synthesized, which nucleic acid can be or can be inserted into an expression vector, where such synthesis can be achieved according to any of the various nucleic acid synthesis methods known in the art. In certain embodiments, a synthesized nucleic acid of the present disclosure includes an S/MAR operably linked with a coding sequence, the synthesized sequence optionally further including one or more of a promoter, a 3′ UTR of a coding sequence, a 5′ UTR of a coding sequence, a polyadenylation signal, or other regulatory sequence, In certain embodiments, a synthesized nucleic acid of the present disclosure includes an S/MAR operably linked with a coding sequence and further includes, without limitation, a promoter operably linked to the coding sequence, In various embodiments an expression vector of the present disclosure includes such a synthesized nucleic acid. In certain embodiments, a nucleic acid and/or expression vector of the present disclosure can be synthesized according to any of the various nucleic acid synthesis methods known in the art. In certain embodiments, an expression vector can include an S/MAR and a restriction site, where the restriction site is positioned such that insertion of a coding sequence (e.g., insertion of a nucleic acid including a coding sequence) at the restriction site would place the coding sequence in operable linkage with the S/MAR.

In various embodiments, heterologous and/or coding sequence is inserted or otherwise positioned upstream of an S/MAR such that it is operably linked with S/MAR. In various embodiments, an S/MAR is no more than 5 kb upstream of heterologous and/or coding sequence or position at which such a sequence is inserted or otherwise positioned, e.g., no more than 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp upstream of the heterologous and/or coding sequence or position at which such a sequence is inserted or otherwise positioned. In various embodiments, an S/MAR is upstream of the heterologous and/or coding sequence or position at which such a sequence is inserted or otherwise positioned by a number of base pairs having a lower bound of 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp and an upper bound of 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, or 100 bp. In various embodiments, an S/MAR is upstream of the heterologous and/or coding sequence or position at which such a sequence is inserted or otherwise positioned by a number of base pairs having a lower bound of 50 bp, 40 bp, 30 bp, 25 bp, 20 bp, 15 bp, 10 bp, 5 bp, 4 bp, 3 bp, 2 bp, 1 bp, or 0 bp and an upper bound of 100 bp, 90 bp, 80 bp, 70 bp, 60 bp, 50 bp, 40 bp, 30 bp, 25 bp, 20 bp, 15 bp, 10 bp, or 5 bp.

In various embodiments, a heterologous and/or coding sequence is inserted or otherwise positioned downstream of an S/MAR such that it is operably linked with S/MAR. In various embodiments, an S/MAR is no more than 5 kb downstream of a heterologous and/or coding sequence or position at which such a sequence is inserted or otherwise positioned, e.g., no more than 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp downstream of the heterologous and/or coding sequence or position at which such a sequence is inserted or otherwise positioned. In various embodiments, an S/MAR is downstream of the heterologous and/or coding sequence or position at which such a sequence is inserted or otherwise positioned by a number of base pairs having a lower bound of 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp and an upper bound of 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, or 100 bp. In various embodiments, an S/MAR is downstream of the heterologous and/or coding sequence or position at which such a sequence is inserted or otherwise positioned by a number of base pairs having a lower bound of 50 bp, 40 bp, 30 bp, 25 bp, 20 bp, 15 bp, 10 bp, 5 bp, 4 bp, 3 bp, 2 bp, 1 bp, or 0 bp and an upper bound of 100 bp, 90 bp, 80 bp, 70 bp, 60 bp, 50 bp, 40 bp, 30 bp, 25 bp, 20 bp, 15 bp, 10 bp, or 5 bp.

In various embodiments, a restriction site is positioned upstream of an S/MAR such that a coding sequence inserted at the restriction site is operably linked with S/MAR and the S/MAR is upstream of the restriction site. In various embodiments, an S/MAR is no more than 5 kb upstream of a restriction site, e.g., no more than 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp upstream of the restriction. In various embodiments, an S/MAR is upstream of the restriction site by a number of base pairs having a lower bound of 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp and an upper bound of 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, or 100 bp. In various embodiments, an S/MAR is upstream of the restriction site by a number of base pairs having a lower bound of 50 bp, 40 bp, 30 bp, 25 bp, 20 bp, 15 bp, 10 bp, 5 bp, 4 bp, 3 bp, 2 bp, 1 bp, or 0 bp and an upper bound of 100 bp, 90 bp, 80 bp, 70 bp, 60 bp, 50 bp, 40 bp, 30 bp, 25 bp, 20 bp, 15 bp, 10 bp, or 5 bp.

In various embodiments, a restriction site is positioned downstream of an S/MAR such that a coding sequence inserted at the restriction site is operably linked with S/MAR and the S/MAR is downstream of the restriction site. In various embodiments, an S/MAR is no more than 5 kb downstream of a restriction site, e.g., no more than 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp downstream of the restriction. In various embodiments, an S/MAR is downstream of the restriction site by a number of base pairs having a lower bound of 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp and an upper bound of 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, or 100 bp. In various embodiments, an S/MAR is downstream of the restriction site by a number of base pairs having a lower bound of 50 bp, 40 bp, 30 bp, 25 bp, 20 bp, 15 bp, 10 bp, 5 bp, 4 bp, 3 bp, 2 bp, 1 bp, or 0 bp and an upper bound of 100 bp, 90 bp, 80 bp, 70 bp, 60 bp, 50 bp, 40 bp, 30 bp, 25 bp, 20 bp, 15 bp, 10 bp, or 5 bp.

Expression vectors of the present disclosure can be engineered to encode and/or express a wide variety of diverse expression products. In certain embodiments, an expression vector of the present disclosure includes a nucleic acid sequence synthesized to include an S/MAR operably linked with a coding sequence, the synthesized sequence optionally further including one or more of a promoter, a 3′ UTR of a coding sequence, a 5′ UTR of a coding sequence, a polyadenylation signal, or other regulatory sequence, In certain embodiments, an expression vector of the present disclosure includes a nucleic acid sequence synthesized to include an S/MAR operably linked with a coding sequence, the synthesized sequence further including, without limitation, a promoter operably linked to the coding sequence, In certain embodiments, an expression vector of the present disclosure includes a restriction site positioned such that insertion of a coding sequence at the restriction site places the coding sequence in operable linkage with an S/MAR, and such insertion is used to operably link a coding sequence of interest with the S/MAR. In certain embodiments, an expression vector of the present disclosure that includes an S/MAR operably linked with a coding sequence can be synthesized according to any of the various nucleic acid synthesis methods known in the art.

Exemplary expression products can include, without limitation, one or more of a polypeptide, peptide, RNA, ribozyme, aptamer, or other expression product. In various embodiments, a polypeptide encoded and/or expressed by an expression vector of the present disclosure can be, for example, an enzyme, antibody, nanobody, Darpin, Ankyrin repeat, or structural protein. In various embodiments, an expression product encoded and/or expressed by an expression vector of the present disclosure is an inhibitory RNA, e.g., a gRNA, eRNA, siRNA, miRNA, or shRNA. In various embodiments, an expression product encoded and/or expressed by an expression vector of the present disclosure is a non-coding RNA. In various embodiments, an expression product encoded and/or expressed by an expression vector of the present disclosure is or includes a gRNA and a CRISPR editing enzyme, e.g., CRISPR-Cas9. In various embodiments, an expression product encoded and/or expressed by an expression vector of the present disclosure is a therapeutic expression product in that it is engineered, upon delivery to a subject, cell, or system, to treat a disease, disorder, or condition of the subject, cell, or system, or a symptom or characteristic thereof.

In various embodiments, an S/MAR is operably linked with a coding sequence in that it is upstream of the transcription start site (TSS) of the coding sequence. In various embodiments, an S/MAR operably linked with a coding sequence is no more than 5 kb upstream of a TSS of an operably linked coding sequence, e.g., no more than 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp upstream of the TSS. In various embodiments, an S/MAR operably linked with a coding sequence is upstream of the TSS of the coding sequence by a number of base pairs having a lower bound of 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp and an upper bound of 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, or 100 bp. In various embodiments, an S/MAR operably linked with a coding sequence is upstream of the TSS of the coding sequence by a number of base pairs having a lower bound of 50 bp, 40 bp, 30 bp, 25 bp, 20 bp, 15 bp, 10 bp, 5 bp, 4 bp, 3 bp, 2 bp, 1 bp, or 0 bp and an upper bound of 100 bp, 90 bp, 80 bp, 70 bp, 60 bp, 50 bp, 40 bp, 30 bp, 25 bp, 20 bp, 15 bp, 10 bp, or 5 bp. Accordingly, in certain embodiments, an S/MAR operably linked with a coding sequence is positioned immediately adjacent to the TSS of the operably linked coding sequence

In various embodiments, an S/MAR is operably linked with a coding sequence in that it is downstream of the transcription start site (TSS) or termination codon of the coding sequence. In various embodiments, an S/MAR operably linked with a coding sequence is no more than 5 kb downstream of a TSS or termination codon of an operably linked coding sequence, e.g., no more than 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp downstream of the TSS or termination codon. In various embodiments, an S/MAR operably linked with a coding sequence is downstream of the TSS or termination codon of the coding sequence by a number of base pairs having a lower bound of 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp and an upper bound of 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, or 100 bp. In various embodiments, an S/MAR operably linked with a coding sequence is downstream of the TSS or termination codon of the coding sequence by a number of base pairs having a lower bound of 50 bp, 40 bp, 30 bp, 25 bp, 20 bp, 15 bp, 10 bp, 5 bp, 4 bp, 3 bp, 2 bp, 1 bp, or 0 bp and an upper bound of 100 bp, 90 bp, 80 bp, 70 bp, 60 bp, 50 bp, 40 bp, 30 bp, 25 bp, 20 bp, 15 bp, 10 bp, or 5 bp. Accordingly, in certain embodiments, an S/MAR operably linked with a coding sequence is positioned immediately adjacent to the termination codon of the operably linked coding sequence

For the avoidance of doubt, in various embodiments, one or more nucleotides can be present between an S/MAR and a coding sequence and/or between as S/MAR and one or more other sequence features such as a promoter, a 3′ UTR of a coding sequence, a 5′ UTR of a coding sequence, a polyadenylation signal, or other regulatory sequence, which nucleotides can in some instances be referred to as intervening nucleotides. In various embodiments, intervening nucleotides between an S/MAR and a coding sequence can include all or a portion of one or more of a promoter, a 3′ UTR of a coding sequence, a 5′ UTR of a coding sequence, a polyadenylation signal, or other regulatory sequences, e.g., where the S/MAR and the promoter, UTR, polyadenylation signal, or other regulatory sequence are operably linked with a same coding sequence. In various particular embodiments, intervening nucleotides can include all or a portion of a promoter where the promoter and the S/MAR are operably linked with a same coding sequence.

In various embodiments, an S/MAR and a promoter are each individually and/or together operably linked with a coding sequence in that each is upstream of the transcription start site (TSS) of the coding sequence. In various embodiments, an S/MAR operably linked with a coding sequence is no more than 5 kb upstream of a promoter operably linked with a coding sequence, e.g., no more than 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp upstream of the promoter. In various embodiments, an S/MAR operably linked with a coding sequence is upstream of the promoter by a number of base pairs having a lower bound of 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp and an upper bound of 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, or 100 bp. In various embodiments, an S/MAR operably linked with a coding sequence is upstream of the promoter by a number of base pairs having a lower bound of 50 bp, 40 bp, 30 bp, 25 bp, 20 bp, 15 bp, 10 bp, 5 bp, 4 bp, 3 bp, 2 bp, 1 bp, or 0 bp and an upper bound of 100 bp, 90 bp, 80 bp, 70 bp, 60 bp, 50 bp, 40 bp, 30 bp, 25 bp, 20 bp, 15 bp, 10 bp, or 5 bp. Accordingly, in certain embodiments, an S/MAR operably linked with a coding sequence is positioned immediately upstream of and adjacent to a promoter of the operably linked coding sequence.

In various embodiments in which an S/MAR and a promoter are each individually and/or together operably linked with a coding sequence in that each is upstream of the transcription start site (TSS) of the coding sequence, the S/MAR is no more than 5 kb downstream of the promoter operably linked with a coding sequence, e.g., no more than 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp downstream of the promoter. In various embodiments, an S/MAR operably linked with a coding sequence is downstream of the promoter by a number of base pairs having a lower bound of 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp and an upper bound of 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, or 100 bp. In various embodiments, an S/MAR operably linked with a coding sequence is downstream of the promoter by a number of base pairs having a lower bound of 50 bp, 40 bp, 30 bp, 25 bp, 20 bp, 15 bp, 10 bp, 5 bp, 4 bp, 3 bp, 2 bp, 1 bp, or 0 bp and an upper bound of 100 bp, 90 bp, 80 bp, 70 bp, 60 bp, 50 bp, 40 bp, 30 bp, 25 bp, 20 bp, 15 bp, 10 bp, or 5 bp. Accordingly, in certain embodiments, an S/MAR operably linked with a coding sequence is positioned immediately downstream of and adjacent to a promoter of the operably linked coding sequence.

In various embodiments, an S/MAR and a 3′ UTR are each individual and/or together operably linked with a coding sequence in that each is downstream of the termination codon of the coding sequence. In various embodiments, an S/MAR operably linked with a coding sequence is no more than 5 kb downstream of a 3′ UTR operably linked with a coding sequence, e.g., no more than 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp downstream of the 3′ UTR. In various embodiments, an S/MAR operably linked with a coding sequence is downstream of the 3′ UTR by a number of base pairs having a lower bound of 2 kb, 1500 bp, 1000 bp, 750 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 25 bp, 20 bp, 15 bp, 10 bp, 5 bp, 4 bp, 3 bp, 2 bp, 1 bp, or 0 bp and an upper bound of 5 kb, 4 kb, 3 kb, 2 kb, 1500 bp, 1000 bp, 750 bp, 500 bp, 450 bp, 400 bp, 350 bp, 300 bp, 250 bp, 200 bp, 150 bp, or 100 bp. In various embodiments, an S/MAR operably linked with a coding sequence is downstream of the 3′ UTR by a number of base pairs having a lower bound of 50 bp, 40 bp, 30 bp, 25 bp, 20 bp, 15 bp, 10 bp, 5 bp, 4 bp, 3 bp, 2 bp, 1 bp, or 0 bp and an upper bound of 100 bp, 90 bp, 80 bp, 70 bp, 60 bp, 50 bp, 40 bp, 30 bp, 25 bp, 20 bp, 15 bp, 10 bp, or 5 bp. Accordingly, in certain embodiments, an S/MAR operably linked with a coding sequence is positioned immediately downstream of and adjacent to the 3′ UTR of the operably linked coding sequence

The terms upstream, as used herein, can refer to the relative positions and/or number of nucleotides between the proximal termini of two sequences, e.g., the 3′-most nucleotide of a first sequence and the 5′-most nucleotide of a second sequence positioned 3′ of the first in the same nucleic acid. The terms downstream, as used herein, can refer to the relative positions and/or number of nucleotides between the proximal termini of two sequences, e.g., the 3′-most nucleotide of a first sequence and the 5′-most nucleotide of a second sequence positioned 5′ of the first in the same nucleic acid. In accordance with convention, comparison of two sequences in a double-stranded nucleic acid can be based on positions within a single particular strand of the nucleic acid.

In various embodiments, an S/MAR is operably linked with a coding sequence in that it is present in an intron of the coding sequence. Those of skill in the art will appreciate that introns can be identified empirically by transcript analysis or can be directly identified from sequences using any of a wide variety of predictive algorithms known to those of skill in the art.

In various embodiments, an expression vector of the present disclosure is or includes RNA, DNA, or a nucleic acid analog thereof. In various embodiments, an expression vector of the present disclosure can be single stranded or double stranded. In various embodiments, an expression vector of the present disclosure can be linear or circular. In various embodiments, an expression vector of the present disclosure can be a cDNA molecule. Examples of expression vectors include, without limitation, plasmids, viral genomes, linear single-stranded DNA, and linear double-stranded DNA.

The present disclosure includes the recognition that S/MARs can have a size that facilitates inclusion in various expression vectors that are characterized by limited capacity. For example, the capacity of an adeno-associated viral genome is about 4 kb. In other examples, the capacity of an expression vector is instead or additionally limited by factors other than intrinsic carrying capacity. For example, in various embodiments, production of a DNA plasmid can be inhibited by increasing plasmid size, where use of an S/MAR of limited size can decrease total plasmid size and thereby significantly increase production titer and/or decrease the dose required for administration to a subject, cell, or system, e.g., the therapeutically effective dose. In various embodiments in which an expression vector has limited capacity, the present disclosure includes that the expression vector can include an S/MAR selected at least in part based on its length, e.g., where an S/MAR included in the limited capacity expression vector is an S/MAR having a length of no greater than 2500 bp, 2000 bp, 1500 bp, 1000 bp, or 500 bp. In various embodiments, an S/MAR included in a limited capacity expression vector is an S/MAR having a length that is between a lower bound of 500 bp, 1000 bp, or 1500 bp, and an upper bound of 1500 bp, 2000 bp, or 2500 bp.

In various embodiments, a nucleic acid or expression vector including an S/MAR can be modified to reduce the number of CpG dinucleotide motifs present in the nucleic acid or expression vector, or in the S/MAR, at least in part because CpG content can be immunostimulatory. In various embodiments, an express vector can be modified to remove the bacterial backbone. Such approaches are exemplified, e.g. in Argyros et al., 2011, which is incorporated herein by reference in its entirety and with respect to expression vector modifications. In various embodiments, inclusion of an S/MAR in a nucleic acid sequence and/or expression vector can reduce the number of CpG dinucleotide motifs per unit length as compared to a reference (e.g., a reference nucleic acid sequence or expression vector that does not include the S/MAR) at least in part because S/MARs can be AT rich and/or low in CpG dinucleotide motifs.

In various embodiments, an expression vector of the present disclosure is associated with one or more agents that facilitate delivery to a subject, cell, or system (a “delivery agent”). Examples of expression vectors associated with a delivery agent, can include for example a viral genome encapsidated within a corresponding viral particle, a phage, a nanoparticle, or a liposome.

In certain embodiments, an expression vector of the present disclosure is associated with a delivery agent that is a mini-nucleosome core protein. Mini-nucleosome core proteins are described, for example, in PCT Publication WO 2020/097235, entitled Mini-Nucleosome Core Proteins and use in Nucleic Acid Delivery, filed as PCT/US19/60119 on Nov. 6, 2019, which is incorporated herein by reference in its entirety and particularly with respect to mini-nucleosome core proteins and sequences thereof. In certain embodiments, an expression vector of the present disclosure is associated with a delivery agent that is a modified mini-nucleosome core protein. Mini-nucleosome core proteins are described, for example, in International Application No. PCT/US21/26917, entitled Modified Mini-Nucleosome Core Proteins and use in Nucleic Acid Delivery, filed Apr. 12, 2021, which is incorporated herein by reference in its entirety and particularly with respect to modified mini-nucleosome core proteins and sequences thereof.

While mini-nucleosome core proteins are disclosed in WO 2020/097235, which is incorporated herein by reference in its entirety and particularly with respect to mini-nucleosome core proteins and sequences thereof, a partial discussion of mini-nucleosome core proteins is provided below by way of non-limiting exemplification.

In various embodiments, a mini-nucleosome core protein is an engineered polypeptide including (i) a nucleic acid binding domain and (ii) at least one of a nucleic acid release domain, a nuclear localization signal, a stability domain, an oligomerization domain, and a targeting domain, e.g., as disclosed in WO 2020/097235. In various such embodiments, the nucleic acid binding domain is a nucleic acid binding domain derived from a histone polypeptide sequence. In various such embodiments, the nucleic acid binding domain is or includes the amino acid sequence KRHRK (SEQ ID NO: 11). In various such embodiments, the nucleic acid binding domain is or includes an amino acid sequence that includes KRHRK (SEQ ID NO: 11), RRRRR (SEQ ID NO: 12), RRLARR (SEQ ID NO: 13), KKAKAAAKPKK (SEQ ID NO: 14), KKDGKKRKR (SEQ ID NO: 15), KKKLK (SEQ ID NO: 16), KKRIRK (SEQ ID NO: 17), RKKSK (SEQ ID NO: 18), KKPKK (SEQ ID NO: 19), or a combination thereof.

In various embodiments, the engineered polypeptide includes a targeting domain. In various such embodiments, the targeting domain is a cell attachment targeting domain, a beta galactose binding domain, a fucose binding domain, a heparin binding domain, a sialic acid binding domain, a glycoprotein binding domain, a carbohydrate binding domain, a lysophosphatidic acid binding domain, a cAMP binding domain, a hyaluronan binding domain, a chondroitin sulfate binding domain, an integrin binding domain, a nucleolin binding domain, a collagen binding domain, a clathrin binding domain, a Fc receptor binding domain, an actin binding domain, an endocytosis motif, a nuclear localization signal, or a combination thereof. In various embodiments, the cell attachment targeting domain is or includes an amino acid sequence that includes WGREERQ (SEQ ID NO: 20), NTQIH (SEQ ID NO: 21), WNNKTPH (SEQ ID NO: 22), TPH (SEQ ID NO: 23), VNRWS (SEQ ID NO: 24), XBBBXXBX (SEQ ID NO: 25), ARKKAAKA (SEQ ID NO: 26), QRR (SEQ ID NO: 27), SRR (SEQ ID NO: 28), WEPSRPFPVD (SEQ ID NO: 29), HRRTRKAPKRIRLPHIR (SEQ ID NO: 30), KRTGQYKLGSKTGPGQK (SEQ ID NO: 31), KKTK (SEQ ID NO: 32), KLRSQLVKK (SEQ ID NO: 33), RRRCGQKKK (SEQ ID NO: 34), BX(7)B (SEQ ID NO: 35), RIQNLLKITNLRIKFVK (SEQ ID NO: 36), KKEKDIMKKTI (SEQ ID NO: 37), KGE (SEQ ID NO: 38), RGD (SEQ ID NO: 39), RGDS (SEQ ID NO: 40), TTVVNPKYEGK (SEQ ID NO: 41), ERMSQIKRLLS (SEQ ID NO: 42), WRHRARS (SEQ ID NO: 43), GFOGER (SEQ ID NO: 44), LFDLM (SEQ ID NO: 45), WGREERQ (SEQ ID NO: 46), QSTEKRG (SEQ ID NO: 47), LPNTG (SEQ ID NO: 48), or a combination thereof.

In various embodiments, the targeting domain is an internalization domain. In various embodiments, the internalization domain is or includes an amino acid sequence that includes FXDXF (SEQ ID NO: 49), PPSY (SEQ ID NO: 50), FEDNFVP (SEQ ID NO: 51), YIRV (SEQ ID NO: 52), YADW (SEQ ID NO: 53), YTQV (SEQ ID NO: 54), KKRPKP (SEQ ID NO: 55), SSDDE (SEQ ID NO: 56), RRASS (SEQ ID NO: 57), (YXXL)2 (SEQ ID NO: 58), LPLTG (SEQ ID NO: 59), LAFTG (SEQ ID NO: 60), or a combination thereof.

In various embodiments, the targeting domain is a cell-type specific targeting domain. In various embodiments, the cell-type specific targeting domain is or includes an amino acid sequence that includes ASSLNIA (SEQ ID NO:61), KKEEEKKEEEKKEEE (SEQ ID NO: 62), LIFHKEQ (SEQ ID NO: 63), KFNKPFVFLI (SEQ ID NO: 64), QPEHSST (SEQ ID NO: 65), EYHHYNK (SEQ ID NO: 66), NGR (SEQ ID NO: 67), GEKGEP (SEQ ID NO: 68), KTKKK (SEQ ID NO: 69), KALKKK (SEQ ID NO: 70), KGKKK (SEQ ID NO: 71), CSVTCG (SEQ ID NO: 72), LRE (SEQ ID NO: 73), YKYNLNGRES (SEQ ID NO: 74), YRSL (SEQ ID NO: 75), KGGK7 (SEQ ID NO: 76), KKKQYTSIHHG (SEQ ID NO: 77), KDEL (SEQ ID NO: 78), LADQDYTKTA (SEQ ID NO: 79), DDNN (SEQ ID NO: 139), SAVTTVVN (SEQ ID NO: 140), or a combination thereof.

In various embodiments, the engineered polypeptide includes a poly-arginine domain.

In various embodiments, the engineered polypeptide includes a nuclear internalization signal or a nuclear import machinery binding domain. In various embodiments, the nuclear internalization signal or a nuclear import machinery binding domain is or includes an amino acid sequence that includes KKKYKLK (SEQ ID NO: 80), KKRKLIE (SEQ ID NO: 81), TRSK (SEQ ID NO: 82), HRKRKR (SEQ ID NO: 83), NKRKRK (SEQ ED NO. 84), AEKSKKK (SEQ ID NO: 85), RKSK (SEQ ID NO: 86), KRVK (SEQ ID NO: 87), KRK (SEQ ID NO: 88), LQQTPLHLAVI (SEQ 1ID NO: 89), RRPR (SEQ ID NO: 90), PRPR (SEQ ID NO: 91), RPPP (SEQ ID NO: 92), RKKRKGK (SEQ ID NO: 93), PAAKRVKLD (SEQ ID NO: 94), KLKIKRPVK (SEQ ID NO: 95), PKKKRKV (SEQ ID NO: 96), QRKRQK (SEQ ID NO: 97), DSPE (SEQ ID NO: 98), FQVT (SEQ ID NO: 99), QSTEKRG (SEQ ID NO: 100), RQGLfD (SEQ ID NO: 101), Cyclic RKKH (SEQ ID NO: 102), or a combination thereof.

In various embodiments, the engineered polypeptide includes a nucleic acid release domain. In various embodiments, the nucleic acid release domain is or includes an amino acid sequence that includes GRKKRRQRRRPQ (SEQ ID NO: 103), KRH (SEQ ID NO: 104), KSVKKRSVSEIQ (SEQ ID NO: 105), NRRKKRAL (SEQ ID NO: 106), KFERQ (SEQ ID NO: 107), VRGP (SEQ ID NO: 108), NKDS (SEQ ID NO: 109), NRDN (SEQ ID NO: 110), ANNR (SEQ ID NO: 111), or a combination thereof.

In various embodiments, the engineered polypeptide includes a stability domain. In various embodiments, the stability domain is or includes an amino acid sequence that includes YTRF (SEQ ID NO: 112), GDAY (SEQ ID NO: 113), LLEE (SEQ ID NO: 114), RKKRRQRRR (SEQ ID NO: 115), YKSL (SEQ ID NO: 116), YENF (SEQ ID NO: 117), FQDL (SEQ ID NO: 118), YIGSR (SEQ ID NO: 119), IKVAV (SEQ ID NO: 120), or a combination thereof.

In various embodiments, the engineered polypeptide includes an oligomerization domain. In various embodiments, the oligomerization domain is selected from LIRERTE (SEQ ID NO: 121), LVEERTQ (SEQ ID NO: 122), IITFTK (SEQ ID NO: 123), ILFNK (SEQ ID NO: 124), PIRTLSK (SEQ ID NO: 125), YGNSPLHRFK (SEQ ID NO: 126), FFQKDR (SEQ ID NO: 127), KSRP (SEQ ID NO: 128), YVM (SEQ ID NO: 129), YMKM (SEQ ID NO: 130), RSSSFG (SEQ ID NO: 131), LKIRGRER (SEQ ID NO: 132), LKIRGRKR (SEQ ID NO: 133), HVIFKKVSR (SEQ ID NO: 134), RGPRV (SEQ ID NO: 135), RANVKHLK (SEQ ID NO: 136), YPKAG (SEQ ID NO: 137), and/or YPRTG (SEQ ID NO: 138), optionally where the oligomerization domain is positioned at the C-terminus of the engineered polypeptide.

In various embodiments, the engineered polypeptide includes a linker, optionally where the linker is a linker according to any one of GRKKRRQRRRPQ (SEQ ID NO: 141), KSVKKRSVSEIQ (SEQ ID NO: 142), NRRKKRAL (SEQ ID NO: 143), KFERQ (SEQ ID NO: 144), VRGP, NKDS, NRDN, ANNR, KRH, HL, RI, ET, GQ, RS, RD, RN, RC, RG, RL, DA, RA, GS, LT, FS, GL, SA, DP, GT, GC, RQ, LS, HA, FV, QH, EA, AL, LY, YL, GF, PS, RE, DP, PI, QS, and/or ND.

In various embodiments, an engineered polypeptide is an engineered polypeptide having at least 80% sequence identity (e.g., at least 80%, 85%, 90%, 95%, 96%, 97%, 98%, 99%, or 100%) sequence identity with an engineered polypeptide selected from Table 5.

TABLE 5

| | | Net | | | Iso- |
|---|---|---|---|---|---|
| | SEQ | charge | Number | Molecular | electric |
| Exemplary Mini-nucleosome core | ID | at | of | weight | point |
| protein sequences | NO. | pH7 | residues | (g/mol) | (pH) |
| KRHRKLREKRHRKLRRRRRLKRHRKKRHR | 144 | 22.4 | 34 | 4773.77 | 12.72 |
| KLREK | | | | | |
| KRHRKGSSLREKRHRKLRRRRRLKRHRKK | 145 | 22.4 | 40 | 5206.16 | 12.72 |
| RHRKLREGGSK | | | | | |
| KRHRKREGSSLREKRHRKNDLRRRRRLKR | 146 | 21.4 | 44 | 5720.65 | 12.46 |
| HRKKRHRKLREGGSK | | | | | |
| KKPKKREGSSLREKRHRKNDLRRRRRLKRH | 147 | 21.3 | 44 | 5624.6 | 12.38 |
| RKKRHRKLREGGSK | | | | | |
| RRLARRGSSLREKRHRKLRRRRRLKKPKKK | 148 | 22.2 | 41 | 5213.23 | 12.72 |
| RHRKLREGGSK | | | | | |
| KRHRKLREKRHRKLREKRHRKLKRHRKKR | 149 | 21.5 | 36 | 4984 | 12.48 |
| HRKLREK | | | | | |
| KRHRKRILREKRHRKLREARKRHRKLKRHR | 150 | 23.5 | 40 | 5480.61 | 12.56 |
| KKRHRKLREK | | | | | |
| KRHRKKGKKKKGEKGKKKLKGKKKLRRR | 151 | 25.1 | 35 | 4507.55 | 12.78 |
| RRRRQRR | | | | | |
| KRHRKAPAPKGKKKKGEKGKKKLKGKKK | 152 | 27.1 | 43 | 5294.51 | 12.79 |
| LKPKPRRRRRRRQRR | | | | | |
| KRHRKGGSGGKGKKKKGEKGKKKLKGKK | 153 | 25.1 | 41 | 4893.91 | 12.78 |
| KLARRRRRRRQRR | | | | | |
| KRHRKLREKRHRKRRRRRRRKRHRKLREK | 154 | 24.3 | 34 | 4906.85 | 12.84 |
| RRQRR | | | | | |
| KRHRKKRHRKKRVKKKRHRKRRRRRRDSL | 155 | 21.3 | 30 | 4141.02 | 12.86 |
| L | | | | | |
| KRHRKKRHRKYQKRVKKKRHRKSSSRRRR | 156 | 21.3 | 35 | 4693.55 | 12.64 |
| RRDSLL | | | | | |
| KRHRKKKEEEKKEEEKKEEEKRRRRRRRQ | 157 | 12.1 | 32 | 4473.09 | 11.61 |
| RRR | | | | | |
| KRHRKWRKKEEEKKEEEKKEEEKRIRRRRR | 158 | 14.1 | 36 | 5084.83 | 11.79 |
| RRQRRR | | | | | |
| KRHRKRGDKRHRKRRRRRKRHRKTPHKKK | 159 | 20.4 | 29 | 3964.72 | 12.82 |
| KRHRKFIRGDKRHRKRRRRRKRHRKLATPH | 160 | 20.4 | 33 | 4409.28 | 12.82 |
| KKK | | | | | |
| KRHRKRGDKRHRKRRRRRKRHRKGSSRNT | 161 | 22.4 | 36 | 4722.51 | 12.86 |
| PHQKKKK | | | | | |
| KRHRKRGDKRHRKLKRHRKRRRRKRHRKT | 162 | 22.5 | 33 | 4499.37 | 12.86 |
| PHKK | | | | | |
| KRHRKRGDKRHRKKRHRKKRHRKRGDKK | 163 | 19.4 | 30 | 3983.71 | 12.5 |
| TK | | | | | |
| KRHRKRGDKKRKKKKRGDKKRRRRRKKK | 164 | 21.1 | 32 | 4172.01 | 12.33 |
| PPSY | | | | | |
| KRHRKRKRKRKRRRRRKKKRASSLNIAKR | 165 | 24.1 | 32 | 4308.23 | 13.26 |
| RRR | | | | | |
| KRKKRKGKRLKRRREKRHRKRASSLNIAK | 166 | 20.1 | 32 | 4054.95 | 12.68 |
| KKK | | | | | |
| KRKKRRLKRKRKRRRRREKRHRKRRRQRR | 167 | 27.1 | 32 | 4618.63 | 13.01 |
| RKK | | | | | |
| KRKKRRKRKRRRRRKRHRKLRERKRRLRE | 168 | 24.1 | 31 | 4420.4 | 12.75 |
| KK | | | | | |
| KRKNGRKRKRKKRHRKKKKRRRRKRHRK | 169 | 28.2 | 34 | 4587.61 | 13.2 |
| NGRKKK | | | | | |
| KRHRKWRHRARSKRHRKKKKKKRKKRKG | 170 | 22.3 | 29 | 3902.77 | 13.03 |
| K | | | | | |
| KRHRKRGDKRHRKKKKNRRKKRALRKKR | 171 | 22.2 | 31 | 4047.92 | 12.73 |
| KGK | | | | | |
| KKRKRGGKTKKKAKKALKKKKKGKKKKR | 172 | 28 | 38 | 4541.77 | 12.87 |
| RRRKKAAPKK | | | | | |
| KKKAYPKALKKPKKKKKAYPKALKRRRRR | 173 | 29.1 | 43 | 5481.83 | 12.53 |
| KNRRKKRALKRHRK | | | | | |
| KTRSKKKKKRGDKKKKNRRKKRALNTQIH | 174 | 23.1 | 39 | 4725.78 | 12.4 |
| KKKKKAAPKK | | | | | |
| KGKKKKGEKGKKKLKGKKKLRRRRRSPKK | 175 | 23 | 34 | 4242.23 | 12.68 |
| RRQRR | | | | | |
| KRHRKLREKRHRKLRRRRRLKRHRKKRHR | 176 | 22.4 | 34 | 4773.77 | 12.72 |
| KLREK | | | | | |
| KRHRKLREKRHRKLREKRHRKLKRHRKKR | 177 | 21.5 | 36 | 4984 | 12.48 |
| HRKLREK | | | | | |
| KRHRKKGKKKKGEKGKKKLKGKKKLRRR | 178 | 25.1 | 35 | 4507.55 | 12.78 |
| RRRRQRR | | | | | |
| KRHRKLREKRHRKRRRRRRRKRHRKLREK | 179 | 24.3 | 34 | 4906.85 | 12.84 |
| RRQRR | | | | | |
| KRHRKKRHRKKRVKKKRHRKRRRRRRDSL | 180 | 21.3 | 30 | 4141.02 | 12.86 |
| L | | | | | |
| KRHRKKKEEEKKEEEKKEEEKRRRRRRRQ | 181 | 12.1 | 32 | 4473.09 | 11.61 |
| RRR | | | | | |
| KRHRKQSKKEEEKKEEEKKEEEKNQRRRR | 182 | 12.1 | 36 | 4930.53 | 11.61 |
| RRRQRRR | | | | | |
| KRHRKRGDKRHRKRRRRRKRHRKTPHKKK | 183 | 20.4 | 29 | 3964.72 | 12.82 |
| KRHRKRGDKRHRKLKRHRKRRRRKRHRKT | 184 | 22.5 | 33 | 4499.37 | 12.86 |
| PHKK | | | | | |
| KRHRKRGDKRHRKKRHRKKRHRKRGDKK | 185 | 19.4 | 30 | 3983.71 | 12.5 |
| TK | | | | | |
| KRHRKRGDKKRKKKKRGDKKRRRRRKKK | 186 | 21.1 | 32 | 4172.01 | 12.33 |
| PPSY | | | | | |
| KRHRKGGSRGDKKRKKKKRGDSSSKKRRR | 187 | 21.1 | 38 | 4634.43 | 12.33 |
| RRKKKPPSY | | | | | |
| KRHRKRKRKRKRRRRRKKKRASSLNIAKR | 188 | 24.1 | 32 | 4308.23 | 13.26 |
| RRR | | | | | |
| KRKKRKGKRLKRRREKRHRKRASSLNIAK | 189 | 20.1 | 32 | 4054.95 | 12.68 |
| KKK | | | | | |
| KRKKRRLKRKRKRRRRREKRHRKRRRQRR | 190 | 27.1 | 32 | 4618.63 | 13.01 |
| RKK | | | | | |
| KRKKRRKRKRRRRRKRHRKLRERKRRLRE | 191 | 24.1 | 31 | 4420.4 | pH 12.75 |
| KK | | | | | |
| KRKNGRKRKRKKRHRKKKKRRRRKRHRK | 192 | 28.2 | 34 | 4587.61 | 13.2 |
| NGRKKK | | | | | |
| KRKWRNGRKRKRQKRHRKKKKRARRRRK | 193 | 30.3 | 40 | 5422.54 | 13.26 |
| RHRKNGRKHKKK | | | | | |
| KRHRKWRHRARSKRHRKKKPKKRKKRKG | 194 | 21.3 | 29 | 3871.71 | 13.03 |
| K | | | | | |
| KRHRKPKPRIWRHRARSRDKRHRKKKPKK | 195 | 23.3 | 36 | 4734.73 | 12.78 |
| RKKRKGK | | | | | |

The present disclosure further includes a composition including: (i) at least one expression vector of the present disclosure, and (ii) at least one engineered polypeptide of the present disclosure. In certain embodiments, such a composition includes at least two of the engineered polypeptides, where a first of the engineered polypeptides is able to oligomerize with a second of the engineered polypeptides. In various embodiments, the ratio of the polynucleotides to the engineered polypeptides is between 1:3 and 1:2,000. In various embodiments, the ratio of the polynucleotides to the engineered polypeptides is between 1:3 and 1:1,000, between 1:3 and 1:500, between 1:3 and 1:200, between 1:3 and 1:100, or between 1:3 and 1:50. In various embodiments, the ratio of the polynucleotides to the engineered polypeptides is between 1:200 and 1:2,000, between 1:200 and 1:1000, or between 1:200 and 1:500. In various embodiments, the composition includes a pharmaceutical carrier. The present disclosure further includes a cell including composition including an engineered polypeptide as disclosed herein and an expression vector as disclosed herein.

In various embodiments, the present disclosure includes a method of condensing an expression vector of the present disclosure by contacting the expression vector with an engineered polypeptide as disclosed herein. In various embodiments, the present disclosure includes a method of neutralizing the charge of an expression vector that includes contacting the polynucleotide with an engineered polypeptide as disclosed herein.

In various embodiments, an engineered polypeptide as disclosed herein includes one or more amino acids that are pegylated, acetylated, methylated, glycosylated, phosphorylated, sumoylated, amidated, lipidated, prenylated, lipoylated, alkylated, acylated, glycated, nitrosylated, sulfated, carbamylated, carbonylated, neddylated, biotinylated, or ribosylated.

### Pharmaceutical Compositions and Administration

The present disclosure contemplates an expression vector composition that includes an expression vector and at least one delivery agent and/or at least one carrier. As disclosed herein, an expression vector composition may be in any form known in the art. An expression vector composition can be formulated such that it is suitable for laboratory, research, or therapeutic administration to a cell, system, or subject in vitro, ex vivo, or in vivo. An expression vector composition can be formulated such that it is suitable (e.g., pharmaceutically acceptable) for administration to animals. In various embodiments, an expression vector composition disclosed herein can be administered, or formulated for administration, to animals of commercial interest, livestock, and household pets such as dogs and cats. In various embodiments, an expression vector composition disclosed herein can be administered, or formulated for administration, to an animal that is a mammal, e.g., a human.

In various embodiments, an expression vector composition is a pharmaceutical composition that includes a pharmaceutically acceptable carrier. Formulations that include pharmaceutically acceptable carriers are well known to those skilled in the art, as is the development of suitable dosing and treatment regimens. Selection or use of any particular form may depend, in part, on the intended mode of administration and therapeutic application.

An expression vector composition can include one or more pharmaceutically acceptable carriers such as one or more solvents, dispersion media, coatings, antibacterial and antifungal agents, isotonic and absorption delaying agents, and the like that are physiologically compatible. Compositions of the present disclosure can include a pharmaceutically acceptable salt, e.g., an acid addition salt or a base addition salt.

In certain embodiments, an expression vector composition can be administered, or formulated for administration, by subcutaneous, intraocular, intravitreal, parenteral, intravenous, intramuscular, intrathecal, topical, oral, intraperitoneal injections, or nasal inhalation administration. Solutions including expression vectors can be prepared in sterile water or sterile saline and can also be suitably mixed with one or more surfactants, such as pluronic acid. Dispersions may also be prepared in glycerol, liquid polyethylene glycols, and mixtures thereof. Storage preparations may contain preservatives to prevent microorganisms from growing.

In various embodiments, an expression vector composition can be administered, or formulated for administration, as a sterile formulation for injection, and/or can be formulated in accordance with conventional pharmaceutical practices using distilled water for injection as a vehicle. Expression vector compositions suitable for injection can include sterile aqueous solutions or dispersions. Expression vector compositions suitable for injection can include physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Preferably, isotonic agents, for example, sugars or sodium chloride can be used in the formulations. For human administration, final preparations and compositions should meet sterility, pyrogenicity, and the general endotoxin levels, safety and purity standards as required by the US FDA and EU regulatory standards. Temperature and exposure to other proteins can alter the properties of loaded mini-nucleosomes.

In various embodiments, an expression vector composition of the present disclosure can be administered, or formulated for administration, as a solution, microemulsion, dispersion, liposome, or other ordered structure suitable for stable storage at high concentration. Sterile injectable solutions can be prepared by incorporating a composition disclosed herein in the required amount in an appropriate solvent with one or a combination of ingredients enumerated above, as required, followed by filter sterilization. Generally, dispersions are prepared by incorporating a composition disclosed herein into a sterile vehicle that contains a basic dispersion medium together with any other required ingredients known in the art. In the case of sterile powders for the preparation of sterile injectable solutions, methods for preparation include vacuum drying and freeze-drying that yield a powder of a composition disclosed herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

An expression vector composition as disclosed herein can be administered, or formulated for administration, as an immunoliposome composition. Such formulations can be prepared by methods known in the art.

In some embodiments, an expression vector composition can be administered, or formulated for administration, by intrapulmonary administration (e.g., administration via an inhaler or nebulizer) to a mammal such as a human. Methods for formulating such compositions are well known in the art. Dry powder inhaler formulations and suitable systems for administration of the formulations are also known in the art. Pulmonary administration may be oral and/or nasal. Examples of pharmaceutical devices for pulmonary delivery include metered dose inhalers, dry powder inhalers (DPIs), and nebulizers. For example, a composition disclosed herein can be administered to the lungs of a subject by way of a dry powder inhaler. In some embodiments, a composition disclosed herein can be intrapulmonarily administered by way of a metered dose inhaler.

In some embodiments, an expression vector composition can be administered, or formulated for administration, to the eye, e.g., in the form of a pharmaceutically acceptable solution, suspension or ointment. A preparation for use in treating an eye can be in the form of a sterile aqueous solution containing, e.g., additional ingredients such as, but not limited to, preservatives, buffers, tonicity agents, antioxidants and stabilizers, nonionic wetting or clarifying agents, and viscosity-increasing agents. A composition as disclosed herein can be administered topically to the eye of the subject in need of treatment (e.g., a subject afflicted with AMD) by conventional methods, e.g., in the form of drops, or by bathing the eye in a therapeutic solution, containing one or more compositions. A variety of devices for introducing drugs into the vitreal cavity of the eye may be appropriate, in certain embodiments, for administration of a composition as disclosed herein.

Expression vector compositions can be administered, or formulated for administration, by systemic or local delivery can be in the form of injectable or infusible solutions. An expression vector composition can be administered, or formulated for administration, by a parenteral mode (e.g., intravenous, subcutaneous, intraperitoneal, or intramuscular injection). As used herein, parenteral administration refers to modes of administration other than enteral and topical administration, usually by injection, and include, without limitation, injection, intravenous, intranasal, transnasal, intraocular, pulmonary, intramuscular, intraarterial, intrathecal, intracapsular, intraorbital, intracardiac, intradermal, intrapulmonary, transpulmonary, intraperitoneal, transtracheal, subcutaneous, transcutaneous, subcuticular, intraarticular, subcapsular, subarachnoid, intraspinal, epidural, intracerebral, intracranial, intracarotid and intrasternal injection and infusion. Administration can be systemic or local by intravenous injection, intramuscular injection, intraperitoneal injection, subcutaneous injection.

Expression vector compositions can be administered, or formulated for administration, parenterally, e.g., in the form of an injectable formulation comprising a sterile solution or suspension in water or another pharmaceutically acceptable liquid. For example, an expression vector composition can be formulated by suitably combining expression vector with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. Nonlimiting examples of oily liquid include sesame oil and soybean oil, and it may be combined with benzyl benzoate or benzyl alcohol as a solubilizing agent. Expression vector compositions can further include a buffer such as a phosphate buffer, or sodium acetate buffer, a soothing agent such as procaine hydrochloride, a stabilizer such as benzyl alcohol or phenol, and an antioxidant. An expression vector composition formulated for injection can be packaged in a suitable ampule.

Expression vector compositions can be administered, or formulated for administration, via electroporation, sonophoresis, intraosseous injections methods or by using gene gun. Expression vectors may also be implanted into microchips, nano-chips, or nanoparticles.

Expression vector compositions can be administered, or formulated for administration, by way of local administration. As used herein, “local administration” or “local delivery,” can refer to delivery that does not rely upon transport of the expression vector composition or expression vector composition to its intended target tissue or site via the vascular system. For example, an expression vector composition may be delivered by injection or implantation of an expression vector composition or by injection or implantation of a device containing the composition. In certain embodiments, following local administration in the vicinity of a target tissue or site, the composition, or one or more components thereof, may diffuse to an intended target tissue or site that is not the site of administration.

Expression vector compositions can be administered, or formulated for administration, by subcutaneous administration, e.g., accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

A suitable means of administration of an expression vector composition can be selected based on the condition or disease to be treated and/or upon the age and condition of a subject. An expression vector can be administered to a subject either alone or in combination with one or more other therapeutic modalities.

Dose and method of administration can vary depending on the weight, age, condition, and the like of a patient, and can be suitably selected as needed by those skilled in the art. Other factors such as solubility, bioavailability, half-life, shelf-life will be contemplated by one skilled in the art. A suitable dose of an expression vector composition disclosed herein, which dose is capable of treating or preventing a disorder in a subject, can depend on a variety of factors including, e.g., the age, sex, and weight of a subject to be treated, the condition or disease to be treated, and the particular expression vector composition used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of a medical practitioner.

An expression vector composition solution can include a therapeutically effective amount of a composition disclosed herein. Such effective amounts can be readily determined by one of ordinary skill in the art based, in part, on the effect of the administered composition, or the combinatorial effect of the composition and one or more additional active agents, if more than one agent is used. A therapeutically effective amount can be an amount at which any toxic or detrimental effects of the composition are outweighed by therapeutically beneficial effects.

In various embodiments, expression vector composition of the present disclosure could be used to deliver nucleotides to one or more tissues or cell types selected from liver, retina, skeletal muscle, and neurons or the central nervous system. In various embodiments, an expression vector composition can include 10² copies or more (e.g., at least 10², 10⁴, 10⁶, 10¹, 10¹⁰, 10¹², 10¹⁴, or 10¹⁶ copies) of a desired transgene (e.g., of a coding sequence operably linked with an S/MAR in an expression vector of the present disclosure).

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser may also be used, for example, with an injection system as disclosed herein.

In some embodiments, an expression vector composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions disclosed herein are stable in storage for at least 1 year at 2-8° C. (e.g., 4° C.). In particular instances, an expression vector composition can be formulated as a solution. In some embodiments, a composition can be formulated, for example, as a buffered solution at a suitable concentration and suitable for storage at 2-8° C. (e.g., 4° C.). In some embodiments, an expression vector composition can be formulated for storage at room temperature (20-25 degree Celsius).

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are known in the art.

### Applications

Various embodiments of the present disclosure further include administering an expression vector composition as disclosed herein to a cell, tissue, system, or a subject. Administration to the cell, tissue, system, or subject can be in vitro, ex vivo, or in vivo. Those of skill in the art will appreciate that an expression vector as disclosed herein can be used for diverse purposes, at least in part because diverse coding sequences can be operably linked with the S/MAR of the expression vector. Thus, at least one application of expression vectors and expression vector compositions disclosed herein is as a platform (e.g., as a therapeutic or laboratory tool) that may be used by those of skill in the art for expression of a coding sequence of interest. Those of skill in the art will therefore appreciate that various advantages of the present disclosure do not require or depend on identification or inclusion of any particular coding sequence operably linked with an S/MAR of an expression vector disclosed herein. In certain embodiments, an expression vector of the present disclosure can be used to achieve expression (e.g., a particular, improved, or target level, duration, and/or tissue specificity of expression) of a coding sequence of interest in one or more tissues or cell types, e.g., selected from liver, retina, skeletal muscle, and neurons or the central nervous system.

In various embodiments of the present disclosure, expression of a coding sequence operably linked with an S/MAR can be determined by measuring the number, concentration, proportion, or amount of mRNA transcripts corresponding to the coding sequence in a sample, cell, tissue, system, or subject. Various methods of measuring mRNA are well known in the art and include without limitation RNA-seq and reverse transcription PCR (e.g., quantitative reverse transcription PCR). In various embodiments qualitative or quantitative measurement of mRNA can be identified, e.g., as “increased” or “decreased” as compared to a reference.

In various embodiments of the present disclosure, expression of a coding sequence operably linked with an S/MAR can be determined by measuring the number, concentration, proportion, or amount of polypeptides corresponding to the coding sequence in a sample, cell, tissue, system, or subject. Various methods of measuring polypeptides are well known in the art and include without limitation mass spectrometry and immunohistochemistry. In various embodiments qualitative or quantitative measurement of polypeptide can be identified, e.g., as “increased” or “decreased” as compared to a reference.

In the event that a coding sequence is a representative coding sequence having reporter characteristics (e.g., fluorescence, luminescence, or other detectable characteristics), expression can be determined by detection of the reporter characteristic. The present disclosure includes the use of reporters including green fluorescent protein (GFP) and luciferase as representative coding sequences. Techniques for detecting and measuring such reporter characteristics are well known in the art. In various embodiments qualitative or quantitative measurement of a reporter characteristic can be identified, e.g., as “increased” or “decreased” as compared to a reference.

In various embodiments of the present disclosure, duration of expression of a coding sequence operably linked with an S/MAR can be determined by measuring expression over time. In various embodiments measurement of duration of expression can be identified, e.g., as “increased” or “decreased” as compared to a reference. In various embodiments, duration refers to a period between one of (i) administration, (ii) first detection of expression in an initial sample, cell, tissue, system, or subject, or (iii) the time at which expression reaches or exceeds a predetermined threshold level in an initial sample, cell, tissue, system, or subject, and one of (i) a time at which expression cannot be detected in a sample, cell, tissue, system, or subject comparable to the initial sample, cell, tissue, system, or subject, (ii) the time at which expression falls below a predetermined threshold level in a sample, cell, tissue, system, or subject comparable to the initial sample, cell, tissue, system, or subject. In various embodiments, duration of expression can be at least 5 days, 10 days, 2 weeks, 3 weeks, 1 month, 2 months, 3 months, 4 months, 5 months, 6 months, 9 months, 1 year, or more. In various embodiments, duration of expression can be within a range having a lower bound selected from 5 days, 10 days, 2 weeks, 3 weeks, or 1 month and an upper bound selected from, 2 weeks, 3 weeks, 1 month, 2 months, 3 months, 4 months, 5 months, 6 months, 9 months, 1 year, or more.

In various embodiments of the present disclosure, tissue specificity of expression and/or preferential expression can be determined by measuring the expression of a coding sequence in a first sample, cell, tissue, system, or subject of a first type and comparing it to a measurement of expression of the coding sequence in a second sample, cell, tissue, system, or subject of a second, different type, wherein a difference in expression between the measurements of expression demonstrates tissue specificity and/or preferential expression. In various embodiments issue specificity and/or preferential expression can refer to a state in which expression in a first sample, cell, tissue, system, or subject is at least 5% greater than in a second sample, cell, tissue, system, or subject, e.g., at least 5%, 10%, 15%, 20%, 25%, 50%, 75%, or 100% greater.

As used herein, tissue specificity of expression and/or preferential expression can also or alternatively be determined by measuring the duration of expression of a coding sequence in a first sample, cell, tissue, system, or subject of a first type and comparing it to a measurement of duration of expression of the coding sequence in a second sample, cell, tissue, system, or subject of a second, different type, wherein a difference in duration of expression between the measurements of duration of expression demonstrates tissue specificity and/or preferential expression. In various embodiments issue specificity and/or preferential expression can refer to a state in which duration of expression in a first sample, cell, tissue, system, or subject is at least 5% greater than in a second sample, cell, tissue, system, or subject, e.g., at least 5%, 10%, 15%, 20%, 25%, 50%, 75%, or 100% greater.

Notwithstanding the above, those of skill in the art will appreciate from the present disclosure that tissue specificity of expression is only one of several advantages associated with certain compositions and methods provided herein. Compositions and methods of the present disclosure do not necessarily demonstrate tissue specificity, and tissue specificity is not required for compositions and methods provided herein to demonstrate advantageous properties. For the avoidance of doubt, expression of a coding sequence in any of one or more tissues can be advantageous even in the absence of a demonstration of tissue specificity.

In various embodiments, the terms “increased,” “decreased,” and similar terms can be determined by comparing individual measurements or by comparing the mean, median, or mode of multiple measurements. In various embodiments, compared values can be designated as comparable if a non-reference value is within 50% of a reference value (e.g., within 40%, 30%, 25%, 20%, 15%, 10%, or 5%).

In certain embodiments of the present disclosure, comparison of expression or duration of expression can be between a first expression vector that includes an S/MAR of the present disclosure operably linked with a coding sequence and a second expression vector that is substantially the same as the first expression vector except in that it includes a reference S/MAR in place of the S/MAR of the first expression vector, optionally wherein the reference S/MAR is S/MAR huIFNb. In various embodiments, comparison is made between expression or duration of expression from expression vectors tested in samples, cells, tissues, systems, or subjects that are comparable (e.g., identical or substantially identical), e.g., the same type of sample, cell tissue, or system. Those of skill in the art will appreciate that results comparing expression or duration of expression in vitro, ex vivo, or in animal models is representative of expression or duration of expression in therapeutic contexts and applications.

In various embodiments, expression or duration of expression of a coding sequence operably linked with an S/MAR of the present disclosure is increased as compared to expression or duration of expression achieved by operable linkage of the coding sequence with a reference S/MAR under comparable and/or reference conditions. Expression or duration of expression of a coding sequence operably linked with an S/MAR of the present disclosure can be, e.g., increased as compared to a reference by at least 1%, 2%, 3%, 4%, 6%, 10%, 20%, 30%, 40%, 50%, or more.

In various embodiments, expression of a coding sequence operably linked with an S/MAR of the present disclosure is comparable with expression achieved by operable linkage of the coding sequence with a reference S/MAR under comparable and/or reference conditions. ression or duration of expression of a coding sequence operably linked with an S/MAR of the present disclosure can be, e.g., comparable in that it is increased or decreased as compared to a reference but is within 50% of a reference value (e.g., within 40%, 30%, 25%, 20%, 15%, 10%, or 5%).

The present disclosure includes a method of producing an expression vector of the present disclosure where the expression vector includes a coding sequence operably linked with an S/MAR. In various embodiments a method of producing the expression vector includes de novo synthesis of the expression vector or a portion thereof including the coding sequence operably linked with the S/MAR. In various embodiments a method of producing the expression vector in which an S/MAR is operably linked with a coding sequence includes insertion of a coding sequence into an expression vector that includes an S/MAR and a restriction site. Certain such methods can include steps of (i) contacting an expression vector including an S/MAR and a restriction site positioned for operative linkage with a restriction enzyme corresponding to the restriction site, thereby cleaving the restriction site and producing cleaved ends (in some embodiments, “sticky ends”), (ii) contacting the cleaved expression vector with a nucleic acid that includes a coding sequence and has termini for ligation with the cleaved ends, and (iii) ligating the coding sequence nucleic acid into the cleaved restriction site of the expression vector.

The present disclosure includes methods in which an expression vector of the present disclosure is administered to a cell, tissue, system, or subject, e.g., to achieve expression of a coding sequence operably linked with an S/MAR in the cell, tissue, system, or subject. The present disclosure further includes cells, tissues, systems, and subjects comprising expression vectors of the present disclosure, optionally where the expression vector is expressing the coding sequence operably linked with the S/Mar of the expression vector. Without limiting the general applicability of the expression vector technology disclosed herein, certain exemplary applications are provided below.

### Gene Therapy

In various embodiments, expression vectors provided herein can be used in methods of gene therapy. The general principles of gene therapy are well known in the art and include the delivery of a polynucleotide to a subject in need thereof to provide an expression product (e.g., an mRNA, protein, or inhibitory RNA) of therapeutic value. In some embodiments, gene therapy can include gene or protein replacement therapy (e.g., enzyme replacement therapy), augmentation, or target inhibition. In various embodiments, expression vectors provided herein can be applied to rescue deleterious effects of any mutations that cause diseases including, without limitation, Cystic fibrosis, Duchenne muscular dystrophy, Stargardt's disease, Age-related macular degeneration, Huntington, Hemophilia A, Spinal muscular atrophy, Usher syndrome etc. In such diseases, a genetic mutation renders a gene nonfunctional or not available. In such cases, replacing the mutated gene by a functional copy may be beneficial to patients.

In some embodiments, expression vectors provided herein can be applied to inhibit genes that are upregulated and disease causing. For example, P53 overexpression has been described in various diseases. In some instances, it is also beneficial to knock down genes at specific cells or tissues to downregulate genes that cause inflammation, hypoxia, etc. to achieve a therapeutic benefit. Expression vectors of the present disclosure can also be useful for various cosmetic applications for example to enhance muscle mass, repair skin in burn victims, cause weight loss, improve immune function, or slow aging.

By operably linking an S/MAR with a coding sequence in an expression vector, and delivering the expression to cells or tissues of a subject, in various embodiments, a therapeutic or cosmetic benefit can result. In various embodiments, an expression vector for gene therapy is associated with a mini-nucleosome core protein

### Ex Vivo Engineered Cells

Expression vectors of the present disclosure can be used to engineer cells ex vivo. Cells can be engineered to express therapeutic expression products by delivery of an expression vector to the cells. In various embodiments, expression vectors are delivered to immune cells, e.g., T cells. Immune cells can be genetically engineered to express new proteins or receptors that may allow immune recognition of cancerous cells or other harmful cell types for killing and clearance. Such genetic engineering may be performed ex vivo. An expression vector can encode and/or express products such as chimeric receptors, inhibitory RNAs for knockdown of genes, or other therapeutic expression products. In various embodiments, expression vectors can be used to produce chimeric antigen receptor T cells (CAR T cells) for use in immunotherapy. In some embodiments, expression vectors provided herein can be applied to engineering stem cells ex vivo to express new proteins or receptors for therapeutic purposes. In various embodiments, expression vectors can be used to produce chimeric stem cells such as chimeric hematopoietic stem cells for use in immunotherapy. In various embodiments, engineered cells can then be infused into patients for therapeutic benefit. In various embodiments, an expression vector for ex vivo modification of cells is associated with a mini-nucleosome core protein

### Gene Editing and Base Excision Repair

Gene editing, such as base editing and other manipulations, can also be achieved using expression vectors that encode expression products for such purposes. Gene editing technologies and base excision can be used in correcting a genetic mutation or editing endogenous genes of target cells at the DNA or RNA level. Accordingly, an expression vector may encode and/or express, optionally in operable linkage with an S/MAR, one or more of a gRNA, sgRNA, spCas9, saCas9, dCas9, cytidine deaminase, or any of various other enzymes of gene editing systems known in the art, which can in various embodiments cleave DNA or convert one base to another. A plurality of gRNAs and Cas9 or similar editing enzymes can be delivered to target cells in a single vector or in multiple vectors. Providing a plurality of gRNAs and Cas9 can be cumbersome, inefficient, or otherwise challenging when attempted using certain other systems, such as an AAV. In various embodiments, an expression vector for gene editing is associated with a mini-nucleosome core protein

### Antibody Delivery

Antibodies are a class of drugs that have been life changing for millions of patients worldwide. However, many treatments based on delivery of antibodies to subjects have the drawback of requiring repeat administration, which poses immense burden to patients, physicians, and caregivers. Expression vectors disclosed herein can encode and/or express an antibody. Such expression vectors can therefore be used to vectorize antibodies and deliver the antibody, via the expression vector, to desired cells in the patients. In various embodiments, administration of an expression vector in which a coding sequence encoding an antibody is operably linked with an S/MAR can produce a depot, e.g., a long-term depot, in subjects bodies and/or reduce the need and/or burden of repeat administration (e.g., reducing the number or frequency of repeat administration). In various embodiments, a vectorized antibody can be, for example, a nanobody, antibody mimetic, fusion peptide, antibody fragment, or camelid or camelid single-domain antibody fragment. In various embodiments, an expression vector for antibody delivery is associated with a mini-nucleosome core protein

### Vaccine Delivery

In various embodiments, an expression vector disclosed herein can encode and/or express an antigen. Delivery of an expression vector that encodes and/or expresses an antigen to subject can induce a protective immunological response. Vaccines based upon delivery to a subject of a nucleic acid encoding an antigen can be referred to as nucleic acid vaccines. Nucleic acid vaccines can have several advantages over certain types of conventional vaccines, including without limitation induction of a wide-range immunological response. Expression vectors that encode and/or express an antigen can be delivered to subjects, cells, or tissues to provide protection against, e.g., viral, bacterial or parasitic infections. In various embodiments, an expression vector for vaccine delivery is associated with a mini-nucleosome core protein

### EXAMPLES

The present Examples demonstrate identification and validation of S/MARs particularly useful for controlling expression of operably linked coding sequences (e.g., to achieve a particular, improved, or target level, duration, and/or tissue specificity of expression) in one or more tissues or cell types selected from liver, retina, skeletal muscle, and neurons or the central nervous system. Although putative S/MARs have been identified, few have been characterized for characteristics related to controlling expression of operably linked coding sequences in particular tissues or cell types. The present Examples therefore confirm that S/MARs of the present disclosure can be used to achieve advantageous expression of expression products in target tissues or cell types, e.g., for gene therapy.

Various Examples provided herein include known S/MAR huIFNb as a positive control. huIFNb is a well-known S/MAR recognized by those of skill in the art as mediating robust expression of operably linked coding sequences. S/MAR huIFNb can mediate expression of operably linked transgenes, e.g., in Liver, eye, muscle and CNS.

Surprisingly, the present disclosure identifies S/MARs that mediate expression of operably linked coding sequences at a level that is comparably to or greater than the expression achieved by huIFNb. For example, the S/MAR identified herein as SUM1 conferred expression of an operably linked representative coding sequence at a level greater than that achieved using huIFNb across all tested cell types, in particular in both HEK 293T and the neuronal cell line Neuro2A. Moreover, S/MARs identified herein as SUM2 and SUM3 conferred expression of operably linked coding sequences at a level comparable to that of huIFNb, and moreover demonstrated specificity of expression in Neuro 2A cells. SUM2 and SUM3 each demonstrated greater specifity for neuronal expression than SUM1 or huIFNb. These results are representative, not exhaustive, of the various advantages provided by S/MARs of the present disclosure and expression vectors including the same. S/MARs of the present disclosure and expression vectors including the same provide valuable tools for achieving expression of coding sequences at particular, improved, or target level, duration, and/or tissue specificity of expression), e.g., in one or more tissues or cell types.

The present Examples further demonstrate that expression vectors of the present disclosure achieve enduring expression following delivery of the expression vector. In various exemplified instances, following an initial decrease in total expression or expressing cells, expression of a coding sequence operably linked with an S/MAR was shown to demonstrate long-term durability. To provide on particular example, following an expected initial decrease in the number of GFP expressing cells, SUM1 mediated stable and enduring expression of a representative coding sequence in HEK 293T cells over the time at levels similar to those achieved by a huIFNb reference S/MAR. SUM2 similarly achieved enduring expression comparable to that achieved using a huIFNb reference S/MAR or SUM1 S/MAR.

### Example 1: Identification of S/MARs for Expression in Target Tissues or Cell Types

The present Example provides the identification of S/MARs useful for controlling expression of operably linked coding sequences (e.g., to achieve a particular, improved, or target level, duration, and/or tissue specificity of expression) in one or more tissues or cell types selected from liver, retina, skeletal muscle, and neurons or the central nervous system.

One step in identifying tissue-specific S/MARs was to correlate control of expression of operably linked coding sequences by particular S/MARs with particular tissues or cell types. The present Example includes the present inventor's development of in-silico approach to select S/MAR sequences of the human genome (human hg38 genome). To determine expression levels of coding sequences associated with S/MARs across tissues, expression of the first coding sequence downstream of each S/MAR of interest was evaluated across 37 tissues using RNA-seq (transcript expression) data. S/MARs reproducibly associated with higher transcription of operably linked coding sequences in one or more tissues or cell types selected from liver, retina, skeletal muscle, and neurons or the central nervous system as compared to other tissues or cell types were selected for further analysis.

To further examine the tissue specificity of control of expression of operably linked sequences by various S/MARs, the present inventors analyzed the expression of certain S/MARbps (BRIGHT, CEBPB, CTCF, FOXH1, Ku autoantigen, NMP4, Mut p53, hnRNPU, SATB1, YY1) across tissues using RNA-seq (transcript expression) data. S/MAR sequences including S/MARbp binding sites of S/MARbps expressed in the same cell types or tissues as the those in which the S/MAR controlled expression were selected for further analysis.

Various other criteria were also applied. In the present Example, S/MARs were selected that were characterized by the presence of all seven of the S/MAR features including (i) an origin of replication (OriC) sequence, (ii) AT richness, (iii) kinked DNA, (iv) curved DNA, (v) TG richness, (vi) MAR signature, and (vii) a Topoisomerase-II site. S/MAR sequences over 2 kb in length were excluded, as sequence length can be a limiting factor for inclusion in various size-limited vectors. S/MAR sequences positioned with introns of coding sequences were also excluded, as S/MARs positioned within genes (e.g., within introns) have generally been found to have weaker interactions with S/MARbps than other S/MARs (e.g., as determined by affinity for nuclear matrix in the presence of an excess of nonspecific DNA). S/MAR sequences more proximal to transcription start sites were preferred.

Ten S/MARs were selected for use in expression constructs in accordance with the criteria disclosed in the present example and which cause high expression of operably linked sequences in a tissue or cell type selected from liver, retina, skeletal muscle, and neurons or the central nervous system. The sequences of these S/MARs are disclosed in Table 4 and correspond to SEQ ID NOs: 1-10.

### Example 2: In Vitro Demonstration of S/MAR Control of Expression

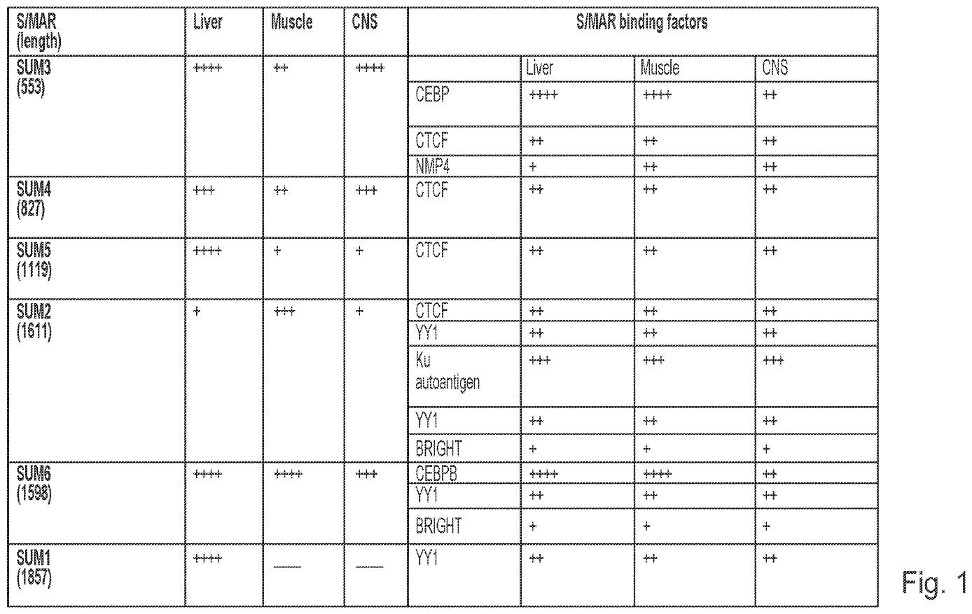

In the present Example, six S/MARs found to cause expression in one or more of liver, retina, skeletal muscle, and neurons or the central nervous system, and to bind one or more S/MARbps expressed in liver, retina, skeletal muscle, and neurons or the central nervous system, were further analyzed for control of expression in selected cell types (FIG. 1). Expression of a coding sequence operably linked with an S/MAR in one or more of the cell types confirms that expression constructs including the S/MAR can be beneficially used in gene therapy.

In the present Example, S/MARs were positioned 3′ of, and thereby operably linked with, a luciferase reporter and expressed in HEK 293T cells (ATCC® CRL-3216™) and neuronal cell line Neuro2A cells (ATCC, CCL-131™). Expression of the reporter encoded by a coding sequence operably linked with SMAR's selected in the present Examples (SEQ ID NOs: 1-6) were compared to expression of a reporter encoded by a coding sequence operably linked with a reference S/MAR, hu1FNb.

In these experiments, cells were cultured in EMEM (ATCC, 30-2003) supplemented with 10% fetal bovine serum (ATCC, 30-2020) and 100 U/ml penicillin-streptomycin (GIBCO, 15140-122) at 37° C. and 5% CO2 on 96 Well White/Clear Bottom Plate (165306, Thermo Scientific). Cells were transfected with the plasmid encoding the luciferase reporter and S/MAR sequences of huIFNb, SUM1, SUM2, SUM3, SUM4, SUM5, and SUM6 located 3′ of the reporter. Cells were transfected using Lipofectamine LTX and Plus Reagent per manufacturer's instructions (Invitrogen, 15338-100). Transfected cells were cultured for 48 h before detection of firefly luciferase reporter gene expression by following the manufacture protocol of ONE-Glo™ Luciferase Assay System (E6110, Promega). The luminescence signal was measured as a direct variable of luciferase activity indication in the plate reader GLOMAX Discover microplate reader (GM3000, Promega).

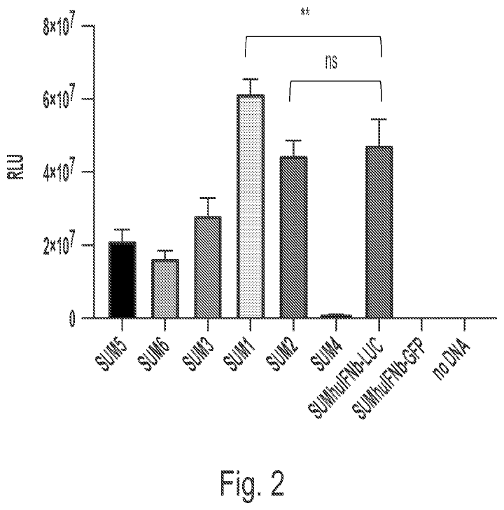

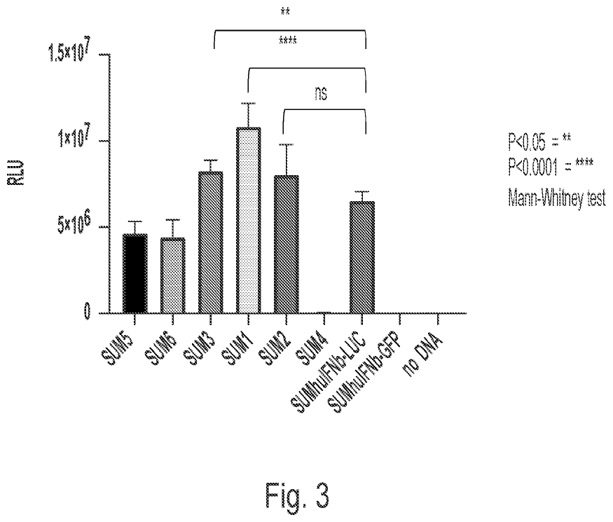

These experiments provide data relating to the stability of exogenous nucleic acids including S/MARs in cells and the expression of coding sequences operably linked with the S/MARs. Data shown in FIGS. 2 and 3 (from HEK 293T and Neuro2A cells respectively) demonstrate that expression from plasmids in which the luciferase coding sequence is operably linked with SUM1 or SUM3 is higher than the control plasmid in which the luciferase coding sequence is operably linked with hu1FNb S/MAR in both tested cell types. Statistical significance of the increase in expression as compared to the reference was greatest in the Neuro 2A cells for SUM1, and similar between the two cell types for SUM3. SUM2, SUM5, and SUM6 also each successfully demonstrated expression of luciferase at levels comparable to the reference. Thus, expression demonstrated for SUM1, SUM2, SUM3, SUM5, and SUM6 demonstrated high levels of expression sufficient to confer advantageous expression of an operably linked coding sequence, e.g., for use in gene therapy. As those of skill in the art will appreciate, such expression indicates tested S/MARs have expression-regulatory characteristics including causing or contributing to expression (e.g., tissue specific expression) of operably linked coding sequences in tissues or cell types selected from liver, retina, skeletal muscle, and neurons or the central nervous system in accordance with expression driven by hu1FNb in such same tissues.

By contrast, SUM4 did not cause expression of an operably linked coding sequence.

### Example 3: In Vitro Demonstration of S/MAR Control of Expression

The present Example is similar to Example 2 but utilizes a green fluorescence protein (GFP) reporter. S/MARs were positioned 3′ of, and thereby operably linked with, a GFP reporter and expressed in HEK 293T cells (ATCC® CRL-3216™). Expression of the reporter encoded by a coding sequence operably linked with SMAR's selected in the present Examples (SEQ ID NOs: 1-3) were compared to expression of a reporter encoded by a coding sequence operably linked with a reference S/MAR, hu1FNb.

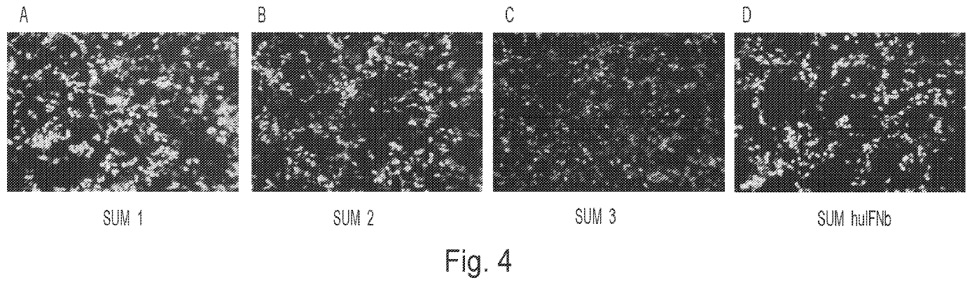

In these experiments, cells were cultured in EMEM (ATCC, 30-2003) supplemented with 10% fetal bovine serum (ATCC, 30-2020) and 100 U/ml penicillin-streptomycin (GIBCO, 15140-122) at 37° C. and 5% CO2 on tissue culture plates 24 wells (734-2325, VWR). Cells were transfected with the plasmid encoding the GFP reporter and S/MAR sequences of huIFNb, SUM1, SUM2 and SUM3 located 3′ of the reporter (phuIFNb, pSUM1, pSUM2, pSUM3). Cells were transfected using Lipofectamine LTX and Plus Reagent per manufacturer's instructions (Invitrogen, 15338-100). Transfected cells were cultured for 48 h before imaging under a 20× plan fluorite objective from an EVOS FLoid Imaging System (4471136, Thermo Fisher Scientific). Representative images of the reporter expression 48 h post-transfection are shown in FIG. 4, panels A-D (10× objective, GFP signal in green color). Data demonstrate that SUM1, SUM2, and SUM3 each achieve successful expression similar to or greater than expression associated with hu1FNb in accordance with other Examples of the present disclosure.

### Example 4: In Vitro Demonstration of Long Term Expression of Coding Sequences Operably Linked with S/MARs

In the present Example, three S/MARs of the present disclosure were further analyzed to determine the stability of expression of coding sequences operably linked with S/MARs over long periods of time. S/MARs SUM1, SUM2, and SUM3, or reference S/MAR hu1FNb, were positioned 3′ of, and thereby operably linked with, a GFP reporter in plasmids designated pSUM1, pSUM2, pSUM3, and phuIFNb.

In these experiments, 293T cells were plated in T75 tissue culture flask (353136, Falcon) and transfected with each of the plasmids (pSUM1, pSUM2, pSUM3, phuIFNb) using Lipofectamine LTX and Plus Reagent per manufacturer's instructions (Invitrogen, 15338-100). 72h post-transfection, cells were trypsinized and resuspended in sorting media composed of DPBS 1× without calcium and magnesium (21-031-CV, Corning), 2% fetal bovine serum (ATCC, 30-2020) and the viability marker 7AAD (R37173, Invitrogen). The GFP expressing cells of each transfection were sorted in a FACSAria-III Cell sorter/application FACSDiva Version 8.0.2 (Morphology Dept., School of Medicine, UMASS; Sorting efficiency: SUM1, 80%; SUM2, 76%; SUM3, 72%; SUMhuIFNb, 70%). Non-transfected cells were used as a control reference for setting sorting parameters before running the transfected samples. The sorting cells were collected in a media of EMEM (ATCC, 30-2003) supplemented with 20% fetal bovine serum (ATCC, 30-2020), 25 mM HEPES pH 7 (15630-080, Gibco) and 100 U/ml penicillin-streptomycin (GIBCO, 15140-122). The collected GFP expressing cells of each transfection were spun down at 1200 rpm/5 min at room temperature followed by resuspension in culture media (EMEM (ATCC, 30-2003) supplemented with 10% fetal bovine serum (ATCC, 30-2020) and 100 U/ml penicillin-streptomycin (GIBCO, 15140-122) at 37° C. and 5% C02). The cells were plated in tissue culture plates 24 wells (734-2325, VWR) as 300,000 cells/wells and they were cultured from a total period of 28 days, corresponding to ten cell passages. Triplicate wells, per each transfection, were imaged before each passage to calculate the total GFP expressing cell numbers under the transcriptional control of each S/MAR sequence. Imaging and quantification were by a Cytation 5 Cell Imaging Multi-Mode Reader/Gen5 software (BioTek) with 4× objective and auto exposure mode.

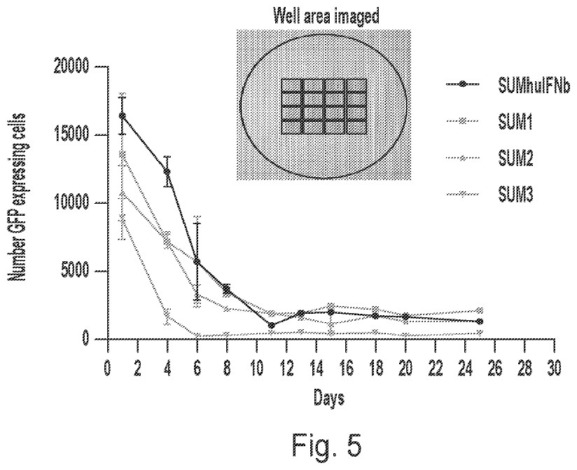

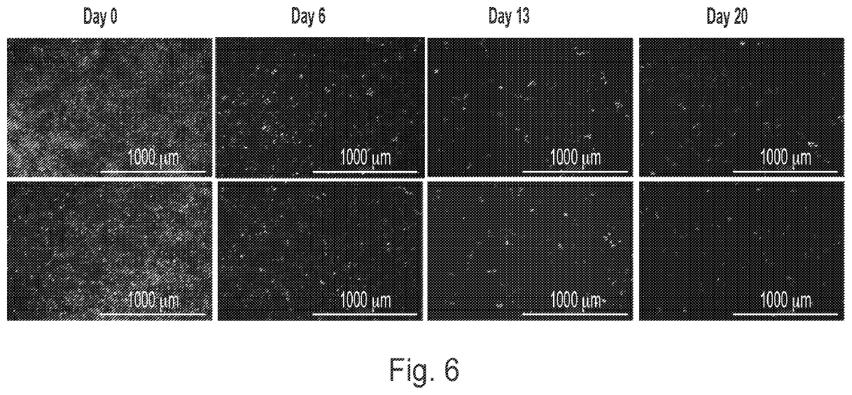

FIG. 5 shows the total number of GFP expressing cells at each time point corresponding to the average of 3 replicates imaged areas of M24 wells before each passage. FIG. 6 shows representative images of SUMhuIFNb (upper panels) and SUM1 (lower panels) expressing cells for the indicated time points. Data show that duration of expression of a coding sequence operably linked with SUM1 is similar to expression of a coding sequence operably linked with the reference S/MAR, with similar results achieved using SUM2. Moreover, duration of expression of a coding sequence operably linked with SUM2 demonstrated expression sufficient to confer advantageous expression of an operably linked coding sequence, e.g., for use in gene therapy. SUM3 was not found to provide the duration of expression observed for SUM1 and SUM2 in HEK293T cells, consistent with its specificity for neuronal cells. Data demonstrate that SUM1, SUM2, and SUM3 each achieve successful expression similar to or greater than expression associated with hu1FNb in accordance with other Examples of the present disclosure.

### Example 5: In Vitro Demonstration of S/MAR Control of Expression

The present Example demonstrates and confirms that S/MARs of the present disclosure cause and/or increase expression of operably linked coding sequences. In the present Example, five S/MARs were operably linked with luciferase and expressed in C2C12 mouse myoblasts.

In these experiments, cells were cultured in DMEM (ATCC, 30-2002) supplemented with 10% fetal bovine serum (ATCC, 30-2020) and 100 U/ml penicillin-streptomycin (GIBCO, 15140-122) at 37° C. and 5% C02 on 96 Well White/Clear Bottom Plate (165306, Thermo Scientific). Cells were transfected with the plasmid encoding the luciferase reporter and S/MAR sequences of SUM1, SUM2, SUM3, SUM5 and SUM6 located 3′ of the reporter or a plasmid with a reference S/MAR, hu1FNb (SUMhu1FNb). Cells were transfected using Lipofectamine LTX and Plus Reagent per manufacturer's instructions (Invitrogen, 15338-100). Transfected cells were cultured for 48 h before detection of firefly luciferase reporter gene expression by following the manufacture protocol of ONE-Glo™ Luciferase Assay System (E6110, Promega). The luminescence signal was measured as a direct variable of luciferase activity indication in the plate reader GLOMAX Discover microplate reader (GM3000, Promega).

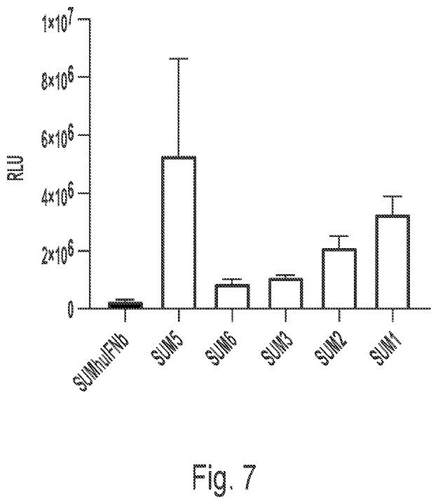

As shown in FIG. 7, plasmids encoding S/MARs (SUM1, SUM2, SUM3, SUM5 and SUM6) respectively) or a plasmid with a reference S/MAR, hu1FNb (SUMhu1FNb) were expressed in C2C12 mouse myoblasts. Constructs including these regulatory sequences operatively linked with a nucleic acid sequence encoding luciferase are referred to in FIG. 7 as SUMhu1FNb, SUM1, SUM2, SUM3, SUM5 and SUM6. Results demonstrate that each of the S/MARs drove expression, e.g., at a greater level than the representative plasmid with the reference S/MAR hu1FNb.

### Example 6: In Vivo Confirmation of S/MAR Control of Expression

The present Example demonstrates and confirms that S/MARs of the present disclosure cause and/or increase expression of operably linked coding sequences in vivo. In the present Example, three S/MARs were operably linked with luciferase and expressed in mice.

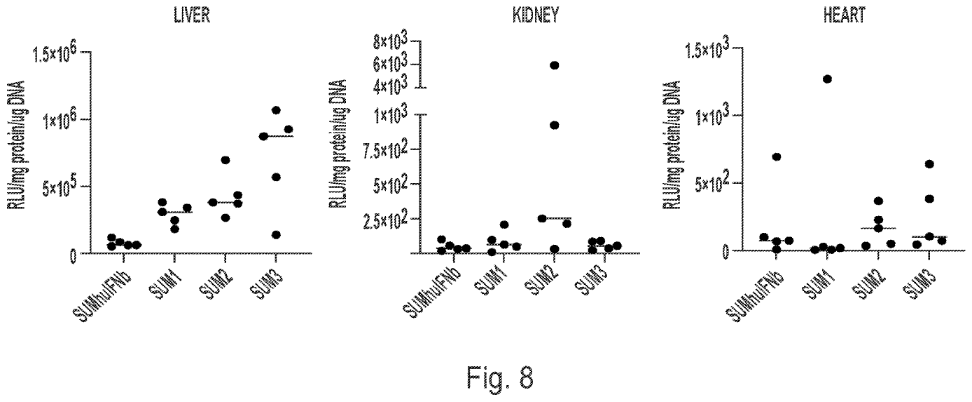

As shown in FIG. 8, plasmids encoding S/MARs (SUM1, SUM2 or SUM3, respectively) or a reference S/MAR hu1FNb (SUMhu1FNb) were expressed in BALB/c mice after system administration by hydrodynamic intravenous injection. Constructs including these regulatory sequences operatively linked with a nucleic acid sequence encoding luciferase are referred to in FIG. 8 as SUMhu1FNB, SUM1, SUM2 and SUM3, respectively. BALB/c mice were given a single hydrodynamic intravenous injection of 100 ug plasmid DNA (100 ml/kg) over approximately 5 seconds into the tail vein. Mice were euthanized 72 hours following injection and tissue was collected following cardiac perfusion with PBS. Liver, heart, and kidney tissues were separately homogenized in Glo-lysis buffer (Promega, E2661) using gentleMACS M-tubes (i Biotec).

ONE-Glo™ Luciferase Assay System substrate (Promega, E6610) was added to the tissue lysates and luciferase expression was measured using the GLOMAX Discover microplate reader (GM3000, Promega). Data is represented relative to total protein concentration of the tissue lysate and DNA concentration of injected plasmids (n=4-5/group). Data demonstrate that tested S/MARs cause and/or increase expression of operably linked coding sequences in vivo in mice, particularly in liver, heart, and kidney, e.g., at a greater level than the plasmid with a reference S/MAR hu1FNb.

### Example 7: Demonstration of S/MAR Control of Therapeutic Gene Expression In Vivo

The present Example demonstrates and confirms that S/MARs of the present disclosure cause and/or increase expression of operably linked therapeutic coding sequences in vivo. In the present Example, a representative S/MAR (SUM3) was operably linked with a nucleic acid sequence encoding a representative protein therapeutic, aflibercept (Eylea) and expressed in mice following system administration. Aflibercept is a protein therapeutic used to treat certain conditions of the eye including age-related macular degeneration (AMD, e.g., Wet AMD), diabetic eye disease, and retinal diseases.

In this example, a plasmid including a nucleic acid sequence encoding aflibercept, which nucleic acid sequence was operably linked to S/MAR SUM3, was administered to C57B6 mice by hydrodynamic intravenous injection. C57B6 mice were given a single hydrodynamic intravenous injection of 10 ug plasmid DNA over approximately 5 seconds into the tail vein. Plasmids included the nucleic acid sequence encoding aflibercept, with or without an operatively linked S/MAR SUM3 (Reference-SUM3) or a reference S/MAR, Mice were euthanized 72 hours following injection and serum was collected. Aflibercept levels in serum were measured using an ELISA assay. Briefly, VEGF-165 (R&D systems; 293-VE-010/CF) was coated on a plate at a final concentration of 1 μg/ml, followed by incubation of supernatants and lysates. Eylea was detected using an HRP-labeled anti-IgG antibody. Recombinant Eylea (Aflibercept drug product) was used as a standard. (n=5/group for plasmids, n=2 for untreated samples). Data obtained demonstrates that operably linking an S/MAR to a nucleic acid sequence encoding a protein therapeutic controls and increases expression of the protein therapeutic compared to a reference S/MAR (data not shown).

### REFERENCES

O Argyros, S P Wong & R P Harbottle. Non-viral episomal modification of cells using S/MAR elements. Expert Opinion on Biological Therapy. 2011 May 9; 11:9, 1177-1191. doi: 10.1517/14712598.2011.582035.
O Argyros, S P Wong, M Niceta, S N Waddington, S J Howe, C Coutelle, A D Miller and R P Harbottle. Persistent episomal transgene expression in liver following delivery of a scaffold/matrix attachment region containing non-viral vecto. Gene Therapy. 2008, 15, 1593-1605.
C M van Drunen, R G Sewalt, R W Oosterling, P J Weisbeek, S C Smeekens, R van Driel. A bipartite sequence element associated with matrix/scaffold attachment regions. Nucleic Acids Res. 1999 Jul. 15; 27(14): 2924-2930. doi: 10.1093/nar/27.14.2924.
K Evans, S Ott, A Hansen, G Koentges, L Wernisch. A comparative study of S/MAR prediction tools. BMC Bioinformatics. 2007; 8: 71. Published online 2007 Mar. 2. doi: 10.1186/1471-2105-8-71.
A M Fletcher, T H Kowalczyk, L Padegimas, M J Cooper, D M Yurek. Transgene expression in the striatum following intracerebral injections of DNA nanoparticles encoding for human glial cell line-derived neurotrophic factor (hGDNF). Neuroscience. 2011 Oct. 27; 194: 220-226. doi: 10.1016/j.neuroscience.2011.07.072.
M Frisch, K Frech, A Klingenhoff, K Cartharius, I Liebich, T Werner. In Silico Prediction of Scaffold/Matrix Attachment Regions in Large Genomic Sequences. Genome Res. 2002 February; 12(2): 349-354. doi: 10.1101/gr.206602.
Harraghy N., Regamey A., Girod P.-A., Mermod N. Transient Expression Technologies: Past, Present, and Future. J. Biotechnol. 2011. V. 154. P. 11-20.
M D Kaytor, M R Weatherspoon, K J Green, T H Kowalczyk, L Padegimas, M J Cooper. In Vivo Delivery of Nucleic Acid to the Brain Using DNA Nanoparticles. Molecular Therapy. 2009, doi.org/10.1016/S1525-0016(16)38877-3.
A Koirala, R S Makkia, S M Conley, M J Cooper, M I Naash. S/MAR-containing DNA nanoparticles promote persistent RPE gene expression and improvement in RPE65-associated LCA. Hum Mol Genet. 2013 Apr. 15; 22(8): 1632-1642. Published online 2013 Jan. 18. doi: 10.1093/hmg/ddt013.
Y S Li, E Davidson, C N. Reid, A P McHale. Optimising ultrasound-mediated gene transfer (sonoporation) in vitro and prolonged expression of a transgene in vivo: Potential applications for gene therapy of cancer. Cancer Letters 273 (2009) 62-69.
N Narwade, S Patel, A Alam, S Chattopadhyay, S Mittal, A Kulkarni. Mapping of scaffold/matrix attachment regions in human genome: a data mining exercise. Nucleic Acids Res. 2019 Aug. 22; 47(14): 7247-7261. doi: 10.1093/nar/gkz562.
C Piechaczek, C Fetzer, A Baiker, J Bode, H J Lipps. A vector based on the SV40 origin of replication and chromosomal S/MARs replicates episomally in CHO cells. Nucleic Acids Res. 1999 Jan. 15; 27(2):426-8. doi: 10.1093/nar/27.2.426.
G B Singh, J A Kramer, S A Krawetz. Mathematical model to predict regions of chromatin attachment to the nuclear matrix. Nucleic Acids Res. 1997 Apr. 1; 25(7): 1419-1425. doi: 10.1093/nar/25.7.1419.
I M Stehle, J P, S Rupprecht, T Cremer, D A Jackson, H J Lipps. Establishment and mitotic stability of an extra-chromosomal mammalian replicon. BMC Cell Biol. 2007; 8: 33. Published online 2007 Aug. 6. doi: 10.1186/1471-2121-8-33.
S P Wong, O Argyros, C Coutelle, R P Harbottle. Strategies for the episomal modification of cells. Curr Opin Mol Ther 2009; 11:433-41.

### OTHER EMBODIMENTS

It will be appreciated that the scope of the present disclosure is to be defined by that which may be understood from the disclosure and claims rather than by the specific embodiments that have been presented by way of example. Elements described with respect to one aspect or embodiment of the present disclosure are also contemplated with respect to other aspects or embodiments of the present disclosure. For example, elements of claims that depend directly or indirectly from a certain independent claim presented herein serve as support for those elements being presented in additional dependent claims of one or more other independent claims. Throughout the description, where compositions or methods are described as having, including, or comprising specific elements, compositions that consist essentially of, consist of, or do not comprise the recited elements are likewise hereby disclosed. All references cited herein are hereby incorporated by reference.

What is claimed is:

1. An expression vector comprising an S/MAR, wherein the S/MAR has at least 80% sequence identity with one of SEQ ID NOs: 1-10.

2. The expression vector of claim 1, wherein the S/MAR has at least 80% sequence identity with one of SEQ ID NOs: 1-6.

3. An expression vector comprising an S/MAR, wherein the S/MAR has at least 80% sequence identity with an S/MAR of Table 3.

4. The expression vector of any one of claims 1-3, wherein the S/MAR comprises (i) an origin of replication (OriC) sequence, (ii) AT richness, (iii) kinked DNA, (iv) curved DNA, (v) TG richness, (vi) a MAR signature, (vii) a Topoisomerase-II site, or (viii) a combination thereof.

5. The expression vector of any one of claims 1-4, wherein the S/MAR comprises binding sites for (i) BRCA1, (ii) BRIGHT, (iii) SMAR1, (iv) CEBPB, (v) CUX1/CDP, (vi) CTCF, (vii) Fast1/FOXH1, (viii) HoxC11, (ix) Ku autoantigen, (x) NMP4, (xi) Mut-p53, (xii) SAF-A/hnRNPU, (xiii) SATB1, (xiv) YY1, or (xv) a combination thereof.

6. The expression vector of any one of claims 1-5, wherein the S/MAR comprises binding sites for (i) BRIGHT, (ii) CEBPB, (iii) CTCF, (iv) Fast1/FOXH1, (v) Ku autoantigen, (vi) NMP4, (vii) Mut p53, (viii) SAF-A/hnRNPU, (ix) SATB1, (x) YY1, or (xi) a combination thereof.

7. The expression vector of any one of claims 1-6, wherein the S/MAR is operably linked with a coding sequence.

8. The expression vector of claim 7, wherein the S/MAR is upstream of the coding sequence, optionally wherein the S/MAR is no more than 2 kb, 1 kb, 500 bp, or 100 bp upstream of a transcription start site of the coding sequence.

9. The expression vector of claim 7, wherein the S/MAR is downstream of the coding sequence, optionally wherein the S/MAR is no more than 2 kb, 1 kb, 500 bp, or 100 bp downstream of the coding sequence.

10. The expression vector of claim 7, wherein the S/MAR is present in an intron of the coding sequence.

11. The expression vector of any one of claims 1-10, wherein the expression vector is a circular nucleic acid, optionally wherein the expression vector is a plasmid.

12. The expression vector of any one of claims 1-10, wherein the expression vector is a linear nucleic acid.

13. The expression vector of any one of claims 1-12, wherein the expression vector is associated with an engineered polypeptide comprising (i) a nucleic acid binding domain and (ii) a nucleic acid release domain, a nuclear localization signal, a stability domain, an oligomerization domain, a targeting domain, or a combination thereof, optionally wherein the engineered polypeptide is a mini-nucleosome core protein.

14. The expression vector of any one of claims 1-13, wherein the expression vector is a viral genome and/or is present in a viral vector.

15. The expression vector of any one of claims 1-13, wherein the expression vector is an engineered expression vector.

16. A method of expressing a coding sequence in liver cells, retinal cells, skeletal muscle cells, neuronal cells, central nervous system cells, or a combination thereof, the method comprising delivering an expression vector to the liver cells, retinal cells, skeletal muscle cells, neuronal cells, central nervous system cells, or combination thereof,
wherein the expression vector comprising an S/MAR operably linked with a coding sequence,
wherein the S/MAR has at least 80% sequence identity with one of SEQ ID NOs: 1-10,
optionally wherein the delivering comprises administering the expression vector to a subject, and
optionally wherein the subject is a human.

17. The method of claim 16, wherein the S/MAR has at least 80% sequence identity with one of SEQ ID NOs: 1-6.

18. A method of expressing a coding sequence in liver cells, retinal cells, skeletal muscle cells, neuronal cells, central nervous system cells, or a combination thereof, the method comprising delivering an expression vector to the liver cells, retinal cells, skeletal muscle cells, neuronal cells, central nervous system cells, or combination thereof,
wherein the expression vector comprising an S/MAR operably linked with a coding sequence,
wherein the S/MAR has at least 80% sequence identity with an S/MAR of Table 3,
optionally wherein the delivering comprises administering the expression vector to a subject, and
optionally wherein the subject is a human.

19. The method of any one of claims 16-18, wherein expression of the coding sequence in the liver cells, retinal cells, skeletal muscle cells, neuronal cells, central nervous system cells, or combination thereof is characterized by (i) increased expression as compared to a reference, (ii) increased duration of expression as compared to a reference, (iii) comparable expression as compared to a reference, (iv) comparable duration of expression as compared to a reference, or (v) a combination thereof.

20. The method vector of any one of claims 16-19, wherein the expression vector is associated with an engineered polypeptide comprising (i) a nucleic acid binding domain and (ii) a nucleic acid release domain, a nuclear localization signal, a stability domain, an oligomerization domain, a targeting domain, or a combination thereof, optionally wherein the engineered polypeptide is a mini-nucleosome core protein.

21. The method vector of any one of claims 16-20, wherein the expression vector is a viral genome and/or is present in a viral vector.

22. A method of producing an expression vector composition comprising contacting (i) an expression vector comprising an S/MAR operably linked with a coding sequence, wherein the S/MAR has at least 80% sequence identity with one of SEQ ID NOs: 1-10, with (ii) an engineered polypeptide comprising (a) a nucleic acid binding domain and (b) a nucleic acid release domain, a nuclear localization signal, a stability domain, an oligomerization domain, a targeting domain, or a combination thereof, optionally wherein the engineered polypeptide is a mini-nucleosome core protein.

23. The method of claim 22, wherein the S/MAR has at least 80% sequence identity with one of SEQ ID NOs: 1-6.

24. A method of producing an expression vector composition comprising contacting (i) an expression vector comprising an S/MAR operably linked with a coding sequence, wherein the S/MAR has at least 80% sequence identity with an S/MAR of Table 3, with (ii) an engineered polypeptide comprising (a) a nucleic acid binding domain and (b) a nucleic acid release domain, a nuclear localization signal, a stability domain, an oligomerization domain, a targeting domain, or a combination thereof, optionally wherein the engineered polypeptide is a mini-nucleosome core protein.

25. The method of any one of claims 16-24, wherein the S/MAR comprises (i) an origin of replication (OriC) sequence, (ii) AT richness, (iii) kinked DNA, (iv) curved DNA, (v) TG richness, (vi) a MAR signature, (vii) a Topoisomerase-II site, or (viii) a combination thereof.

26. The method of any one of claims 16-25, wherein the S/MAR comprises binding sites for (i) BRCA1, (ii) BRIGHT, (iii) SMAR1, (iv) CEBPB, (v) CUX1/CDP, (vi) CTCF, (vii) Fast1/FOXH1, (viii) HoxC11, (ix) Ku autoantigen, (x) NMP4, (xi) Mut-p53, (xii) SAF-A/hnRNPU, (xiii) SATB1, (xiv) YY1, or (xv) a combination thereof.

27. The method of any one of claims 16-26, wherein the S/MAR comprises binding sites for (i) BRIGHT, (ii) CEBPB, (iii) CTCF, (iv) Fast1/FOXH1, (v) Ku autoantigen, (vi) NMP4, (vii) Mut p53, (viii) SAF-A/hnRNPU, (ix) SATB1, (x) YY1, or (xi) a combination thereof.

28. The method vector of any one of claims 16-27, wherein the S/MAR is upstream of the coding sequence, optionally wherein the S/MAR is no more than 2 kb, 1 kb, 500 bp, or 100 bp upstream of a transcription start site of the coding sequence.

29. The method vector of any one of claims 16-27, wherein the S/MAR is downstream of the coding sequence, optionally wherein the S/MAR is no more than 2 kb, 1 kb, 500 bp, or 100 bp downstream of the coding sequence.

30. The method vector of any one of claims 16-27, wherein the S/MAR is present in an intron of the coding sequence.

31. The method vector of any one of claims 16-30, wherein the expression vector is a circular nucleic acid, optionally wherein the expression vector is a plasmid.

32. The method vector of any one of claims 16-30, wherein the expression vector is a linear nucleic acid.
